(19) United States
(12) Patent Application Publication
(10) Pub. No.: US 2025/0059505 A1
(43) Pub. Date: Feb. 20, 2025

(54) IMPROVED METHODS FOR INDUCING THE MATURATION OF MAMMALIAN CELLS

(71) Applicant: REVERSE BIOENGINEERING, INC., Alameda, CA (US)

(72) Inventor: Michael D. West, Mill Valley, CA (US)

(21) Appl. No.: 18/720,195

(22) PCT Filed: Dec. 14, 2022
(86) PCT No.: PCT/US2022/052803

§ 371 Date: Jun. 14, 2024

Related U.S. Application Data
(60) Provisional application No. 63/289,411, filed on Dec. 14, 2021.

(51) Int. Cl. C12N 5/0735; A61K 31/7088

(52) U.S. Cl. CPC C12N 5/0606; A61K 31/7088; C12N 2501/117; C12N 2501/395; C12N 2501/727; C12N 2506/02

(57) ABSTRACT

Aspects of the present invention include compositions and methods for discovering novel compositions and applying said compositions in treating medical conditions including aging, degenerative disease, and cancer through the modulation of molecular pathways regulating the regenerative and nonregenerative phenotypes of mammalian cells by means of globally or segmentally altering the embryonic-fetal and prenatal/postnatal transitional states of mammalian cells.

### RELATED APPLICATIONS

This application claims priority to U.S. Provisional Application No. 63/289,411, filed Dec. 14, 2021, the entire contents of which are incorporated herein by reference.

### FIELD OF THE INVENTION

The present invention relates to novel compositions and methods for maturing the phenotype of mammalian cells from that corresponding to early embryonic or fetal stages of development to that of later fetal or adult stages of development. The invention is applicable in maturing said cells for use in drug screening as well as in treating medical conditions including aging, degenerative disease, and cancer through the modulation of molecular pathways regulating the regenerative and nonregenerative phenotypes.

### BACKGROUND

Advances in stem cell technology, such as the isolation and propagation in vitro of human pluripotent stem (hPS), including but not limited to human embryonic stem (hES) and human induced pluripotent stem (hiPS) cells, constitute an important new area of medical research. hPS cells have a demonstrated potential to be propagated in the undifferentiated state or alternatively to be induced to differentiate into any and all of the cell types in the human body (Thomson et al., Science 282:1145-1147 (1998)). The unique intrinsic capacity of hPS cells to differentiate into all somatic cell types logically provides a platform for the manufacture of transplantable hPS-derived cells of similar diversity for the treatment for a wide variety of degenerative diseases. While this pluripotency of hES and hiPS cells is currently widely recognized, less recognized, and rarely studied, is the unique capacity of hPS cells cultured in vitro to generate relatively undifferentiated embryonic anlagen.

Even more rarely studied is the potential of hPS cell-derived cells to differentiate into recognized cell types such as cardiomyocytes or osteochondral cells that nevertheless display subtle prenatal, or even prefetal patterns of gene expression that distinguish them from fetal or adult counterparts. Immediately prior to the embryonic-fetal transition (EFT) in vivo, mammalian differentiated cells and tissues such as the skin, heart, and spinal cord show a profound scarless regenerative potential that is progressively lost subsequent to the EFT. In the case of some tissues, such as the human heart, potential for scarless regeneration is detectable for approximately a week past the prenatal-postnatal transition (PPT) period. Given the importance of understanding and modulating tissue regeneration in order to either induce tissue regeneration (iTR) or alternatively, to induce non-cancerous cell or tissue maturation (iTM) or induce cancer cell maturation (iCM) wherein said cancer cells display an embryonic (pre-fetal) pattern of gene expression, there is a need for improved methods of modelling and modulating the maturation of diverse mammalian somatic cells in vitro and in vivo for research, drug screening, and clinical practice.

The potential of pluripotent stem cells and derived embryoid bodies for in vitro self-assembly into 3-dimensional organoids has generated interest as a potential pathway for both obtaining tissue for transplantation (Singh et al, Stem Cells Dev. 2015. 24(23): 2778-95) as well as modeling human embryonic development. The present invention teaches that said organoid formation is a reflection of the intrinsic potential of cells prior to the EFT to undergo tissue generation and/or regeneration. In contrast to embryonic cells, fetal and adult-derived cells often show reduced potential for organogenesis in vitro and epimorphic regeneration in vivo. Epimorphic regeneration, sometimes referred to as “epimorphosis,” refers to a type of tissue regeneration wherein a blastema of relatively undifferentiated mesenchyme proliferates at the site of injury and then the cells differentiate to restore the original tissue histology. The developmental timing of the loss of epimorphic potential cannot be fixed precisely, and likely varies with tissue type, nevertheless, the EFT which occurs at about the end of eight weeks of human development (Carnegie Stage 23; O'Rahilly, R., F. Müller (1987) Developmental Stages in Human Embryos, Including a Revision of Streeter's ‘Horizons’ and a Survey of the Carnegie Collection. Washington, Carnegie Institution of Washington) appears to temporally correspond to the loss of skin regeneration in placental mammals (Walmsley, G. G. et al 2015. Scarless Wound Healing: Chasing the Holy Grail Plast Reconstr Surg. 135(3):907-17). Correlations between species show increased regenerative potential in the embryonic or larval state (reviewed in Morgan, T. H. (1901). Regeneration (New York: The MacMillan Company); also Sanchez Alvarado, A., and Tsonis, P. A. (2006) Bridging the regeneration gap: genetic insights from diverse animal models. Nat. Rev. Genet. 7, 873-884). This suggests that tissue regeneration, as opposed to scarring, reflects the presence of an embryonic as opposed to fetal or adult phenotype, though there is currently no consensus in the scientific community that epimorphic tissue regeneration is a result of an embryonic (pre-natal, more specifically, pre-fetal) pattern of gene expression. In the case of some non-mammalian species, a change in developmental timing (heterochrony) correlates with profound regenerative potential such as is the case in the developmental arrest in larval development (heterochrony) and limb regeneration observed in the Mexican salamander axolotl (A. mexicanum). The profound regenerative potential of A. mexicanum appears to reflect a defect of thyroid hormone signaling which appears to be a signal for metamorphosis (Voss, S. R. et al, Thyroid hormone responsive QTL and the evolution of paedomorphic salamanders. Heredity (2012) 109, 293-298.

Despite these observations, there is currently no understanding of any comparable endocrine signaling pathway in mammalian species that may regulate the maturation of mammalian cells during the EFT and the associated loss of regenerative potential. We previously disclosed compositions and methods related to markers of the EFT in mammalian species and methods of modulating tissue regeneration and cancer diagnosis described in “Compositions and Methods for Induced Tissue Regeneration in Mammalian Species” (International Patent Application Publication No. WO 2014/197421), incorporated herein by reference in its entirety and “Improved Methods for Detecting and Modulating the Embryonic-Fetal Transition in Mammalian Species” (see PCT/US14/40601, filed Jun. 3, 2014 and titled “Compositions and Methods for Induced Tissue Regeneration in Mammalian Species”; and PCT/US2017/036452, filed Jun. 7, 2017 and titled “Improved Methods for Detecting and Modulating the Embryonic-Fetal Transition in Mammalian Species, and see PCT International Application No. PCT/US2020/012640, titled “Compositions and Methods for Detecting Cardiotoxicity,” filed Jan. 7, 2020; U.S. Provisional Application No. 63/274,731, titled “Use of Protocadherins in Methods of Diagnosing and Treating Cancer,” filed Nov. 2, 2021; U.S. Provisional Application No. 63/274,734, titled “Methods and Compositions Used to Modify Chromatin Architecture to Regulate Phenotype in Aging and Cancer,” filed Nov. 2, 2021; and U.S. Provisional Application No. 63/256,286, titled “Methods for Modulating the Regenerative Phenotype in Mammalian Cells,” filed Oct. 15, 2021, contents of each of which are incorporated herein by reference., contents of each of which are incorporated herein by reference). By way of non-limiting example, one such marker for the EFT in diverse mammalian cells is the expression of the gene COX7A1 which begins to be expressed at the EFT and increases in expression during fetal development until adulthood. The aforementioned compositions and methods relating to COX7A1 and other genes regulating the EFT were based in part on the methods allowing the clonal expansion of hPS cell-derived embryonic progenitor (EP) cell lines which provide a means to propagate novel diverse and highly purified cell lineages with a pre-natal pattern of gene expression useful for regenerating tissues such as skin in a scarless manner. Such cell types have important applications in research, and for the manufacture of cell-based therapies (see PCT application Ser. No. PCT/US2006/013519 filed on Apr. 11, 2006 and titled “Novel Uses of Cells With Prenatal Patterns of Gene Expression”; U.S. patent application Ser. No. 11/604,047 filed on Nov. 21, 2006 and titled “Methods to Accelerate the Isolation of Novel Cell Strains from Pluripotent Stem Cells and Cells Obtained Thereby”; and U.S. patent application Ser. No. 12/504,630 filed on Jul. 16, 2009 and titled “Methods to Accelerate the Isolation of Novel Cell Strains from Pluripotent Stem Cells and Cells Obtained Thereby”, each incorporated herein by reference).

In addition, we previously disclosed compositions and methods related to markers of the EFT in mammalian species and their use in non-cancerous somatic cells for inducing cell and tissue maturation (iTM) and induced cancer cell maturation (iCM) of cancer cells that display an embryonic (pre-fetal pattern of gene expression) in “Compositions and Methods for Induced Tissue Regeneration in Mammalian Species” (international patent application publication number WO 2014/197421), incorporated herein by reference in its entirety and “Improved Methods for Detecting and Modulating the Embryonic-Fetal Transition in Mammalian Species” (see PCT/US14/40601, filed Jun. 3, 2014 and titled “Compositions and Methods for Induced Tissue Regeneration in Mammalian Species”; and PCT/US2017/036452, filed Jun. 7, 2017 and titled “Improved Methods for Detecting and Modulating the Embryonic-Fetal Transition in Mammalian Species, and (see PCT International Application No. PCT/US2020/012640, titled “Compositions and Methods for Detecting Cardiotoxicity,” filed Jan. 7, 2020; PCT International Patent Application No. PCT/US2020/025512, titled “Induced tissue regeneration using extracellular vesicles,” filed Mar. 27, 2020; U.S. Provisional Application No. 63/274,731, titled “Use of Protocadherins in Methods of Diagnosing and Treating Cancer,” filed Nov. 2, 2021; U.S. Provisional Application No. 63/274,734, titled “Methods and Compositions Used to Modify Chromatin Architecture to Regulate Phenotype in Aging and Cancer,” filed Nov. 2, 2021; and U.S. Provisional Application No. 63/256,286, titled “Methods for Modulating the Regenerative Phenotype in Mammalian Cells,” filed Oct. 15, 2021, contents of each of which are incorporated herein by reference) contents of each of which are incorporated herein by reference). The aforementioned compositions and methods were based in part on the methods comparing the molecular composition and activities of hPS cell-derived embryonic progenitor (EP) cell lines with adult and cancer cell counterparts.

Nevertheless, additional and improved methods and compositions for maturing mammalian cells from a pre-EFT to a post-EFT pattern of gene expression, or maturing said cells from a fetal to more mature adult pattern of gene expression would be useful in maturing pluripotent stem cell (PSC)-derived cells for use in drug screening or cell transplantation as well as maturing cancer cells that display an embryonic (pre-fetal) pattern of gene expression in diverse types of malignancy.

### SUMMARY

The present invention teaches that molecular mechanisms central to the regulation of regeneration evolved in association with evolution of tetrapods from previous aquatic vertebrates and subsequent evolution of entirely terrestrial vertebrates (the amniotes). Furthermore, the present invention teaches that signaling pathways such as that of thyroid and glucocorticoid hormones which continue to play a role in triggering metamorphosis in extant amphibians such as anurans and axolotls, continues to play an important role in the loss of the regenerative phenotype during EFT and the perinatal transition of amniotes such as mammalian species. Furthermore, the present invention teaches that the in utero development of placental mammals required the regulation of thyroid hormone to mimic that of the previous aquatic mileau of developing anamniotes such as through the expression of deiodinases to protect the developing mammal from maternal thyroid hormone such as T3. Furthermore, the present invention teaches that thyroid hormone and glucocorticoid hormones exert their effects through the ERK1/2 pathway and transcriptional activation of Fos and Jun family members to activate the adult-like nonregenerative phenotype. Furthermore, the present invention teaches that these insights provide novel compositions and methods for advancing the development of embryonic (pre fetal) mammalian cells such as pluripotent stem cell-derived cells in vitro or in vivo to that of an adult phenotype. Said compositions and methods have utility in obtaining fully-adult mammalian cells for use in in vitro drug screening, in maturing cells prior to transplantation, for cancer therapy, and for basic research.

The present disclosure provides novel methods and compositions useful in advancing the developmental phenotype of mammalian from that of an embryonic (pre-fetal) phenotype to that of later fetal or adult cells, e.g., maturing a cell.

In one aspect of the present disclosure, a method is provided for the advancement of development of a mammalian somatic cell from a gene expression phenotype corresponding to an embryonic (pre-fetal) cell to that of a later fetal or adult-like cell wherein said mammalian cell is exposed one or more endocrine factors (e.g., global maturation factors) selected from the group consisting of thyroid hormones T3 or T4, together with one or more of cortisol, dexamethasone, proopiomelanocortin (POMC) and its derivative peptides (e.g., N-terminal peptide of proopiomelanocortin (NPP), alpha melanotropin (αMSH), beta-melanotropin (βMSH), delta-melanocyte-stimulating hormone (δMSH), epsilson-melanocyte-stimulating hormone (εMSH), corticotropin, beta-lipotropin, gamma lipotropin, beta-endorphin, and met-enkephalin), a MAP kinase activator such as an ERK1/2 activator, said ERK1/2 activator by way of nonlimiting example being baicalein or baicalin, lamin A, FGF7, IGF2, and Growth hormone (GH). According to one embodiment, the endocrine factor (e.g., global maturation factors) is thyroid hormones T3 or T4 and one or more additional endocrine factor. According to one embodiment, the one or more additional endocrine factor (e.g., global maturation factors) is selected from the group consisting of cortisol, dexamethasone, proopiomelanocortin (POMC) and its derivative peptides (e.g., N-terminal peptide of proopiomelanocortin (NPP), alpha melanotropin (αMSH), beta-melanotropin (βMSH), delta-melanocyte-stimulating hormone (δMSH), epsilson-melanocyte-stimulating hormone (εMSH), corticotropin, beta-lipotropin, gamma lipotropin, beta-endorphin, and met-enkephalin), a MAP kinase activator such as an ERK1/2 activator, said ERK1/2 activator by way of nonlimiting example being baicalein or baicalin, lamin A, FGF7, IGF2, and Growth hormone (GH). According to one embodiment, the one or more additional endocrine factor (e.g., global maturation factors) is cortisol. According to one embodiment, the one or more additional endocrine factor (e.g., global maturation factors) is dexamethasone. According to one embodiment, the one or more additional endocrine factor (e.g., global maturation factors) is proopiomelanocortin (POMC) and/or one or more of POMC derivative peptides (e.g., N-terminal peptide of proopiomelanocortin (NPP), alpha melanotropin (αMSH), beta-melanotropin (βMSH), delta-melanocyte-stimulating hormone (δMSH), epsilson-melanocyte-stimulating hormone (εMSH), corticotropin, beta-lipotropin, gamma lipotropin, beta-endorphin, and met-enkephalin). According to one embodiment, the one or more additional endocrine factor (e.g., global maturation factors) is a MAP kinase activator such as an ERK1/2 activator, said ERK1/2 activator by way of nonlimiting example being baicalein or baicalin. According to one embodiment, the one or more additional endocrine factor (e.g., global maturation factors) is lamin A. According to one embodiment, the one or more additional endocrine factor (e.g., global maturation factors) is FGF7. According to one embodiment, the one or more additional endocrine factor (e.g., global maturation factors) is IGF2. According to one embodiment, the one or more additional endocrine factor (e.g., global maturation factors) is Growth hormone (GH).

In another embodiment compositions and methods are provided for the maturation of mammalian (including human) somatic cell types using endocrine signaling pathways.

In another embodiment compositions and methods are provided for the maturation of mammalian (including human) somatic cell types using the endocrine factors, thyroid hormones T3 and T4.

In another embodiment compositions and methods are provided for the maturation of mammalian (including human) somatic cell types using the endocrine factor, T3.

In another embodiment compositions and methods are provided for the maturation of mammalian (including human) somatic cell types using multiple endocrine factors comprising one or more of: T3, T4, cortisol, dexamethasone, FGF7, IGF2, and Growth hormone (GH).

In another embodiment compositions and methods are provided for the maturation of mammalian cancer cells expressing an embryonic (pre-fetal) pattern of gene expression and maturing them to a fetal or adult pattern of gene expression with increased COX7A1 expression and increased mesenchymal gene expression as evidenced by the increased expression of COL1A1.

In another embodiment compositions and methods are provided for the maturation of mammalian (including human) somatic cell types by exogenous administration of COX7A1 nucleic acids to down-regulate MANEAL gene expression.

In another embodiment compositions and methods are provided for the maturation of mammalian (including human) cancer cell types as evidenced by the down-regulation of TERT by means of the exogenous administration of COX7A1.

In another aspect of the present disclosure, a method is provided for the advancement of development of a mammalian somatic cell from a gene expression phenotype corresponding to an embryonic (pre-fetal) cell to that of a later fetal or adult-like cell wherein said mammalian cell is exposed to thyroid hormone.

In another aspect of the present disclosure, a method is provided for the advancement of development of a mammalian somatic cell from a gene expression phenotype corresponding to an embryonic (pre-fetal) cell to that of a later fetal or adult-like cell wherein said mammalian cell is exposed to the thyroid hormone T4.

In another aspect of the present disclosure, a method is provided for the advancement of development of a mammalian somatic cell from a gene expression phenotype corresponding to an embryonic (pre-fetal) cell to that of a later fetal or adult-like cell wherein said mammalian cell is exposed to the thyroid hormone T3 together with a glucocorticoid hormone such as cortisol or dexamethasone.

In another aspect of the present disclosure, a method is provided for the advancement of development of a mammalian somatic cell from a gene expression phenotype corresponding to an embryonic (pre-fetal) cell to that of a later fetal or adult-like cell wherein said mammalian cell is exposed to the thyroid hormone T4 together with a glucocorticoid hormone such as cortisol or dexamethasone.

In another aspect of the present disclosure, a method is provided for the advancement of development of a mammalian somatic cell from a gene expression phenotype corresponding to an embryonic (pre-fetal) cell to that of a later fetal or adult-like cell wherein said mammalian cell is exposed to the thyroid hormone T3 together with a glucocorticoid hormone precursor such as proopiomelanocortin (POMC) and POMC derivative peptides (e.g., N-terminal peptide of proopiomelanocortin (NPP), alpha melanotropin (αMSH), beta-melanotropin (βMSH), delta-melanocyte-stimulating hormone (δMSH), epsilson-melanocyte-stimulating hormone (εMSH), corticotropin, beta-lipotropin, gamma lipotropin, beta-endorphin, and met-enkephalin).

In another aspect of the present disclosure, a method is provided for the advancement of development of a mammalian somatic cell from a gene expression phenotype corresponding to an embryonic (pre-fetal) cell to that of a later fetal or adult-like cell wherein said mammalian cell is exposed to the thyroid hormone T4 together with a glucocorticoid hormone precursor such as proopiomelanocortin (POMC) and POMC derivative peptides (e.g., N-terminal peptide of proopiomelanocortin (NPP), alpha melanotropin (αMSH), beta-melanotropin (βMSH), delta-melanocyte-stimulating hormone (δMSH), epsilson-melanocyte-stimulating hormone (εMSH), corticotropin, beta-lipotropin, gamma lipotropin, beta-endorphin, and met-enkephalin).

In another aspect of the present disclosure, a method is provided for the advancement of development of a mammalian somatic cell from a gene expression phenotype corresponding to an embryonic (pre-fetal) cell to that of a later fetal or adult-like cell wherein said mammalian cell is exposed to the thyroid hormone T3 together with a an ERK1/2 activator.

In another aspect of the present disclosure, a method is provided for the advancement of development of a mammalian somatic cell from a gene expression phenotype corresponding to an embryonic (pre-fetal) cell to that of a later fetal or adult-like cell wherein said mammalian cell is exposed to the thyroid hormone T4 together with a an ERK1/2 activator.

In one aspect of the present disclosure, a method is provided for the advancement of development of a mammalian cancer cell from a gene expression phenotype corresponding to an embryonic (pre-fetal) cell to that of a later fetal or adult-like cell wherein said mammalian cell is exposed one or more of the thyroid hormones T3 or T4, together with one or more of cortisol, dexamethasone, proopiomelanocortin (POMC) and its derivative peptides (e.g., N-terminal peptide of proopiomelanocortin (NPP), alpha melanotropin (αMSH), beta-melanotropin (βMSH), delta-melanocyte-stimulating hormone (δMSH), epsilson-melanocyte-stimulating hormone (εMSH), corticotropin, beta-lipotropin, gamma lipotropin, beta-endorphin, and met-enkephalin), a MAP kinase activator such as an ERK1/2 activator, said ERK1/2 activator by way of nonlimiting example being baicalein or baicalin, and lamin A.

In another aspect of the present disclosure, compositions and methods are provided for the expression of the genes for iTR inhibitory factors including combinations of ACAT2, C18orf56, CAT, COMT, COX7A1, DYNLT3, ELOVL6, FDPS, IAH1, INSIG1, LOC205251, MAOA, RPS7, SHMT1, TRIM4, TSPYL5, or ZNF280D previously disclosed in PCT/US14/40601, filed Jun. 3, 2014 and titled “Compositions and Methods for Induced Tissue Regeneration in Mammalian Species,” or ADIRF, C10orf11, CAT, CCDC144B, COMT, COX7A1, KRBOX1, LINC00654, LINC00839, LINC01116, MEG3, MIR4458HG, PCDHGA12, PCDHGB3, PCDHGB5, PLPP7 (PPAPDC3), POMC, PRR34, PRSS3, PTCHD3, PTCHD3P1, SPESP1, TRIM4, USP32P1, ZNF300P1, or ZNF572 previously disclosed in PCT/US2017/036452, filed Jun. 7, 2017 and titled “Improved Methods for Detecting and Modulating the Embryonic-Fetal Transition in Mammalian Species,” or ALS2CR11, C2CD6, ANKRD7, ANKRD65, BACE2, BHMT2, C22orf26, CADPS2, CALHM2, CCDC36, CCDC89, CCDC125, CCDC144B, CLDN11, CTSF, DDX43, DNAJC15, EGFLAM, ESPNL, FAM24B, FGF7, FKBP9L, FLG-AS1, FRG1B, GPAT2, GYPE, HENMT1, HIST2H2BA, IRAK4, LINC00865, LOC283788, LOC100233156, LRRK2, MAP10, MEG8, MEG9, MIRLET7HG, NKAPL, PAX8-AS1, PRPH2, PRR34-AS1, RP5-1043L13.1, RP11-134021.1, SVIL-AS1, TEKT4P2, ZNF578, ZNF585B, ZNF736, or ZNF790-AS1previously disclosed U.S. Provisional Application No. 63/256,286, titled “Methods for Modulating the Regenerative Phenotype in Mammalian Cells,” filed Oct. 15, 2021 (contents of each of which are incorporated herein by reference).

In one aspect of the present disclosure, a method is provided for the advancement of development of a mammalian somatic cell from a gene expression phenotype corresponding to an embryonic (pre-fetal) cell to that of a later fetal or adult-like cell wherein said mammalian cell is exposed one or more of the thyroid hormones T3 or T4, together with one or more of cortisol, dexamethasone, proopiomelanocortin (POMC) and its derivative peptides (e.g., N-terminal peptide of proopiomelanocortin (NPP), alpha melanotropin (αMSH), beta-melanotropin (βMSH), delta-melanocyte-stimulating hormone (δMSH), epsilson-melanocyte-stimulating hormone (εMSH), corticotropin, beta-lipotropin, gamma lipotropin, beta-endorphin, and met-enkephalin), a MAP kinase activator such as an ERK1/2 activator, said ERK1/2 activator by way of nonlimiting example being baicalein or baicalin, and lamin A.

In one embodiment, the adult mammalian somatic cells are human.

In one embodiment, the one or more iTR factors are administered by viral vector. In one embodiment, the viral vector is an adeno-associated virus.

In one embodiment, the one or more iTR factors are one or more nucleic acids encoding PURPL.

In another aspect, the present disclosure provides for a method of reprogramming adult mammalian somatic cells to a regenerative phenotype, the method comprising contacting the adult mammalian somatic cells with one or more nucleic acids encoding RNAi constructs targeting one or more of induced tissue regeneration (iTR) inhibitory genes: ALS2CR11, C2CD6, ANKRD7, ANKRD65, BACE2, BHMT2, C22orf26, CADPS2, CALHM2, CCDC36, CCDC89, CCDC125, CCDC144B, CLDN11, CTSF, DDX43, DNAJC15, EGFLAM, ESPNL, FAM24B, FGF7, FKBP9L, FLG-AS1, FRG1B, GPAT2, GYPE, HENMT1, HIST2H2BA, IRAK4, LINC00865, LOC283788, LOC100233156, LRRK2, MAP10, MEG8, MEG9, MIRLET7HG, NKAPL, PAX8-AS1, PRPH2, PRR34-AS1, RP5-1043L13.1, RP11-134021.1, SVIL-AS1, TEKT4P2, ZNF578, ZNF585B, ZNF736, or ZNF790-AS1, or a combination thereof, thereby inducing a regenerative phenotype in the adult mammalian somatic cells or the corresponding tissues.

In one embodiment, the mammalian somatic cells are human.

In one embodiment, the one or more iTM or iCM factors are administered in vitro.

In one embodiment, the one or more iTM or iCM factors are administered in vivo.

In one embodiment, the one or more iTM or iCM factors are administered using a gene therapy vector.

In one embodiment, the one or more iTM or iCM factors are administered using a viral gene therapy vector.
viral vector. In one embodiment, the viral vector is an adeno-associated virus.

In one embodiment, the assay for determining the advancement of cells toward an adult-like pattern of gene expression is determined by measuring the expression of the gene COX7A1 before and after treatment with the iTM or iCM factor.

In another aspect, the present disclosure provides for a method of advancing the development of mammalian somatic cells from that of an embryonic (pre-fetal pattern of gene expression) to that of a later fetal or adult-like pattern of gene expression comprising: (a) obtaining embryonic (pre-fetal) cells by differentiating pluripotent stem cells such that embryonic (pre-fetal) differentiated cells are obtained; (b) contacting the cells with one or more of induced cell and tissue maturation (iTM) factors described herein; (c) assaying the extent of maturation of said embryonic cells utilizing markers expressed in adult, but not embryonic cells by way of nonlimiting example, the level of expression of mRNA from the gene COX7A1 or its corresponding protein product.

In one embodiment, the one or more nucleic acids encoding one or more iTM or iCM factors are administered in combination with hydrogel.

In one aspect, the present disclosure provides a method of maturing a mammalian cell that expresses an embryonic (pre-fetal) pattern of gene expression into a cell that expresses markers of fetal or adult cells, said method comprising administering one or more endocrine factors to said cells.

In some embodiments, the endocrine factors are selected from the group consisting of T3, T4, cortisol, dexamethasone, proopiomelanocortin (POMC) and POMC derivative peptides, a MAP kinase activator, FGF7, IGF2, and Growth hormone (GH), wherein the POMC derivatives are N-terminal peptide of proopiomelanocortin (NPP), alpha melanotropin ((αMSH), beta-melanotropin (BMSH), delta-melanocyte-stimulating hormone (δMSH), epsilson-melanocyte-stimulating hormone (εMSH), corticotropin, beta-lipotropin, gamma lipotropin, beta-endorphin, and met-enkephalin, wherein the MAP kinase is an ERK1/2 activator, e.g., baicalein or baicalin, and lamin A.

In some embodiments, the endocrine factors are 1) T3 or T4; and 2) one or more of cortisol, dexamethasone, proopiomelanocortin (POMC) and POMC derivative peptides, a MAP kinase activator, FGF7, IGF2, and Growth hormone (GH), wherein the POMC derivatives are N-terminal peptide of proopiomelanocortin (NPP), alpha melanotropin (αMSH), beta-melanotropin (βMSH), delta-melanocyte-stimulating hormone (δMSH), epsilson-melanocyte-stimulating hormone (εMSH), corticotropin, beta-lipotropin, gamma lipotropin, beta-endorphin, and met-enkephalin, wherein the MAP kinase is an ERK1/2 activator, e.g., baicalein or baicalin, and lamin A.

In some embodiments, the endocrine factors are 1) T3 or T4; and 2) one or more of cortisol, dexamethasone, FGF7, IGF2, and Growth hormone (GH). In some embodiments, the endocrine factors are 1) T3 or T4; and 2) ERK1/2 activator. In some embodiments, wherein the endocrine factors are 1) T3 or T4; and 2) FGF7. In some embodiments, the endocrine factors are 1) T3 or T4; and 2) one or more of proopiomelanocortin (POMC) and POMC derivative peptides, wherein the POMC derivatives are N-terminal peptide of proopiomelanocortin (NPP), alpha melanotropin (αMSH), beta-melanotropin (βMSH), delta-melanocyte-stimulating hormone (δMSH), epsilson-melanocyte-stimulating hormone (εMSH), corticotropin, beta-lipotropin, gamma lipotropin, beta-endorphin, and met-enkephalin.

In some embodiments, the method, further comprising administering one or more induced tissue maturation (iTM) factors.

In some embodiments, the one or more iTM factors are one or more tissue regeneration (TR) inhibitory genes, one or more inhibitors of one or more tissue regeneration (TR) activator genes, or a combination thereof.

In some embodiments, the one or more TR inhibitory genes are selected from the group consisting of ACAT2, C18orf56, CAT, COMT, COX7A1, DYNLT3, ELOVL6, FDPS, IAH1, INSIG1, LOC205251, MAOA, RPS7, SHMT1, TRIM4, TSPYL5, or ZNF280D.

In some embodiments, the one or more TR inhibitory genes are selected from the group consisting of ADIRF, C10orf11, CAT, CCDC144B, COMT, COX7A1, KRBOX1, LINC00654, LINC00839, LINC01116, MEG3, MIR4458HG, PCDHGA12, PCDHGB3, PCDHGB5, PLPP7 (PPAPDC3), POMC, PRR34, PRSS3, PTCHD3, PTCHD3P1, SPESP1, TRIM4, USP32P1, ZNF300P1, or ZNF572.

In some embodiments, the one or more TR inhibitory genes are selected from the group consisting of ALS2CR11, C2CD6, ANKRD7, ANKRD65, BACE2, BHMT2, C22orf26, CADPS2, CALHM2, CCDC36, CCDC89, CCDC125, CCDC144B, CLDN11, CTSF, DDX43, DNAJC15, EGFLAM, ESPNL, FAM24B, FGF7, FKBP9L, FLG-AS1, FRG1B, GPAT2, GYPE, HENMT1, HIST2H2BA, IRAK4, LINC00865, LOC283788, LOC100233156, LRRK2, MAP10, MEG8, MEG9, MIRLET7HG, NKAPL, PAX8-AS1, PRPH2, PRR34-AS1, RP5-1043L13.1, RP11-134021.1, SVIL-AS1, TEKT4P2, ZNF578, ZNF585B, ZNF736, or ZNF790-AS1. In some embodiments, the TR inhibitory gene is FGF7.

In some embodiments, the one of more TR activator genes are selected from the group consisting of AFF3, CBCAQH03 5, DLX1, DRD11P, F2RL2, FOXD1, LOC728755, LOC791120, MN1, OXTR, PCDHB2, PCDHB17, RAB3IP, SIX1, and WSB1.

In some embodiments, the one of more TR activator genes are selected from the group consisting of ADGRV1, AFF3, ALDH5A1, ALX1, AMH, B4GALNT4, C14orf39, CHKB-CPT1B, CPT1B, DOC2GP, DPY19L2, DSG2, FAM157A, FAM157B, FOXD4L4, FSIP2, GDF1, GRIN3B, H2BFXP, L3MBTL1, LIN28B, LINC00649, LINC01021, LINC01116, NAALAD2, PAQR6, members of the alpha clustered protocadherin locus A2-11, members of the beta clustered protocadherin locus B2-17, PCDHGB4, PCDHGB6, PLPPR3, PRR5L, RGPD1, SLCO1A2, TSPAN11, TUBB2B, ZCCHC18, ZNF497, and ZNF853.

In some embodiments, the one of more TR activator genes are selected from the group consisting of AC108142.1, AGA, AQP7P1, AQP7P3, BAHD1, BBOX1, C11orf35 (LMNTD2), CASC9, CBX2, CCDC144NL, CHRM3, CPAMD8, FAR2P1, FAR2P2, FAR2P3, FIRRE, IGF2BP1, LINC00649, LINC02315, LOC644919, MED15P9, PCAT7, PKP3, POTEE, POTEF, PURPL, RGPD2, WDR72, WRN, and LMNB1.

In some embodiments, the TR activator gene is PURPL.

In some embodiments, the one or more inhibitors of one or more tissue regeneration (TR) activator genes is inhibitory RNA (RNAi).

In some embodiments, the one or more iTM factors are administered by viral vector. In some embodiments, the viral vector is an adeno-associated virus.

In some embodiments, the cells are human. In some embodiments, the cells are canine. In some embodiments, the cells are feline.

In some embodiments, wherein the one or more endocrine factors and/or one or more iTM factors are administered to the cell for at least 1 day, at least 2 days, at least 3 days, at least 4 days, at least 5 days, at least 6 days, at least 7 days, at least 8 days, at least 9 days, at least 10 days, at least 11 days, at least 12 days, at least 12 days, at least 13 days, at least 14 days, at least 15 days, at least 16 days, at least 17 days, at least 18 days, at least 19 days, or at least 20 days. In some embodiments, wherein the one or more endocrine factors and/or one or more iTM factors are administered to the cell for up to 1 day, up to 2 days, up to 3 days, up to 4 days, up to 5 days, up to 6 days, up to 7 days, up to 8 days, up to 9 days, up to 10 days, up to 11 days, up to 12 days, up to 12 days, up to 13 days, up to 14 days, up to 15 days, up to 16 days, up to 17 days, up to 18 days, up to 19 days, or up to 20 days.

In some embodiments, the cell is a pluripotent stem cell.

In some embodiments, the pluripotent stem cell is derived from a somatic cell.

In some embodiments, the somatic cell is selected from the group consisting of cardiac cells, stomach cells, neural cells, lung cells, cells of the ear, cells of the olfactory system, reproductive cells, pancreatic cells, gastrointestinal cells, thyroid cells, epithelial cells, bladder cells, blood cells, respiratory tract cells, salivary gland cells, adipocytes, cells of the eye, liver cells, muscle cells, kidney cells, and immune system cells.

In some embodiments, the one or more iTM factors are administered in vitro. In some embodiments, the one of more iTM factors are administered in vivo. In some embodiments, the one or more iTM factors are administered by viral vector. In some embodiments, the viral vector is an adeno-associated virus.

In some embodiments, the cell has reduced transcription of the TERT gene compared to the level of transcription of the TERT gene in the cell prior to administration of the one or more endocrine factors and/or the one or more iTM factors.

In some embodiments, the cell has reduced transcription of the MANEAL gene compared the level of transcription of the MANEAL gene in the cell prior to administration of the one or more endocrine factors and/or the one or more iTM factors.

In some embodiments, the cells has increased COX7A1 expression compared the level of transcription of the COX7A1 gene in the cell prior to administration of the one or more endocrine factors and/or the one or more iTM factors.

In some embodiments, the cells has increased COL1A1 expression compared the level of transcription of the COL1A1 gene in the cancer cell prior to administration of the one or more endocrine factors and/or the one or more iTM factors.

In another aspect, the present disclosure provides a method of maturing a cancer cell with an embryonic pattern of gene expression and a glycolytic phenotype, said method comprising administering one or more endocrine factors to said cancer cell.

In some embodiments, the endocrine factors are selected from the group consisting of T3, T4, cortisol, dexamethasone, IGF2, growth hormone, proopiomelanocortin (POMC) and POMC derivative peptides, wherein POMC derivative peptides are N-terminal peptide of proopiomelanocortin (NPP), alpha melanotropin ((αMSH), beta-melanotropin (βMSH), delta-melanocyte-stimulating hormone (δMSH), epsilson-melanocyte-stimulating hormone (εMSH), corticotropin, beta-lipotropin, gamma lipotropin, beta-endorphin, and met-enkephalin.

In some embodiments, the endocrine factors are 1) T3 or T4; and 2) one or more of cortisol, dexamethasone, proopiomelanocortin (POMC) and POMC derivative peptides, a MAP kinase activator, FGF7, IGF2, and Growth hormone (GH), wherein the POMC derivatives are N-terminal peptide of proopiomelanocortin (NPP), alpha melanotropin (αMSH), beta-melanotropin (βMSH), delta-melanocyte-stimulating hormone (δMSH), epsilson-melanocyte-stimulating hormone (εMSH), corticotropin, beta-lipotropin, gamma lipotropin, beta-endorphin, and met-enkephalin, wherein the MAP kinase is an ERK1/2 activator, e.g., baicalein or baicalin, and lamin A.

In some embodiments, the endocrine factors are 1) T3 or T4; and 2) one or more of cortisol, dexamethasone, FGF7, IGF2, and Growth hormone (GH). In some embodiments, the endocrine factors are 1) T3 or T4; and 2) ERK1/2 activator. In some embodiments, wherein the endocrine factors are 1) T3 or T4; and 2) FGF7. In some embodiments, the endocrine factors are 1) T3 or T4; and 2) one or more of proopiomelanocortin (POMC) and POMC derivative peptides, wherein the POMC derivatives are N-terminal peptide of proopiomelanocortin (NPP), alpha melanotropin (αMSH), beta-melanotropin (βMSH), delta-melanocyte-stimulating hormone (δMSH), epsilson-melanocyte-stimulating hormone (εMSH), corticotropin, beta-lipotropin, gamma lipotropin, beta-endorphin, and met-enkephalin.

In some embodiments, the method further comprising administering one or more induced tissue maturation (iTM) factors.

In some embodiments, the one or more iTM factors are one or more tissue regeneration (TR) inhibitory genes, one or more inhibitors of one or more tissue regeneration (TR) activator genes, or a combination thereof.

In some embodiments, the one or more TR inhibitory genes are selected from the group consisting of ACAT2, C18orf56, CAT, COMT, COX7A1, DYNLT3, ELOVL6, FDPS, IAH1, INSIG1, LOC205251, MAOA, RPS7, SHMT1, TRIM4, TSPYL5, or ZNF280D.

In some embodiments, the one or more TR inhibitory genes are selected from the group consisting of ADIRF, C10orf11, CAT, CCDC144B, COMT, COX7A1, KRBOX1, LINC00654, LINC00839, LINC01116, MEG3, MIR4458HG, PCDHGA12, PCDHGB3, PCDHGB5, PLPP7 (PPAPDC3), POMC, PRR34, PRSS3, PTCHD3, PTCHD3P1, SPESP1, TRIM4, USP32P1, ZNF300P1, or ZNF572.

In some embodiments, the one or more TR inhibitory genes are selected from the group consisting of ALS2CR11, C2CD6, ANKRD7, ANKRD65, BACE2, BHMT2, C22orf26, CADPS2, CALHM2, CCDC36, CCDC89, CCDC125, CCDC144B, CLDN11, CTSF, DDX43, DNAJC15, EGFLAM, ESPNL, FAM24B, FGF7, FKBP9L, FLG-AS1, FRG1B, GPAT2, GYPE, HENMT1, HIST2H2BA, IRAK4, LINC00865, LOC283788, LOC100233156, LRRK2, MAP10, MEG8, MEG9, MIRLET7HG, NKAPL, PAX8-AS1, PRPH2, PRR34-AS1, RP5-1043L13.1, RP11-134021.1, SVIL-AS1, TEKT4P2, ZNF578, ZNF585B, ZNF736, or ZNF790-AS1. In some embodiments, the TR inhibitory gene is FGF7.

In some embodiments, the one of more TR activator genes are selected from the group consisting of AFF3, CBCAQH03 5, DLX1, DRD11P, F2RL2, FOXD1, LOC728755, LOC791120, MN1, OXTR, PCDHB2, PCDHB17, RAB3IP, SIX1, and WSB1.

In some embodiments, the one of more TR activator genes are selected from the group consisting of ADGRV1, AFF3, ALDH5A1, ALX, AMH, B4GALNT4, C14orf39, CHKB-CPT1B, CPT1B, DOC2GP, DPY19L2, DSG2, FAM157A, FAM157B, FOXD4L4, FSIP2, GDF1, GRIN3B, H2BFXP, L3MBTL1, LIN28B, LINC00649, LINC01021, LINC01116, NAALAD2, PAQR6, members of the alpha clustered protocadherin locus A2-11, members of the beta clustered protocadherin locus B2-17, PCDHGB4, PCDHGB6, PLPPR3, PRR5L, RGPD1, SLCO1A2, TSPAN11, TUBB2B, ZCCHC18, ZNF497, and ZNF853.

In some embodiments, the one of more TR activator genes are selected from the group consisting of AC108142.1, AGA, AQP7P1, AQP7P3, BAHD1, BBOX1, C11orf35 (LMNTD2), CASC9, CBX2, CCDC144NL, CHRM3, CPAMD8, FAR2P1, FAR2P2, FAR2P3, FIRRE, IGF2BP1, LINC00649, LINC02315, LOC644919, MED15P9, PCAT7, PKP3, POTEE, POTEF, PURPL, RGPD2, WDR72, WRN, and LMNB1.

In some embodiments, the TR activator gene is PURPL.

In some embodiments, the one or more inhibitors of one or more tissue regeneration (TR) activator genes is inhibitory RNA (RNAi). In some embodiments, the one or more iTM factors are administered by viral vector. In some embodiments, the viral vector is an adeno-associated virus.

In some embodiments, the cancer cell is human. In some embodiments, the cancer cells are canine. In some embodiments, the cells are feline.

In some embodiments, wherein the one or more endocrine factors and/or one or more iTM factors are administered to the cell for at least 1 day, at least 2 days, at least 3 days, at least 4 days, at least 5 days, at least 6 days, at least 7 days, at least 8 days, at least 9 days, at least 10 days, at least 11 days, at least 12 days, at least 12 days, at least 13 days, at least 14 days, at least 15 days, at least 16 days, at least 17 days, at least 18 days, at least 19 days, or at least 20 days. In some embodiments, wherein the one or more endocrine factors and/or one or more iTM factors are administered to the cell for up to 1 day, up to 2 days, up to 3 days, up to 4 days, up to 5 days, up to 6 days, up to 7 days, up to 8 days, up to 9 days, up to 10 days, up to 11 days, up to 12 days, up to 12 days, up to 13 days, up to 14 days, up to 15 days, up to 16 days, up to 17 days, up to 18 days, up to 19 days, or up to 20 days.

In some embodiments, wherein the one or more endocrine factors are administered to the cell for at least 1 day, at least 2 days, at least 3 days, at least 4 days, at least 5 days, at least 6 days, at least 7 days, at least 8 days, at least 9 days, at least 10 days, at least 11 days, at least 12 days, at least 12 days, at least 13 days, at least 14 days, at least 15 days, at least 16 days, at least 17 days, at least 18 days, at least 19 days, or at least 20 days. In some embodiments, wherein the one or more iTM factors are administered to the cell for at least 1 day, at least 2 days, at least 3 days, at least 4 days, at least 5 days, at least 6 days, at least 7 days, at least 8 days, at least 9 days, at least 10 days, at least 11 days, at least 12 days, at least 12 days, at least 13 days, at least 14 days, at least 15 days, at least 16 days, at least 17 days, at least 18 days, at least 19 days, or at least 20 days.

In some embodiments, wherein the one or more endocrine factors are administered to the cell for up to 1 day, up to 2 days, up to 3 days, up to 4 days, up to 5 days, up to 6 days, up to 7 days, up to 8 days, up to 9 days, up to 10 days, up to 11 days, up to 12 days, up to 12 days, up to 13 days, up to 14 days, up to 15 days, up to 16 days, up to 17 days, up to 18 days, up to 19 days, or up to 20 days. In some embodiments, wherein the one or more iTM factors are administered to the cell for up to 1 day, up to 2 days, up to 3 days, up to 4 days, up to 5 days, up to 6 days, up to 7 days, up to 8 days, up to 9 days, up to 10 days, up to 11 days, up to 12 days, up to 12 days, up to 13 days, up to 14 days, up to 15 days, up to 16 days, up to 17 days, up to 18 days, up to 19 days, or up to 20 days.

In some embodiments, the cancer cell is a carcinoma cell. In some embodiments, the cancer cell is an adenocarcinoma cell. In some embodiments, the cancer cell is a sarcoma cell.

In some embodiments, the cancer cell has reduced transcription of the TERT gene compared to the level of transcription of the TERT gene in the cancer cell prior to administration of the one or more endocrine factors and/or the one or more iTM factors.

In some embodiments, the cells has reduced transcription of the MANEAL gene compared the level of transcription of the MANEAL gene in the cancer cell prior to administration of the one or more endocrine factors and/or the one or more iTM factors.

In some embodiments, the cells has increased COX7A1 expression compared the level of transcription of the COX7A1 gene in the cell prior to administration of the one or more endocrine factors and/or the one or more iTM factors.

In some embodiments, the cells has increased COL1A1 expression compared the level of transcription of the COL1A1 gene in the cancer cell prior to administration of the one or more endocrine factors and/or the one or more iTM factors.

### BRIEF DESCRIPTION OF THE DRAWINGS

FIG. 1. Expression of COX7A1 in Pre-EFT vs Post-EFT cells and in cancer cell lines. FPKM reads from the gene COX7A1 which is expressed post-EFT in diverse mammalian cell types and cancer cells in the CSC state. are shown for cultured pluripotent stem cells (four hES cell lines and one iPS cell line); 40 diverse clonal embryonic progenitor cell types including progenitors that are fully-differentiated while retaining a pre-EFT phenotype; 91 diverse cultured human cell types from all three germ layers; 18 diverse cultured epithelial cell types; 39 diverse sarcoma cell lines from diverse tissues; 35 diverse carcinoma and adenocarcinoma cell lines originating from diverse tissues; and four blood cell cancer lines.

FIGS. 2A-2D. Differentiation vs Maturation and the Immature Nature of PSC-Derived Cells. FIG. 2A) The differentiation marker COL2A1 characteristic of cartilage is expressed only in chondrogenic progenitors (“Chondrogenic”) when differentiated for two weeks in the presence of TGFbeta family members (“D”) compared to control initial conditions (“C”). “PSC” designates PSC-derived clonal embryonic progenitor cell line 4D20.8 and “Adult” designates adult-derived bone marrow MSCs. FIG. 2B) The differentiation marker UCP1 characteristic of brown adipocytes is expressed only in brown adipocyte progenitors (“Brown Adipocytes”) when differentiated for two weeks in the presence of adipogenic conditions (“D”) compared to control initial conditions (“C”). “PSC” designates PSC-derived clonal embryonic progenitor cell line NP88 and “Adult” designates fetal-derived brown preadipocytes. FIG. 2C) The differentiation marker NNIT3 (cadiac troponin), characteristic of cardiomyocytes is expressed only in cardiac progenitors (“Cardiac”) when differentiated into beating cardiac cells (“D”) compared to control initial conditions (“C”). “PSC” designates PSC-derived cardiac embryonic progenitors, Fetal refers to fetal ventricular heart muscle aged 9-16 weeks of gestation, and “Adult” designates adult-derived ventricular heart muscle. FIG. 2D) The EFT maturation marker COX7A1 is not expressed in either chondrocyte, brown adipocyte, or cardiac cells that are differentiated to a similar degree to adult counterparts, but are expressed in MSCs, fetal brown preadipocytes, and fetal ventricular heart muscle regardless of the differentiated state of the cells demonstrating the independence of differentiation and maturation as the terms are used herein.

FIG. 3. Volcano Plots of transcription factor binding to chromatin as assayed using TOBIAS for embryonic and adult chodrogenic mesenchyme (cell lines 4D20.8 and MSCs respectively) and embryonic and adult vascular endothelium (cell lines 30MV2-6 and human aortic endothelium (HAEC) respectively).

FIG. 4. Expression of the proopiomelanocortin (POMC) gene Pre-EFT vs Post-EFT cells and in cancer cell lines. FPKM reads from the gene POMC which is expressed post-EFT in diverse mammalian cell types and cancer cells in the CSC state. are shown for cultured pluripotent stem cells (four hES cell lines and one iPS cell line); 40 diverse clonal embryonic progenitor cell types including progenitors that are fully-differentiated while retaining a pre-EFT phenotype; 91 diverse cultured human cell types from all three germ layers; 18 diverse cultured epithelial cell types; 39 diverse sarcoma cell lines from diverse tissues; 35 diverse carcinoma and adenocarcinoma cell lines originating from diverse tissues; and four blood cell cancer lines.

FIGS. 5A-5C. COX7A1-Based Segmental iTM generated in PSC-derived embryonic progenitors and segmental iCM in the cancer cell line HT1080 with an embryonic phenotype. FIG. 5A) Down-regulation of the embryonic marker PCDHB2 in embryonic progenitors and cancer cells. FIG. 5B). Lack of up-regulation of the adult marker PCDHGA12 in embryonic progenitors and cancer cells. FIG. 5C). Down-regulation of the embryonic marker TERT in cancer cells.

FIGS. 6A-6B. Tumor Suppressive Effect of the Segmental iCM gene COX7A1 in Cancer Cells with an Embryonic Pattern of Gene Expression. The tumor cell line HT1080 was infected with lentivirus expression COX7A1 or eGFP control sequence and the resulting cells were passaged in vitro (FIG. 6A) or in vivo in NOD/SCID mice (FIG. 6B) to measure the relative rates of cell proliferation and tumor growth respectively.

FIGS. 7A-7B. Metabolic Alterations in PSC-derived embryonic progenitors and HT1080 cancer cells following the administration of lentivirus expressing COX7A1 or eGFP controls. FIG. 7A) Levels of the glycolytic intermediate glucose 6-phosphate. FIG. 7B) Levels of the long-chain fatty acid conjugate palmitoylcarnitine.

FIGS. 8A-8C. Expression of the Gene MANEAL in Embryonic, Adult, and Cancer Cells and in Cells Treated with the Segmental iCM factor COX7A1. FIG. 8A) FPKM reads from the gene MANEAL which is expressed at reduced levels post-EFT (with the exception of CNS cells and hepatocytes) and markedly up-regulated in cancer cells. Samples include cultured pluripotent stem cells (four hES cell lines and one iPS cell line); 40 diverse clonal embryonic progenitor cell types including progenitors that are fully-differentiated while retaining a pre-EFT phenotype; 91 diverse cultured human cell types from all three germ layers; 18 diverse cultured epithelial cell types; 39 diverse sarcoma cell lines from diverse tissues; 35 diverse carcinoma and adenocarcinoma cell lines originating from diverse tissues; and four blood cell cancer lines. FIG. 8B) Scatter plot of COX7A1 vs MANEAL expression in 35 diverse carcinoma and adenocarcinoma cell lines originating from diverse tissues. FIG. 8C) Down-regulation of MANEAL in embryonic progenitors and cancer cells following expression of the segmental iTM and iCM factor COX7A1.

FIG. 9. Up-Regulation of the Maturation Marker COX7A1 in diverse cell types following 14 days of exposure to T3 Hormone. HBVSMC are Human Brain Vascular Smooth Muscle Cells. MDW are dermal fibroblasts. HAoSMCs are Aortic Smooth Muscle Cells. Error bars represent standard deviation.

### DETAILED DESCRIPTION

### Abbreviations

AC—Adult-derived cells
AMH—Anti-Mullerian Hormone
ASC—Adult stem cells
cGMP—Current Good Manufacturing Processes
CM—Cancer Maturation
CNS—Central Nervous System
CSC—Cancer Stem Cells
DMEM—Dulbecco's modified Eagle's medium
DMSO—Dimethyl sulphoxide
DNAm—Changes in the methylation of DNA that provide a marker or “clock” of the age of cells and tissue.
DPBS—Dulbecco's Phosphate Buffered Saline
DR-iTR—Developmentally-Regulated induced Tissue Regeneration
DR-O—Developmentally-Regulated Oncolysis
ED Cells—Embryo-derived cells; hED cells are human ED cells
EDTA—Ethylenediamine tetraacetic acid
EFT—Embryonic-Fetal Transition
EG Cells—Embryonic germ cells; hEG cells are human EG cells
EP—Embryonic progenitors
ES Cells—Embryonic stem cells; hES cells are human ES cells
ESC—Embryonic Stem Cells
FACS—Fluorescence activated cell sorting
FBS—Fetal bovine serum
FPKM—Fragments Per Kilobase of transcript per Million mapped reads from RNA sequencing.
GFER—Growth Factor, Augmenter of Liver Regeneration (ALR)
GFP—Green fluorescent protein
GMP—Good Manufacturing Practices
HAEC—Human Aortic Endothelial Cell
hED Cells—Human embryo-derived cells
hEG Cells—Human embryonic germ cells are stem cells derived from the primordial germ cells of fetal tissue.
HESC—Human Embryonic Stem Cells
hiPS Cells—Human induced pluripotent stem cells are cells with properties similar to hES cells obtained from somatic cells after exposure to hES-specific transcription factors such as SOX2, KLF4, OCT4, MYC, or NANOG, LIN28, OCT4, and SOX2.
HSE—Human skin equivalents are mixtures of cells and biological or synthetic matrices manufactured for testing purposes or for therapeutic application in promoting wound repair.
iCM—Induced Cancer Maturation.
IGF2—Insulin-like growth factor 2
iPS Cells—Induced pluripotent stem cells are cells with properties similar to hES cells obtained from somatic cells after exposure to ES-specific transcription factors such as SOX2, KLF4, OCT4, MYC, or NANOG, LIN28, OCT4, and SOX2, SOX2, KLF4, OCT4, MYC, and (LIN28A or LIN28B), or other combinations of OCT4, SOX2, KLF4, NANOG, ESRRB, NR5A2, CEBPA, MYC, LIN28A and LIN28B.
IRES—Internal Ribosome Entry Site
iS-CSC—induced Senolysis of Cancer Stem Cells refers to the treatment of cells in malignant tumors that are refractory to ablation by chemotherapeutic agents or radiation therapy wherein said iS-CSC treatment causes said refractory cells to revert to a pre-fetal pattern of gene expression and become sensitive to chemotherapeutic agents or radiation therapy.
iTM—Induced Cell and Tissue Maturation
iTR—Induced Tissue Regeneration
MEM—Minimal essential medium
MSC—Mesenchymal stem cell
NT—Nuclear Transfer
PBS—Phosphate buffered saline
POMC—Proopiomelanocortin
PPT—Prenatal-Postnatal Transition refers to the molecular alterations that occur in cells of placental mammals at or within a week of birth.
PS fibroblasts—Pre-scarring fibroblasts are fibroblasts derived from the skin of early gestational skin or derived from ED cells that display a prenatal pattern of gene expression in that they promote the rapid healing of dermal wounds without scar formation.
RFU—Relative Fluorescence Units
RNA-seq—RNA sequencing
SFM—Serum-Free Medium
St. Dev. —Standard Deviation
TR—Tissue Regeneration

### Definitions

The term “analytical reprogramming technology” refers to a variety of methods to reprogram the pattern of gene expression of a somatic cell to that of a more pluripotent state, such as that of an iPS, ES, ED, EC or EG cell, wherein the reprogramming occurs in multiple and discrete steps and does not rely simply on the transfer of a somatic cell into an oocyte and the activation of that oocyte (see U.S. application nos. 60/332,510, filed Nov. 26, 2001; Ser. No. 10/304,020, filed Nov. 26, 2002; PCT application no. PCT/US02/37899, filed Nov. 26, 2003; U.S. application No. 60/705,625, filed Aug. 3, 2005; U.S. application No. 60/729,173, filed Aug. 20, 2005; U.S. application No. 60/818,813, filed Jul. 5, 2006, PCT/US06/30632, filed Aug. 3, 2006, the disclosure of each of which is incorporated by reference herein).

The term “blastomere/morula cells” refers to blastomere or morula cells in a mammalian embryo or blastomere or morula cells cultured in vitro with or without additional cells including differentiated derivatives of those cells.

The term “Cancer Stem Cells” (CSC) refers to cells referenced in the art as cells with increased capacity of metastasis, relative resistance to chemo- or radiotherapy, and a more mesenchymal phenotype. As previously disclosed, the inventors of the present invention teach that contrary to common belief, CSCs are not a more developmentally immature cell, but instead are cancer cells with an adult pattern of gene expression while the cancer cells other than the CSCs are cancer cells with an embryonic (pre-fetal) pattern of gene expression.

The term “cell expressing gene X”, “gene X is expressed in a cell” (or cell population), or equivalents thereof, means that analysis of the cell using a specific assay platform provided a positive result. The converse is also true (i.e., by a cell not expressing gene X, or equivalents, is meant that analysis of the cell using a specific assay platform provided a negative result). Thus, any gene expression result described herein is tied to the specific probe or probes employed in the assay platform (or platforms) for the gene indicated.

The term “cell line” refers to a mortal or immortal population of cells that is capable of propagation and expansion in vitro.

The term “clonal” refers to a population of cells obtained the expansion of a single cell into a population of cells all derived from that original single cells and not containing other cells.

The term “differentiated cells” when used in reference to cells made by methods of this invention from pluripotent stem cells refer to cells having reduced potential to differentiate when compared to the parent pluripotent stem cells. The differentiated cells of this invention comprise cells that could differentiate further (i.e., they may not be terminally differentiated). Furthermore, the terms “differentiated” or “differentiated cells” refers to cells that display markers unique to the diverse somatic cell types such at cardiac troponin (TNNI3) in the cases of heart muscle cells or MYOD1 and MYOG in the case of skeletal muscle cell lineages, however, the terms “differentiated” or “differentiated cells” is distinct from the term “maturation” as used herein.

The term “embryonic” or “embryonic stages of development” refers to prenatal stages of development of cells, tissues or animals, specifically, the embryonic phases of development of cells compared to fetal and adult cells. In the case of the human species, the transition from embryonic to fetal development occurs at about 8 weeks of prenatal development, in mouse it occurs on or about 16 days, and in the rat species, at approximately 17.5 days post coitum. (www.php.med.unsw.edu.au/embryology/index.php?title=Mouse_Timeline_Detailed).

The term “embryonic-fetal transition” or “EFT” refers to the point in mammalian prenatal development wherein cells transition from the embryonic phases of development of cells to that of fetal cells. In the case of the human species, the transition from embryonic to fetal development occurs at about 8 weeks of prenatal development, in mouse it occurs on or about 16 days, and in the rat species, at approximately 17.5 days post coitum.

The term “embryonic stem cells” (ES cells) refers to cells derived from the inner cell mass of blastocysts, blastomeres, or morulae that have been serially passaged as cell lines while maintaining an undifferentiated state (e.g. expressing TERT, OCT4, and SSEA and TRA antigens specific for ES cells of the species). The ES cells may be derived from fertilization of an egg cell with sperm or DNA, nuclear transfer, parthenogenesis, or by means to generate hES cells with hemizygosity or homozygosity in the MHC region. While ES cells have historically been defined as cells capable of differentiating into all of the somatic cell types as well as germ line when transplanted into a preimplantation embryo, candidate ES cultures from many species, including human, have a more flattened appearance in culture and typically do not contribute to germ line differentiation, and are therefore called “ES-like cells.” It is commonly believed that human ES cells are in reality “ES-like”, however, in this application we will use the term ES cells to refer to both ES and ES-like cell lines.

The term “global modulator of TR” or “global modulator of iTR” refers to agents capable of modulating a multiplicity of iTR genes or iTM genes including, but not limited to, agents capable of downregulating COX7A1 while simultaneously up-regulating PCDHB2, or down-regulating NAALADL1 while simultaneously up-regulating AMH in cells derived from fetal or adult sources and are capable of inducing a pattern of gene expression leading to increased scarless tissue regeneration in response to tissue damage or degenerative disease. The term “global TR” or “global iTR” refers to when fetal or adult cells are induced to express a gene expression pattern similar to embryonic (pre-fetal) cells.

The terms “Global Regulator of cell and tissue maturation” and “Global regulator of cancer maturation” refer to agents that mature non-cancerous embryonic (pre-fetal) or cancer cells with an embryonic pattern of gene expression respectively to that of a later fetal or adult cell as determined by a plurality of adult cell markers being expressed at levels comparable to later fetal or adult cells. A Global Regulator of cell and tissue maturation is therefore contrasted with a Segmental regulator of cell and tissue maturation or of cancer cell maturation wherein only a single gene expression pattern, or a minority of gene expression markers characteristic of later fetal or adult cells is induced. The term “global cell or tissue maturation” or “global cancer maturation” refers to methods when non-cancerous embryonic (pre-fetal) or cancer cells with an embryonic pattern of gene expression are induced to express a gene expression pattern similar to later fetal or adult cells. The term “segmental cell or tissue maturation” or “segmental cancer maturation” refers to methods when non-cancerous embryonic (pre-fetal) or cancer cells with an embryonic pattern of gene expression are induced to express one are a few of the gene expressed in later fetal or adult cells.

The term “human embryo-derived” (“hED”) cells refers to blastomere-derived cells, morula-derived cells, blastocyst-derived cells including those of the inner cell mass, embryonic shield, or epiblast, or other totipotent or pluripotent stem cells of the early embryo, including primitive endoderm, ectoderm, mesoderm, and neural crest and their derivatives up to a state of differentiation correlating to the equivalent of the first eight weeks of normal human development, but excluding cells derived from hES cells that have been passaged as cell lines (see, e.g., U.S. Pat. Nos. 7,582,479; 7,217,569; 6,887,706; 6,602,711; 6,280,718; and U.S. Pat. No. 5,843,780 to Thomson). The hED cells may be derived from preimplantation embryos produced by fertilization of an egg cell with sperm or DNA, nuclear transfer, or chromatin transfer, an egg cell induced to form a parthenote through parthenogenesis, analytical reprogramming technology, or by means to generate hES cells with hemizygosity or homozygosity in the HLA region.

The term “human embryonic germ cells” (hEG cells) refer to pluripotent stem cells derived from the primordial germ cells of fetal tissue or maturing or mature germ cells such as oocytes and spermatogonial cells, that can differentiate into various tissues in the body. The hEG cells may also be derived from pluripotent stem cells produced by gynogenetic or androgenetic means, i.e., methods wherein the pluripotent cells are derived from oocytes containing only DNA of male or female origin and therefore will comprise all female-derived or male-derived DNA.

The term “human embryonic stem cells” (hES cells) refers to human ES cells.

The term “human induced pluripotent stem cells” refers to cells with properties similar to hES cells, including the ability to form all three germ layers when transplanted into immunocompromised mice wherein said iPS cells are derived from cells of varied somatic cell lineages following exposure to de-differentiation factors, for example hES cell-specific transcription factor combinations: KLF4, SOX2, MYC; OCT4 or SOX2, OCT4, NANOG, and LIN28; or various combinations of OCT4, SOX2, KLF4, NANOG, ESRRB, NR5A2, CEBPA, MYC, LIN28A and LIN28B or other methods that induce somatic cells to attain a pluripotent stem cell state with properties similar to hES cells. However, the reprogramming of somatic cells by somatic cell nuclear transfer (SCNT) are typically referred to as NT-ES cells as opposed to iPS cells.

The term “induced Cancer Maturation” refers to methods resulting in a change in the phenotype of premalignant or malignant cells such that subsequent to said induction, the cells express markers normally expressed in that cell type in fetal or adult stages of development as opposed to the embryonic stages.

The term “induced Senolysis of Cancer Stem Cells” (iS-CSC) refers to the treatment of cells in malignant tumors that are refractory to ablation by chemotherapeutic agents or radiation therapy wherein said iS-CSC treatment causes said refractory cells to revert to a pre-fetal pattern of gene expression and become sensitive to chemotherapeutic agents or radiation therapy.

The term “induced Tissue Maturation” or alternatively, the term “induced Cell and Tissue Maturation” refers to the advancement of the maturation of mammalian cells from an embryonic (pre-fetal) pattern of gene expression to one of a later fetal or adult pattern of gene expression.

The term “induced tissue regeneration” refers to the use of the methods of the present invention to alter the molecular composition of fetal or adult mammalian cells such that said cells are capable or regenerating functional tissue following damage to that tissue wherein said regeneration would not be the normal outcome in animals of that species. While functionally iTR is intended to generate new tissue formation at the sights of injury or degenerative disease or to induce senolysis in CSCs or aged cells, the inventors of the present invention teach that in iTR is in fact reversing many aspects of aging in cells including markers such as DNAm but not restoring telomerase activity. The addition of telomerase activity together with iTR is also defined in the present invention as “iTR”.

The term “iTR-related Senolysis” refers to the induction of apoptosis in cells of aged tissues that have significant DNA damage including but not limited to that from cell aging (telomere shortening) through the reprogramming of said damaged cells to an embryonic pattern of gene expression.

The term “isolated” refers to a substance that is (i) separated from at least some other substances with which it is normally found in nature, usually by a process involving the hand of man, (ii) artificially produced (e.g., chemically synthesized), and/or (iii) present in an artificial environment or context (i.e., an environment or context in which it is not normally found in nature).

The term “iS-CSC factors” refers to molecules that alter the levels of TR activators and TR inhibitors in a manner leading to TR and associated increase in sensitivity to apoptosis of cancer cells exposed to chemotherapeutic or radiation therapy.

The term “iCM factor” refers to any small molecule, protein, nucleic acid, or other molecules that when used singly or in combination with other molecules induce cancer cell maturation (induced Cancer Maturation).

The term “iTM factor” refers to any small molecule, protein, nucleic acid, or other molecules that when used singly or in combination with other molecules induce the maturation of cells with an embryonic phenotype (induced Cell and Tissue Maturation).

The term “iTR factor” refers to molecules that alter the levels of TR activators and TR inhibitors in a manner leading to TR in a tissue not naturally capable of TR. Said iTR factor also refers to combinations of individual factors. Therefore cocktails of factors described herein including but not limited to the cocktail designated AgeX1547 is considered an “iTR factor” in the present application.

The term “iTR genes” refers to genes that when altered in expression can cause induced tissue regeneration in tissues not normally capable of such regeneration.

The term “maturation” as used herein, such as in the term “induced cell and tissue maturation” or “induced cancer maturation” refers to the process of transversing any somatic cell from an embryonic (pre-fetal) pattern of gene expression wherein the tissue in which said cells reside is no longer capable of scarless tissue regeneration and by way of nonlimiting example, marked by low to no expression of the gene COX7A1. The term “maturation” as used herein is not synonymous with differentiation. Cells may be fully differentiated, such as at the end of embryonic development but immediately prior to fetal development, but not mature, and as a result, said cells are capable of scarless tissue regeneration while in the pre-fetal state.

The term “nucleic acid” is used interchangeably with “polynucleotide” and encompasses in various embodiments naturally occurring polymers of nucleosides, such as DNA and RNA, and non-naturally occurring polymers of nucleosides or nucleoside analogs. In some embodiments a nucleic acid comprises standard nucleosides (abbreviated A, G, C, T, U). In other embodiments a nucleic acid comprises one or more non-standard nucleosides. In some embodiments, one or more nucleosides are non-naturally occurring nucleosides or nucleotide analogs. A nucleic acid can comprise modified bases (for example, methylated bases), modified sugars (2′-fluororibose, arabinose, or hexose), modified phosphate groups or other linkages between nucleosides or nucleoside analogs (for example, phosphorothioates or 5′-N-phosphoramidite linkages), locked nucleic acids, or morpholinos. In some embodiments, a nucleic acid comprises nucleosides that are linked by phosphodiester bonds, as in DNA and RNA. In some embodiments, at least some nucleosides are linked by non-phosphodiester bond(s). A nucleic acid can be single-stranded, double-stranded, or partially double-stranded. An at least partially double-stranded nucleic acid can have one or more overhangs, e.g., 5′ and/or 3′ overhang(s). Nucleic acid modifications (e.g., nucleoside and/or backbone modifications, including use of non-standard nucleosides) known in the art as being useful in the context of RNA interference (RNAi), aptamer, or antisense-based molecules for research or therapeutic purposes are contemplated for use in various embodiments of the instant invention. See, e.g., Crooke, S T (ed.) Antisense drug technology: principles, strategies, and applications, Boca Raton: CRC Press, 2008; Kurreck, J. (ed.) Therapeutic oligonucleotides, RSC biomolecular sciences. Cambridge: Royal Society of Chemistry, 2008. In some embodiments, a modification increases half-life and/or stability of a nucleic acid, e.g., in vivo, relative to RNA or DNA of the same length and strandedness. In some embodiments, a modification decreases immunogenicity of a nucleic acid relative to RNA or DNA of the same length and strandedness. In some embodiments, between 5% and 95% of the nucleosides in one or both strands of a nucleic acid is modified. Modifications may be located uniformly or nonuniformly, and the location of the modifications (e.g., near the middle, near or at the ends, alternating, etc.) can be selected to enhance desired propert(ies). A nucleic acid may comprise a detectable label, e.g., a fluorescent dye, radioactive atom, etc. “Oligonucleotide” refers to a relatively short nucleic acid, e.g., typically between about 4 and about 60 nucleotides long. Where reference is made herein to a polynucleotide, it is understood that both DNA, RNA, and in each case both single- and double-stranded forms (and complements of each single-stranded molecule) are provided.

“Polynucleotide sequence” as used herein can refer to the polynucleotide material itself and/or to the sequence information (i.e. the succession of letters used as abbreviations for bases) that biochemically characterizes a specific nucleic acid. A polynucleotide sequence presented herein is presented in a 5′ to 3′ direction unless otherwise indicated.

The term “pluripotent stem cells” refers to animal cells capable of differentiating into more than one differentiated cell type. Such cells include hES cells, blastomere/morula cells and their derived hED cells, hiPS cells, hEG cells, hEC cells, and adult-derived cells including mesenchymal stem cells, neuronal stem cells, and bone marrow-derived stem cells. Pluripotent stem cells may be genetically modified or not genetically modified. Genetically modified cells may include markers such as fluorescent proteins to facilitate their identification within the egg.

The term “polypeptide” refers to a polymer of amino acids. The terms “protein” and “polypeptide” are used interchangeably herein. A peptide is a relatively short polypeptide, typically between about 2 and 60 amino acids in length. Polypeptides used herein typically contain the standard amino acids (i.e., the 20 L-amino acids that are most commonly found in proteins). However, a polypeptide can contain one or more non-standard amino acids (which may be naturally occurring or non-naturally occurring) and/or amino acid analogs known in the art in certain embodiments. One or more of the amino acids in a polypeptide may be modified, for example, by the addition of a chemical entity such as a carbohydrate group, a phosphate group, a fatty acid group, a linker for conjugation, functionalization, etc. A polypeptide that has a nonpolypeptide moiety covalently or noncovalently associated therewith is still considered a “polypeptide”. Polypeptides may be purified from natural sources, produced using recombinant DNA technology, synthesized through chemical means such as conventional solid phase peptide synthesis, etc. The term “polypeptide sequence” or “amino acid sequence” as used herein can refer to the polypeptide material itself and/or to the sequence information (i.e., the succession of letters or three letter codes used as abbreviations for amino acid names) that biochemically characterizes a polypeptide. A polypeptide sequence presented herein is presented in an N-terminal to C-terminal direction unless otherwise indicated. A polypeptide may be cyclic or contain a cyclic portion. Where a naturally occurring polypeptide is discussed herein, it will be understood that the invention encompasses embodiments that relate to any isoform thereof (e.g., different proteins arising from the same gene as a result of alternative splicing or editing of mRNA or as a result of different alleles of a gene, e.g., alleles differing by one or more single nucleotide polymorphisms (typically such alleles will be at least 95%, 96%, 97%, 98%, 99%, or more identical to a reference or consensus sequence). A polypeptide may comprise a sequence that targets it for secretion or to a particular intracellular compartment (e.g., the nucleus) and/or a sequence targets the polypeptide for post-translational modification or degradation. Certain polypeptides may be synthesized as a precursor that undergoes post-translational cleavage or other processing to become a mature polypeptide. In some instances, such cleavage may only occur upon particular activating events. Where relevant, the invention provides embodiments relating to precursor polypeptides and embodiments relating to mature versions of a polypeptide.

The term “pre-fetal” refers to mammalian somatic cells in a stage of development corresponding to the same undifferentiated or differentiated cell type in the developing mammal before the embryonic-fetal transition (EFT).

The term “prenatal” refers to a stage of embryonic development of a placental mammal prior to which an animal is not capable of viability apart from the uterus.

The term “primordial stem cells” refers collectively to pluripotent stem cells capable of differentiating into cells of all three primary germ layers: endoderm, mesoderm, and ectoderm, as well as neural crest. Therefore, examples of primordial stem cells would include but not be limited by human or non-human mammalian ES cells or cell lines, blastomere/morula cells and their derived ED cells, iPS, and EG cells.

The term “purified” refers to agents or entities (e.g., compounds) that have been separated from most of the components with which they are associated in nature or when originally generated. In general, such purification involves action of the hand of man. Purified agents or entities may be partially purified, substantially purified, or pure. Such agents or entities may be, for example, at least 50%, 60%, 70%, 75%, 80%, 85%, 90%, 95%, 96%, 97%, 98%, 99%, or more than 99% pure. In some embodiments, a nucleic acid or polypeptide is purified such that it constitutes at least 75%, 80%, 855%, 90%, 95%, 96%, 97%, 98%, 99%, or more, of the total nucleic acid or polypeptide material, respectively, present in a preparation. Purity can be based on, e.g., dry weight, size of peaks on a chromatography tracing, molecular abundance, intensity of bands on a gel, or intensity of any signal that correlates with molecular abundance, or any art-accepted quantification method. In some embodiments, water, buffers, ions, and/or small molecules (e.g., precursors such as nucleotides or amino acids), can optionally be present in a purified preparation. A purified molecule may be prepared by separating it from other substances (e.g., other cellular materials), or by producing it in such a manner to achieve a desired degree of purity. In some embodiments, a purified molecule or composition refers to a molecule or composition that is prepared using any art-accepted method of purification. In some embodiments “partially purified” means that a molecule produced by a cell is no longer present within the cell, e.g., the cell has been lysed and, optionally, at least some of the cellular material (e.g., cell wall, cell membrane(s), cell organelle(s)) has been removed.

The term “RNA interference” (RNAi) is used herein consistently with its meaning in the art to refer to a phenomenon whereby double-stranded RNA (dsRNA) triggers the sequence-specific degradation or translational repression of a corresponding mRNA having complementarity to a strand of the dsRNA. It will be appreciated that the complementarity between the strand of the dsRNA and the mRNA need not be 100% but need only be sufficient to mediate inhibition of gene expression (also referred to as “silencing” or “knockdown”). For example, the degree of complementarity is such that the strand can either (i) guide cleavage of the mRNA in the RNA-induced silencing complex (RISC); or (ii) cause translational repression of the mRNA. In certain embodiments the double-stranded portion of the RNA is less than about 30 nucleotides in length, e.g., between 17 and 29 nucleotides in length. In certain embodiments a first strand of the dsRNA is at least 80%, 85%, 90%, 95%, or 100% complementary to a target mRNA and the other strand of the dsRNA is at least 80%, 85%, 90%, 95%, or 100% complementary to the first strand. In mammalian cells, RNAi may be achieved by introducing an appropriate double-stranded nucleic acid into the cells or expressing a nucleic acid in cells that is then processed intracellularly to yield dsRNA therein. Nucleic acids capable of mediating RNAi are referred to herein as “RNAi agents”. Exemplary nucleic acids capable of mediating RNAi are a short hairpin RNA (shRNA), a short interfering RNA (siRNA), and a microRNA precursor. These terms are well known and are used herein consistently with their meaning in the art. siRNAs typically comprise two separate nucleic acid strands that are hybridized to each other to form a duplex. They can be synthesized in vitro, e.g., using standard nucleic acid synthesis techniques. siRNAs are typically double-stranded oligonucleotides having 16-30, e.g., 16, 17, 18, 19, 20, 21, 22, 23, 24, 25, 26, 27, 28, 29, or 30 nucleotides (nt) in each strand, wherein the double-stranded oligonucleotide comprises a double-stranded portion between 15 and 29 nucleotides long and either or both of the strands may comprise a 3′ overhang between, e.g., 1-5 nucleotides long, or either or both ends can be blunt. In some embodiments, an siRNA comprises strands between 19 and 25 nt, e.g., between 21 and 23 nucleotides long, wherein one or both strands comprises a 3′ overhang of 1-2 nucleotides. One strand of the double-stranded portion of the siRNA (termed the “guide strand” or “antisense strand”) is substantially complementary (e.g., at least 80% or more, e.g., 85%, 90%, 95%, or 100%) complementary to (e.g., having 3, 2, 1, or 0 mismatched nucleotide(s)) a target region in the mRNA, and the other double-stranded portion is substantially complementary to the first double-stranded portion. In many embodiments, the guide strand is 100% complementary to a target region in an mRNA and the other passenger strand is 100% complementary to the first double-stranded portion (it is understood that, in various embodiments, the 3′ overhang portion of the guide strand, if present, may or may not be complementary to the mRNA when the guide strand is hybridized to the mRNA). In some embodiments, a shRNA molecule is a nucleic acid molecule comprising a stem-loop, wherein the double-stranded stem is 16-30 nucleotides long and the loop is about 1-10 nucleotides long. siRNA can comprise a wide variety of modified nucleosides, nucleoside analogs and can comprise chemically or biologically modified bases, modified backbones, etc. Without limitation, any modification recognized in the art as being useful for RNAi can be used. Some modifications result in increased stability, cell uptake, potency, etc. Some modifications result in decreased immunogenicity or clearance. In certain embodiments the siRNA comprises a duplex about 19-23 (e.g., 19, 20, 21, 22, or 23) nucleotides in length and, optionally, one or two 3′ overhangs of 1-5 nucleotides in length, which may be composed of deoxyribonucleotides. shRNA comprise a single nucleic acid strand that contains two complementary portions separated by a predominantly non-selfcomplementary region. The complementary portions hybridize to form a duplex structure and the non-selfcomplementary region forms a loop connecting the 3′ end of one strand of the duplex and the 5′ end of the other strand. shRNAs undergo intracellular processing to generate siRNAs. Typically, the loop is between 1 and 8, e.g., 2-6 nucleotides long.

MicroRNAs (miRNAs) are small, naturally occurring, non-coding, single-stranded RNAs of about 21-25 nucleotides (in mammalian systems) that inhibit gene expression in a sequence-specific manner. They are generated intracellularly from precursors (pre-miRNA) having a characteristic secondary structure comprised of a short hairpin (about 70 nucleotides in length) containing a duplex that often includes one or more regions of imperfect complementarity which is in turn generated from a larger precursor (pri-miRNA). Naturally occurring miRNAs are typically only partially complementary to their target mRNA and often act via translational repression. RNAi agents modelled on endogenous miRNA or miRNA precursors are of use in certain embodiments of the invention. For example, an siRNA can be designed so that one strand hybridizes to a target mRNA with one or more mismatches or bulges mimicking the duplex formed by a miRNA and its target mRNA. Such siRNA may be referred to as miRNA mimics or miRNA-like molecules. miRNA mimics may be encoded by precursor nucleic acids whose structure mimics that of naturally occurring miRNA precursors.

In certain embodiments an RNAi agent is a vector (e.g., a plasmid or virus) that comprises a template for transcription of an siRNA (e.g., as two separate strands that can hybridize to each other), shRNA, or microRNA precursor. Typically the template encoding the siRNA, shRNA, or miRNA precursor is operably linked to expression control sequences (e.g., a promoter), as known in the art. Such vectors can be used to introduce the template into vertebrate cells, e.g., mammalian cells, and result in transient or stable expression of the siRNA, shRNA, or miRNA precursor. Precursors (shRNA or miRNA precursors) are processed intracellularly to generate siRNA or miRNA.

In general, small RNAi agents such as siRNA can be chemically synthesized or can be transcribed in vitro or in vivo from a DNA template either as two separate strands that then hybridize, or as an shRNA which is then processed to generate an siRNA. Often RNAi agents, especially those comprising modifications, are chemically synthesized. Chemical synthesis methods for oligonucleotides are well known in the art.

The term “small molecule” as used herein, is an organic molecule that is less than about 2 kilodaltons (KDa) in mass. In some embodiments, the small molecule is less than about 1.5 KDa, or less than about 1 KDa. In some embodiments, the small molecule is less than about 800 daltons (Da), 600 Da, 500 Da, 400 Da, 300 Da, 200 Da, or 100 Da. Often, a small molecule has a mass of at least 50 Da. In some embodiments, a small molecule contains multiple carbon-carbon bonds and can comprise one or more heteroatoms and/or one or more functional groups important for structural interaction with proteins (e.g., hydrogen bonding), e.g., an amine, carbonyl, hydroxyl, or carboxyl group, and in some embodiments at least two functional groups. Small molecules often comprise one or more cyclic carbon or heterocyclic structures and/or aromatic or polyaromatic structures, optionally substituted with one or more of the above functional groups. In some embodiments, a small molecule is non-polymeric. In some embodiments, a small molecule is not an amino acid. In some embodiments, a small molecule is not a nucleotide. In some embodiments, a small molecule is not a saccharide.

As used herein, the term “somatic cell” refers to cells that are differentiated or partially-differentiated (such as embryonic progenitors derived from pluripotent stem cell. Somatic cell include but are not limited to clonal embryonic progenitor cell lines) include, but are not limited to: cardiac cells, stomach cells, neural cells, lung cells, cells of the ear, cells of the olfactory system, reproductive cells, pancreatic cells, gastrointestinal cells, thyroid cells, epithelial cells, bladder cells, blood cells, respiratory tract cells, salivary gland cells, adipocytes, cells of the eye, liver cells, muscle cells, kidney cells, and immune system cells.

According to come embodiments, the somatic cell is derived from endoderm germ layer such as primitive foregut, midgut, and hindgut endoderm.

According to come embodiments, the somatic cell is derived from hepatocytes.

According to come embodiments, the somatic cell is derived from exocrine secretory epithelial cells.

According to come embodiments, the somatic cell is derived from Brunner's gland cell in duodenum.

According to come embodiments, the somatic cell is derived from insulated goblet cell of respiratory or digestive tracts.

According to come embodiments, the somatic cell is derived from cells of the stomach, such as foveolar, chief, and parietal cells.

According to come embodiments, the somatic cell is derived from pancreatic acinar cells.

According to come embodiments, the somatic cell is derived from paneth cell of small intestine.

According to come embodiments, the somatic cell is derived from lung cells, such as type I pneumocytes of the lung, type II pneumocytes of the lung, club cells of the lung.

According to come embodiments, the somatic cell is derived from barrier cells.

According to come embodiments, the somatic cell is derived from gall bladder epithelial cells.

According to come embodiments, the somatic cell is derived from the pancreas, such as pancreatic centroacinar cells, pancreatic intercalated duct cells, pancreatic islet cells of the islets of Langerhans including: alpha cells, beta cells, delta cells, epsilon cells, and PP cells (also known as gamma cells).

According to come embodiments, the somatic cell is derived from cells of the gastrointestinal tract, such as intestinal brush border cells; enteroendocrine cells, e.g., K cells, L cells, I cells, G cells, enterochromaffin cells, enterochromaffin-like cells, N cells, S cells, D cells, and M cells.

According to come embodiments, the somatic cell is derived from cells of the thyroid or parathyroid, such as thyroid gland cells, thyroid epithelial cells parafollicular cells, parathyroid gland cells, parathyroid chief cells, and oxyphil cells.

According to come embodiments, the somatic cell is derived from cells of the ectoderm germ layer such as neuroepithelial cells, neural crest cells, and ectoderm-derived exocrine secretory epithelial cells.

According to come embodiments, the somatic cell is derived from cells of the salivary gland, such as salivary gland mucous cells, salivary gland serous cells, and Von Ebner's gland cell of the tongue.

According to come embodiments, the somatic cell is derived from mammary gland cells.

According to come embodiments, the somatic cell is derived from lacrimal gland cells.

According to come embodiments, the somatic cell is derived from ceruminous gland cell in ear.

According to come embodiments, the somatic cell is derived from eccrine sweat gland dark cells.

According to come embodiments, the somatic cell is derived from eccrine sweat gland clear cell.

According to come embodiments, the somatic cell is derived from apocrine sweat gland cell.

According to come embodiments, the somatic cell is derived from gland of Moll cell in eyelid.

According to come embodiments, the somatic cell is derived from sebaceous gland cells.

According to come embodiments, the somatic cell is derived from Bowman's gland cell of the nose.

According to come embodiments, the somatic cell is derived from hormone-secreting cells including but not limited to: anterior/intermediate pituitary cells such as: corticotropes, gonadotropes, lactotropes, melanotropes, somatotropes, and thyrotropes.

According to come embodiments, the somatic cell is derived from magnocellular neurosecretory cells.

According to come embodiments, the somatic cell is derived from parvocellular neurosecretory cells.

According to come embodiments, the somatic cell is derived from chromaffin cells of the adrenal gland.

According to come embodiments, the somatic cell is derived from diverse ectoderm-derived epithelial cells including: periderm and stratified keratinocytes.

According to come embodiments, the somatic cell is derived from epidermal basal cells.

According to come embodiments, the somatic cell is derived from melanocytes.

According to come embodiments, the somatic cell is derived from trichocytes.

According to come embodiments, the somatic cell is derived from cells of hair or hair shaft, such and medullary hair shaft cells, cortical hair shaft cells, cuticular hair shaft cells, Huxley's layer hair root sheath cells, Henle's layer hair root sheath cells, and outer root sheath hair cells.

According to come embodiments, the somatic cell is derived from surface epithelial cells of the cornea, tongue, mouth, nasal cavity, distal anal canal, distal urethra, and distal vagina.

According to come embodiments, the somatic cell is derived from basal cells (stem cells) of the cornea, tongue, mouth, nasal cavity, distal anal canal, distal urethra, and distal vagina.

According to come embodiments, the somatic cell is derived from intercalated duct cells of salivary glands or striated duct cells of salivary glands.

According to come embodiments, the somatic cell is derived from lactiferous duct cells of mammary glands.

According to come embodiments, the somatic cell is derived from cartilage progenitor cells of neural crest origin, such as ameloblasts, odontoblasts, and cementoblasts.

According to come embodiments, the somatic cell is derived from cells of the nervous system such as neural tube epithelial cells, neural progenitors of the forebrain, midbrain, hindbrain, and spinal cord.

According to come embodiments, the somatic cell is derived from radial glial cells.

According to come embodiments, the somatic cell is derived from sensory transducer cells.

According to come embodiments, the somatic cell is derived from auditory inner hair cells of the organ of Corti or auditory outer hair cells of organ of Corti.

According to come embodiments, the somatic cell is derived from basal cells of the olfactory epithelium.

According to come embodiments, the somatic cell is derived from cold-sensitive primary sensory neurons.

According to come embodiments, the somatic cell is derived from heat-sensitive primary sensory neurons.

According to come embodiments, the somatic cell is derived from Merkel cells of the epidermis.

According to come embodiments, the somatic cell is derived from olfactory receptor neurons.

According to come embodiments, the somatic cell is derived from pain-sensitive primary sensory neurons.

According to come embodiments, the somatic cell is derived from photoreceptor cells of retina in eye including: photoreceptor rod, blue-sensitive cone, green-sensitive cone, and red-sensitive cone cells of eye.

According to come embodiments, the somatic cell is derived from proprioceptive primary sensory neurons.

According to come embodiments, the somatic cell is derived from touch-sensitive primary sensory neurons.

According to come embodiments, the somatic cell is derived from chemoreceptor glomus cells of carotid body cell.

According to come embodiments, the somatic cell is derived from the ear, such as outer hair cells of vestibular system of ear, inner hair cells of vestibular system of ear inner, outer pillar cells of the organ of Corti, inner and outer phalangeal cells of the organ of Corti, border cells of the organ of Corti, and Hensen's cells of the organ of Corti.

According to come embodiments, the somatic cell is derived from taste receptor cells of taste bud.

According to come embodiments, the somatic cell is derived from autonomic neuron cells including: cholinergic, adrenergic, and peptidergic neural cells.

According to come embodiments, the somatic cell is derived from sense organ and peripheral neuron supporting cells including: outer pillar cells of the organ of Corti, inner and outer phalangeal cells of the organ of Corti, border cells of the organ of Corti, Hensen's cells of the organ of Corti, vestibular apparatus supporting cells, taste bud supporting cells, olfactory epithelium supporting cells, olfactory ensheathing cells, Schwann cells, satellite glial cells, and enteric glial cells.

According to come embodiments, the somatic cell is derived from central nervous system neurons and glial cells including: neuron cells, interneurons, basket cells, cartwheel cells, stellate cells, golgi cells, granule cells, Lugaro cells, unipolar brush cells, Martinotti cells, chandelier cells, Cajal-Retzius cells, double-bouquet cells, neurogliaform cells, retina horizontal cells, amacrine cells, starburst amacrine cells, spinal interneurons, Renshaw cells, principal cells, spindle neurons, fork neurons, pyramidal cells, place cells, grid cells, speed cells, head direction cells, betz cells, stellate cells, boundary cells, bushy cells, Purkinje cells, medium spiny neurons, astrocytes, oligodendrocytes, ependymal cells, tanycytes, pituicytes, lens cells, anterior lens epithelial cells, and crystallin-containing lens fiber cells.

According to come embodiments, the somatic cell is derived from cells derived primarily from mesoderm including: white, beige, and brown preadipocytes and adipocytes.

According to come embodiments, the somatic cell is derived from pericardial preadipocytes and adipocytes.

According to come embodiments, the somatic cell is derived from liver preadipocytes and adipocytes.

According to come embodiments, the somatic cell is derived from liver lipocytes.

According to come embodiments, the somatic cell is derived from cells of the adrenal cortex including: cells of the zona glomerulosa, cells of the zona fasciculata, and cells of the zona reticularis.

According to come embodiments, the somatic cell is derived from theca interna cells of the ovarian follicle.

According to come embodiments, the somatic cell is derived from theca interna cells of the ovarian follicle corpus luteum cells.

According to come embodiments, the somatic cell is derived from granulosa and theca lutein cells.

According to come embodiments, the somatic cell is derived from Leydig cells of the testes.

According to come embodiments, the somatic cell is derived from seminal vesicle cell.

According to come embodiments, the somatic cell is derived from prostate gland cells.

According to come embodiments, the somatic cell is derived from bulbourethral gland cells.

According to come embodiments, the somatic cell is derived from Bartholin's gland cells.

According to come embodiments, the somatic cell is derived from gland of Littre cell.

According to come embodiments, the somatic cell is derived from uterus endometrial cells.

According to come embodiments, the somatic cell is derived from juxtaglomerular cells producing renin.

According to come embodiments, the somatic cell is derived from macula densa cells of kidney.

According to come embodiments, the somatic cell is derived from cell of the peripolar cell of kidney.

According to come embodiments, the somatic cell is derived from mesangial cell of kidney.

According to come embodiments, the somatic cell is derived from urinary system cells including: parietal epithelial cells, podocytes, proximal tubule brush border cells, loop of Henle thin segment cells, kidney distal tubule cells, kidney collecting duct cells, principal cells, intercalated cells, and transitional epithelium (lining urinary bladder).

According to come embodiments, the somatic cell is derived from reproductive system cells including: duct cells of seminal vesicle, prostate gland epithelium, efferent duct cells, epididymal principal cells, and epididymal basal cells.

According to come embodiments, the somatic cell is derived from cells of circulatory system including: vascular endothelial cells, microvascular endothelial cells, microvascular endothelial cells of the brain; lymphatic endothelial cells, arterial endothelial cells, venous endothelial cells, vascular pericytes.

According to come embodiments, the somatic cell is derived from stromal fibroblasts residing in diverse tissues of the body.

According to come embodiments, the somatic cell is derived from planum semilunatum epithelial cell of vestibular system of ear.

According to come embodiments, the somatic cell is derived from organ of Corti interdental epithelial cell.

According to come embodiments, the somatic cell is derived from loose connective tissue fibroblasts.

According to come embodiments, the somatic cell is derived from corneal fibroblasts;

According to come embodiments, the somatic cell is derived from tendon fibroblasts.

According to come embodiments, the somatic cell is derived from bone marrow reticular tissue fibroblasts.

According to come embodiments, the somatic cell is derived from bone marrow mesenchymal stem cells.

According to come embodiments, the somatic cell is derived from mesenchymal stem cells derived from diverse tissues of the body.

According to come embodiments, the somatic cell is derived from hepatic stellate cells (Ito cells).

According to come embodiments, the somatic cell is derived from somite cells.

According to come embodiments, the somatic cell is derived from nucleus pulposus cell of the intervertebral disc and their progenitors.

According to come embodiments, the somatic cell is derived from hyaline cartilage progenitor cells of mesodermal origin and their progenitors.

According to come embodiments, the somatic cell is derived from hyaline cartilage chondrocytes and their progenitors.

According to come embodiments, the somatic cell is derived from fibrocartilage chondrocytes and their progenitors.

According to come embodiments, the somatic cell is derived from elastic cartilage chondrocytes and their progenitors.

According to come embodiments, the somatic cell is derived from osteoblasts and osteocytes and their progenitors.

According to come embodiments, the somatic cell is derived from osteoprogenitor cells.

According to come embodiments, the somatic cell is derived from stellate cell of the perilymphatic space of the ear.

According to come embodiments, the somatic cell is derived from pancreatic stellate cells.

According to come embodiments, the somatic cell is derived from contractile cells including: skeletal muscle myoblast cells, skeletal myocytes, red skeletal muscle cells, white skeletal muscle cell, intermediate skeletal muscle cells, and myosatellite cells.

According to come embodiments, the somatic cell is derived from cardiac muscle cells including: atrial and ventricular cardiac muscle progenitor and differentiated cells; cardiac SA node cells; Purkinje fiber cells, coronary artery cells, coronary artery smooth muscle cells; coronary artery vascular endothelial cells, and cardiac stromal fibroblast cells.

According to come embodiments, the somatic cell is derived from myoepithelial cell of the iris.

According to come embodiments, the somatic cell is derived from myoepithelial cell of exocrine glands.

According to come embodiments, the somatic cell is derived from blood and immune system cells including without limitation: hematopoietic vacular endothelium, hematopoietic stem cells and committed progenitors for the blood and immune system, hematopoietic stem cells of the fetal liver, erythroblasts, erythrocytes, megakaryocytes and platelets, monocytes, connective tissue macrophages; epidermal Langerhans cells, osteoclasts, dendritic cells, microglial cells, neutrophil granulocyte and precursors (myeloblasts, promyelocytes, myelocytes, metamyelocytes), eosinophils granulocytes and precursors, basophil granulocytes and precursors, mast cells, helper T-cells, regulatory T-cells, cytotoxic T-cells, natural killer T-cells, B-cells, plasma cells, and natural killer cells.

According to come embodiments, the somatic cell is derived from cells of the reproductive system including but not limited to: nurse cells, granulosa cells of the ovary, and Sertoli cells of the testis.

According to come embodiments, the somatic cell is derived from thymus epithelial reticular cells.

According to come embodiments, the somatic cell is derived from interstitial kidney cells.

According to come embodiments, the somatic cell is derived from contractile smooth muscle cells in diverse tissues of the body or vascular smooth muscle cells in diverse tissues of the body.

According to come embodiments, the somatic cell is derived from diverse connective tissue fibroblastic cells of the body.

According to come embodiments, the somatic cell is derived from splenocytes and reticular cells of the spleen.

The term “subject” can be any multicellular animal. Often a subject is a vertebrate, e.g., a mammal or avian. Exemplary mammals include, e.g., humans, non-human primates, rodents (e.g., mouse, rat, rabbit), ungulates (e.g., ovine, bovine, equine, caprine species), canines, and felines. Often, a subject is an individual to whom a compound is to be delivered, e.g., for experimental, diagnostic, and/or therapeutic purposes or from whom a sample is obtained or on whom a diagnostic procedure is performed (e.g., a sample or procedure that will be used to assess tissue damage and/or to assess the effect of a compound of the invention).

The term “tissue damage” is used herein to refer to any type of damage or injury to cells, tissues, organs, or other body structures. The term encompasses, in various embodiments, degeneration due to disease, damage due to physical trauma or surgery, damage caused by exposure to deleterious substance, and other disruptions in the structure and/or functionality of cells, tissues, organs, or other body structures.

The term “tissue regeneration” or “TR” refers to at least partial regeneration, replacement, restoration, or regrowth of a tissue, organ, or other body structure, or portion thereof, following loss, damage, or degeneration, where said tissue regeneration but for the methods described in the present invention would not take place. Examples of tissue regeneration include the regrowth of severed digits or limbs including the regrowth of cartilage, bone, muscle, tendons, and ligaments, the scarless regrowth of bone, cartilage, skin, or muscle that has been lost due to injury or disease, with an increase in size and cell number of an injured or diseased organ such that the tissue or organ approximates the normal size of the tissue or organ or its size prior to injury or disease. Depending on the tissue type, tissue regeneration can occur via a variety of different mechanisms such as, for example, the rearrangement of pre-existing cells and/or tissue (e.g., through cell migration), the division of adult somatic stem cells or other progenitor cells and differentiation of at least some of their descendants, and/or the dedifferentiation, transdifferentiation, and/or proliferation of cells.

The term “TR activator genes” refers to genes whose lack of expression in fetal and adult cells but whose expression in embryonic phases of development facilitate TR.

The term “TR inhibitor genes” refers to genes whose expression in fetal and adult animals inhibit TR.

The term “treat”, “treating”, “therapy”, “therapeutic” and similar terms in regard to a subject refer to providing medical and/or surgical management of the subject. Treatment can include, but is not limited to, administering a compound or composition (e.g., a pharmaceutical composition) to a subject. Treatment of a subject according to the instant invention is typically undertaken in an effort to promote regeneration, e.g., in a subject who has suffered tissue damage or is expected to suffer tissue damage (e.g., a subject who will undergo surgery). The effect of treatment can generally include increased regeneration, reduced scarring, and/or improved structural or functional outcome following tissue damage (as compared with the outcome in the absence of treatment), and/or can include reversal or reduction in severity or progression of a degenerative disease.

The term “variant” as applied to a particular polypeptide refers to a polypeptide that differs from such polypeptide (sometimes referred to as the “original polypeptide”) by one or more amino acid alterations, e.g., addition(s), deletion(s), and/or substitution(s). Sometimes an original polypeptide is a naturally occurring polypeptide (e.g., from human or non-human animal) or a polypeptide identical thereto. Variants may be naturally occurring or created using, e.g., recombinant DNA techniques or chemical synthesis. An addition can be an insertion within the polypeptide or an addition at the N- or C-terminus. In some embodiments, the number of amino acids substituted, deleted, or added can be for example, about 1 to 30, e.g., about 1 to 20, e.g., about 1 to 10, e.g., about 1 to 5, e.g., 1, 2, 3, 4, or 5. In some embodiments, a variant comprises a polypeptide whose sequence is homologous to the sequence of the original polypeptide over at least 50 amino acids, at least 100 amino acids, at least 150 amino acids, or more, up to the full length of the original polypeptide (but is not identical in sequence to the original polypeptide), e.g., the sequence of the variant polypeptide is at least 50%, 60%, 70%, 75%, 80%, 85%, 90%, 95%, 96%, 97%, 98%, 99%, or more identical to the sequence of the original polypeptide over at least 50 amino acids, at least 100 amino acids, at least 150 amino acids, or more, up to the full length of the original polypeptide. In some embodiments, a variant comprises a polypeptide at least 50%, 60%, 70%, 75%, 80%, 85%, 90%, 91%, 92%, 93%, 94%, 95%, 96%, 97%, 98%, 99%, 99.5% or more identical to an original polypeptide over at least 50%, 60%, 70%, 80%, 85%, 90%, 91%, 92%, 93%, 94%, 95%, 96%, 97%, 98%, 99%, or 100% of the length of the original polypeptide. In some embodiments a variant comprises at least one functional or structural domain, e.g., a domain identified as such in the Conserved Domain Database (CDD) of the National Center for Biotechnology Information (www.ncbi.nih.gov), e.g., an NCBI-curated domain.

In some embodiments one, more than one, or all biological functions or activities of a variant or fragment is substantially similar to that of the corresponding biological function or activity of the original molecule. In some embodiments, a functional variant retains at least 10%, 20%, 30%, 40%, 50%, 60%, 70%, 80%, 90%, 95%, 96%, 97%, 98%, 99%, or more of the activity of the original polypeptide, e.g., about equal activity. In some embodiments, the activity of a variant is up to approximately 100%, approximately 125%, or approximately 150% of the activity of the original molecule. In other nonlimiting embodiments an activity of a variant or fragment is considered substantially similar to the activity of the original molecule if the amount or concentration of the variant needed to produce a particular effect is within 0.5 to 5-fold of the amount or concentration of the original molecule needed to produce that effect.

In some embodiments amino acid “substitutions” in a variant are the result of replacing one amino acid with another amino acid having similar structural and/or chemical properties, i.e., conservative amino acid replacements. “Conservative” amino acid substitutions may be made on the basis of similarity in any of a variety or properties such as side chain size, polarity, charge, solubility, hydrophobicity, hydrophilicity, and/or amphipathicity of the residues involved. For example, the non-polar (hydrophobic) amino acids include alanine, leucine, isoleucine, valine, glycine, proline, phenylalanine, tryptophan and methionine. The polar (hydrophilic), neutral amino acids include serine, threonine, cysteine, tyrosine, asparagine, and glutamine. The positively charged (basic) amino acids include arginine, lysine and histidine. The negatively charged (acidic) amino acids include aspartic acid and glutamic acid. Within a particular group, certain substitutions may be of particular interest, e.g., replacements of leucine by isoleucine (or vice versa), serine by threonine (or vice versa), or alanine by glycine (or vice versa). Of course non-conservative substitutions are often compatible with retaining function as well. In some embodiments, a substitution or deletion does not alter or delete an amino acid important for activity. Insertions or deletions may range in size from about 1 to 20 amino acids, e.g., 1 to 10 amino acids. In some instances larger domains may be removed without substantially affecting function. In certain embodiments of the invention the sequence of a variant can be obtained by making no more than a total of 5, 10, 15, or 20 amino acid additions, deletions, or substitutions to the sequence of a naturally occurring enzyme. In some embodiments no more than 1%, 5%, 10%, or 20% of the amino acids in a polypeptide are insertions, deletions, or substitutions relative to the original polypeptide. Guidance in determining which amino acid residues may be replaced, added, or deleted without eliminating or substantially reducing activities of interest, may be obtained by comparing the sequence of the particular polypeptide with that of homologous polypeptides (e.g., from other organisms) and minimizing the number of amino acid sequence changes made in regions of high homology (conserved regions) or by replacing amino acids with those found in homologous sequences since amino acid residues that are conserved among various species are more likely to be important for activity than amino acids that are not conserved.

In some embodiments, a variant of a polypeptide comprises a heterologous polypeptide portion. The heterologous portion often has a sequence that is not present in or homologous to the original polypeptide. A heterologous portion may be, e.g., between 5 and about 5,000 amino acids long, or longer. Often it is between 5 and about 1,000 amino acids long. In some embodiments, a heterologous portion comprises a sequence that is found in a different polypeptide, e.g., a functional domain. In some embodiments, a heterologous portion comprises a sequence useful for purifying, expressing, solubilizing, and/or detecting the polypeptide. In some embodiments, a heterologous portion comprises a polypeptide “tag”, e.g., an affinity tag or epitope tag. For example, the tag can be an affinity tag (e.g., HA, TAP, Myc, His, Flag, GST), fluorescent or luminescent protein (e.g., EGFP, ECFP, EYFP, Cerulean, DsRed, mCherry), solubility-enhancing tag (e.g., a SUMO tag, NUS A tag, SNUT tag, or a monomeric mutant of the Ocr protein of bacteriophage T7). See, e.g., Esposito D and Chatterjee D K. Curr Opin Biotechnol.; 17(4):353-8 (2006). In some embodiments, a tag can serve multiple functions. A tag is often relatively small, e.g., ranging from a few amino acids up to about 100 amino acids long. In some embodiments a tag is more than 100 amino acids long, e.g., up to about 500 amino acids long, or more. In some embodiments, a polypeptide has a tag located at the N- or C-terminus, e.g., as an N- or C-terminal fusion. The polypeptide could comprise multiple tags. In some embodiments, a His tag and a NUS tag are present, e.g., at the N-terminus. In some embodiments, a tag is cleavable, so that it can be removed from the polypeptide, e.g., by a protease. In some embodiments, this is achieved by including a sequence encoding a protease cleavage site between the sequence encoding the portion homologous to the original polypeptide and the tag. Exemplary proteases include, e.g., thrombin, TEV protease, Factor Xa, PreScission protease, etc. In some embodiments, a “self-cleaving” tag is used. See, e.g., PCT/US05/05763. Sequences encoding a tag can be located 5′ or 3′ with respect to a polynucleotide encoding the polypeptide (or both). In some embodiments a tag or other heterologous sequence is separated from the rest of the polypeptide by a polypeptide linker. For example, a linker can be a short polypeptide (e.g., 15-25 amino acids). Often a linker is composed of small amino acid residues such as serine, glycine, and/or alanine. A heterologous domain could comprise a transmembrane domain, a secretion signal domain, etc.

In certain embodiments of the invention a fragment or variant, optionally excluding a heterologous portion, if present, possesses sufficient structural similarity to the original polypeptide so that when its 3-dimensional structure (either actual or predicted structure) is superimposed on the structure of the original polypeptide, the volume of overlap is at least 70%, preferably at least 80%, more preferably at least 90% of the total volume of the structure of the original polypeptide. A partial or complete 3-dimensional structure of the fragment or variant may be determined by crystallizing the protein, which can be done using standard methods. Alternately, an NMR solution structure can be generated, also using standard methods. A modeling program such as MODELER (Sali, A. and Blundell, T L, J. Mol. Biol., 234, 779-815, 1993), or any other modeling program, can be used to generate a predicted structure. If a structure or predicted structure of a related polypeptide is available, the model can be based on that structure. The PROSPECT-PSPP suite of programs can be used (Guo, J T, et al., Nucleic Acids Res. 32 (Web Server issue):W522-5, Jul. 1, 2004). Where embodiments of the invention relate to variants of a polypeptide, it will be understood that polynucleotides encoding the variant are provided.

The term “vector” is used herein to refer to a nucleic acid or a virus or portion thereof (e.g., a viral capsid or genome) capable of mediating entry of, e.g., transferring, transporting, etc., a nucleic acid molecule into a cell. Where the vector is a nucleic acid, the nucleic acid molecule to be transferred is generally linked to, e.g., inserted into, the vector nucleic acid molecule. A nucleic acid vector may include sequences that direct autonomous replication (e.g., an origin of replication), or may include sequences sufficient to allow integration of part or all of the nucleic acid into host cell DNA. Useful nucleic acid vectors include, for example, DNA or RNA plasmids, cosmids, and naturally occurring or modified viral genomes or portions thereof or nucleic acids (DNA or RNA) that can be packaged into viral) capsids. Plasmid vectors typically include an origin of replication and one or more selectable markers. Plasmids may include part or all of a viral genome (e.g., a viral promoter, enhancer, processing or packaging signals, etc.). Viruses or portions thereof that can be used to introduce nucleic acid molecules into cells are referred to as viral vectors. Useful viral vectors include adenoviruses, adeno-associated viruses, retroviruses, lentiviruses, vaccinia virus and other poxviruses, herpesviruses (e.g., herpes simplex virus), and others. Viral vectors may or may not contain sufficient viral genetic information for production of infectious virus when introduced into host cells, i.e., viral vectors may be replication-defective, and such replication-defective viral vectors may be preferable for therapeutic use. Where sufficient information is lacking it may, but need not be, supplied by a host cell or by another vector introduced into the cell. The nucleic acid to be transferred may be incorporated into a naturally occurring or modified viral genome or a portion thereof or may be present within the virus or viral capsid as a separate nucleic acid molecule. It will be appreciated that certain plasmid vectors that include part or all of a viral genome, typically including viral genetic information sufficient to direct transcription of a nucleic acid that can be packaged into a viral capsid and/or sufficient to give rise to a nucleic acid that can be integrated into the host cell genome and/or to give rise to infectious virus, are also sometimes referred to in the art as viral vectors. Vectors may contain one or more nucleic acids encoding a marker suitable for use in the identifying and/or selecting cells that have or have not been transformed or transfected with the vector. Markers include, for example, proteins that increase or decrease either resistance or sensitivity to antibiotics (e.g., an antibiotic-resistance gene encoding a protein that confers resistance to an antibiotic such as puromycin, hygromycin or blasticidin) or other compounds, enzymes whose activities are detectable by assays known in the art (e.g., beta.-galactosidase or alkaline phosphatase), and proteins or RNAs that detectably affect the phenotype of transformed or transfected cells (e.g., fluorescent proteins). Expression vectors are vectors that include regulatory sequence(s), e.g., expression control sequences such as a promoter, sufficient to direct transcription of an operably linked nucleic acid. Regulatory sequences may also include enhancer sequences or upstream activator sequences. Vectors may optionally include 5′ leader or signal sequences. Vectors may optionally include cleavage and/or polyadenylations signals and/or a 3′ untranslated regions. Vectors often include one or more appropriately positioned sites for restriction enzymes, to facilitate introduction into the vector of the nucleic acid to be expressed. An expression vector comprises sufficient cis-acting elements for expression; other elements required or helpful for expression can be supplied by the host cell or in vitro expression system.

Various techniques may be employed for introducing nucleic acid molecules into cells. Such techniques include chemical-facilitated transfection using compounds such as calcium phosphate, cationic lipids, cationic polymers, liposome-mediated transfection, non-chemical methods such as electroporation, particle bombardment, or microinjection, and infection with a virus that contains the nucleic acid molecule of interest (sometimes termed “transduction”). Markers can be used for the identification and/or selection of cells that have taken up the vector and, typically, express the nucleic acid. Cells can be cultured in appropriate media to select such cells and, optionally, establish a stable cell line.

Before the present invention is described in greater detail, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges and are also encompassed within the invention, subject to any specifically excluded limit in the stated range. Where the stated range includes one or both of the limits, ranges excluding either or both of those included limits are also included in the invention.

Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number which, in the context in which it is presented, provides the substantial equivalent of the specifically recited number.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, representative illustrative methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present invention is not entitled to antedate such publication by virtue of prior invention. Further, the dates of publication provided may be different from the actual publication dates which may need to be independently confirmed.

It is noted that, as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

The present disclosure provides novel compositions and methods for maturing mammalian cells including human cells either in vivo or in vitro and methods of use thereof. In some aspects, the invention provides novel methods of providing mature cells when said cells are derived from pluripotent stem cells and despite being capable of differentiation or despite being fully differentiated, they are nonetheless immature in that they express markers of embryonic (pre-fetal) cells before the EFT or are, in any event, not expressing a pattern of gene expression similar to their normal adult counterparts as evidenced by them displaying a plurality of embryonic (pre-fetal) markers and not expressing a plurality of fetal and adult markers, said markers being previously disclosed (see PCT International Patent Application No. PCT/US2020/025512, titled “Induced tissue regeneration using extracellular vesicles,” filed Mar. 27, 2020; PCT International Patent Application No. PCT/US14/40601, titled “Compositions and Methods for Induced Tissue Regeneration in Mammalian Species,” filed Jun. 3, 2014; and PCT International Patent Application No. PCT/US2017/036452, titled “Improved Methods for Detecting and Modulating the Embryonic-Fetal Transition in Mammalian Cells,” filed Jun. 7, 2017, U.S. Provisional Application No. 63/274,734, titled “Methods and Compositions Used to Modify Chromatin Architecture to Regulate Phenotype in Aging and Cancer,” filed Nov. 2, 2021; and U.S. Provisional Application No. 63/256,286, titled “Methods for Modulating the Regenerative Phenotype in Mammalian Cells,” filed Oct. 15, 2021, contents of each of which are incorporated herein by reference). Said compositions and methods for enhancing the maturation of cells disclosed herein has utility in generating cells that more fully represent their normal adult counterparts useful in screening drugs intended for potential use in adult mammals such as humans for efficacy or toxicity.

The applicants teach that primitive animals that display the potential for profound TR such as the regeneration of amputated limbs in axolotls, the regeneration of skin in MRL or the African Spiny Mouse, or the regeneration of whole body segments in planaria, do so by simply recapitulating normal embryonic development of the respective tissues. Furthermore, the applicants teach that the cause of inability to regenerate damaged tissue in TR-resistant animals, such as the majority of adult mammals such as most murine species and humans is that certain embryonic gene transcription is altered at or around the time of the EFT (exact timing varying with tissue-type). The applicants further teach that the restoration of certain of these embryo-specific patterns of gene expression altered in the EFT in TR-resistant animals can induce competency for regeneration in any tissue, including responsiveness to organizing center factors, leading to complex tissue regeneration and a concomitant reduction in scar formation. Furthermore, the applicants teach that the maturation (as opposed to differentiation) of mammalian somatic cells that occurs during development in utero in the case of placental mammals, is regulated in part by endocrine signaling. The development of the pituitary and thyroid gland during embryogenesis provides for the expression of thyroid stimulating hormone, proopiomelanocortin (POMC), thyroid hormone (both T4 and T3) as well as glucocorticoid hormones such as POMC-derived corticotropin and adrenal cortisol. The applicants teach that the evolution or tetrapods from previously aquatic species led to amniotes as opposed to anamniotes, and in order to regulate said endocrine signaling, novel endocrine pathways evolved associated with metamorphosis. In the case of viviparous animals such as mammals, which is the subject of the present invention, the adult-like endocrine environment of the mother needed to be partitioned away from the developing conceptus to allow the previous metamorphic pathways to be functional. As a result, the applicants teach that the normal endocrine mileau of the developing mammalian fetus and subsequent infant is the source of said endocrine maturation signaling and may be utilized to induce the maturation of PSC-derived cells. Lastly, the applicants teach that said cell maturation is a previously-unidentified tumor suppression mechanism. While repressing the natural regenerative potential of mammalian somatic cells and tissue to regenerate following injury or disease, it nonetheless provides a barrier to the development of cancer cells by slowing cell division. As a result, novel agents and associated methods of inducing iTM also have the effect of iCM and resulting tumor suppression in mammalian species. Said methods facilitate iTM and iCM in mammalian species in vitro and in vivo, particularly in the species Homo sapiens. Applications of said iTM and iCM compositions and methods include the maturation of PSC-derived cells in vitro or in vivo (for example following transplantation and engraftment of PSC-derived cells), and for the suppression of cancer.

Genes whose expression in fetal and adult animals inhibit TR are herein designated “TR inhibitors” or sometimes referred to as “iTR Inhibitors”, and genes whose lack of expression in fetal and adult cells but whose expression in embryonic phases of development facilitate TR are herein designated “TR activators.” Collectively, TR inhibitor genes and TR activator genes are herein designated iTR genes. Molecules that alter the levels of TR activators and TR inhibitors in a manner leading to TR are herein designated “iTR factors” and are previously disclosed (see PCT International Patent Application No. PCT/US14/40601, titled “Compositions and Methods for Induced Tissue Regeneration in Mammalian Species,” filed Jun. 3, 2014; and PCT International Patent Application No. PCT/US2017/036452, titled “Improved Methods for Detecting and Modulating the Embryonic-Fetal Transition in Mammalian Cells,” filed Jun. 7, 2017, U.S. Provisional Application No. 63/274,734, titled “Methods and Compositions Used to Modify Chromatin Architecture to Regulate Phenotype in Aging and Cancer,” filed Nov. 2, 2021; and U.S. Provisional Application No. 63/256,286, titled “Methods for Modulating the Regenerative Phenotype in Mammalian Cells,” filed Oct. 15, 2021, contents of each of which are incorporated herein by reference). iTR genes and, the protein products of iTR genes, are often conserved in animals ranging from sea anemones to mammals. The gene-encoded protein sequences, and sequences of nucleic acids (e.g., mRNA) encoding genes referred to herein, including those from a number of different non-human animal species are known in the art and can be found, e.g., in publicly available databases such as those available at the National Center for Biotechnology Information (NCBI) (www.ncbi.nih.gov).

By way of nonlimiting example, the TR inhibitory gene COX7A1 is observed to be expressed broadly in diverse fetal and adult somatic cell types but rarely expressed in clonal EP cell lines and often not expressed in sarcoma, carcinoma, and adenocarcinoma cell lines. The exogenous induction of expression of the TR inhibitory genes disclosed herein and previously-disclosed (see PCT International Patent Application No. PCT/US14/40601, titled “Compositions and Methods for Induced Tissue Regeneration in Mammalian Species,” filed Jun. 3, 2014; and PCT International Patent Application No. PCT/US2017/036452, titled “Improved Methods for Detecting and Modulating the Embryonic-Fetal Transition in Mammalian Cells,” filed Jun. 7, 2017, U.S. Provisional Application No. 63/274,734, titled “Methods and Compositions Used to Modify Chromatin Architecture to Regulate Phenotype in Aging and Cancer,” filed Nov. 2, 2021; and U.S. Provisional Application No. 63/256,286, titled “Methods for Modulating the Regenerative Phenotype in Mammalian Cells,” filed Oct. 15, 2021, contents of each of which are incorporated herein by reference) in such tumors lacking expression would therefore have a therapeutic effect by slowing the growth of the cancer cells and are designated herein as segmental iTM or segmental iCM genes.

In another embodiment, the present invention provides a means of globally inducing iTM and iCM in non-cancerous mammalian, including human, somatic cells and cancer cells with an embryonic (pre-fetal) pattern of gene expression, said compositions and methods comprising exposing said non-cancerous or cancer cells with molecules that mimic the endocrine environment that induces cell maturation at or around the time of the EFT. An example of said endocrine factors are thyroid hormones (TH) T3 and T4, cortisol or synthetic analogues of cortisol such as dexamethasone, FGF7, growth hormone (GH), combinations thereof, including without limitation, the combined application of TH and dexamethasone. Said effectors of global iTM or iCM may also be combined when useful with segmental iTM or iCM factors also described herein.

In general, an iTM or iCM factor can be, e.g., a small molecule, nucleic acid, oligonucleotide, polypeptide, peptide, lipid, carbohydrate, etc. In some embodiments of the invention, iTM or iCM factors inhibit by decreasing the amount of TR activator RNA produced by cells and/or by decreasing the level of activity of TR activator genes. In the case of targeting TR activators, factors are identified and used in research and therapy that reduce the levels of the product of the TR activator gene such as by RNAi. Said TR activator gene can be any one or combination of the products of the AFF3, CBCAQH03 5, DLX1, DRD11P, F2RL2, FOXD1, LOC728755, LOC791120, MN1, OXTR, PCDHB2, PCDHB17, RAB3IP, SIX1, and WSB1 (see PCT/US14/40601, filed Jun. 3, 2014 and titled “Compositions and Methods for Induced Tissue Regeneration in Mammalian Species”); the genes: ADGRV1, AFF3, ALDH5A1, ALX1, AMH, B4GALNT4, C14orf39, CHKB-CPT1B, CPT1B, DOC2GP, DPY19L2, DSG2, FAM157A, FAM157B, FOXD4L4, FSIP2, GDF1, GRIN3B, H2BFXP, L3MBTL1, LIN28B, LINC00649, LINC01021, LINC01116, NAALAD2, PAQR6, members of the alpha clustered protocadherin locus A2-11, members of the beta clustered protocadherin locus B2-17, PCDHGB4, PCDHGB6, PLPPR3, PRR5L, RGPD1, SLCO1A2, TSPAN11, TUBB2B, ZCCHC18, ZNF497, and ZNF853 (see PCT/US2017/036452, filed Jun. 7, 2017 and titled “Improved Methods for Detecting and Modulating the Embryonic-Fetal Transition in Mammalian Species,”); and the gene LMNB1 (see U.S. Provisional Application No. 63/274,734, titled “Methods and Compositions Used to Modify Chromatin Architecture to Regulate Phenotype in Aging and Cancer,” filed Nov. 2, 2021, contents of which are incorporated herein by reference); and the genes: AC108142.1, AGA, AQP7P1, AQP7P3, BAHD1, BBOX1, C11orf35 (LMNTD2), CASC9, CBX2, CCDC144NL, CHRM3, CPAMD8, FAR2P1, FAR2P2, FAR2P3, FIRRE, IGF2BP1, LINC00649, LINC02315, LOC644919, MED15P9, PCAT7, PKP3, POTEE, POTEF, PURPL, RGPD2, WDR72, or WRN (see U.S. Provisional Application No. 63/256,286, titled “Methods for Modulating the Regenerative Phenotype in Mammalian Cells,” filed Oct. 15, 2021, contents of each of which are incorporated herein by reference).

To accomplish maturation (e.g., segmental iTM or segmental iCM), said TR activator gene can be any one or combination of TR activator genes listed herein. The amount of TR activator gene RNA can be decreased by inhibiting synthesis of TR activator RNA synthesis by cells (also referred to as “inhibiting TR activator gene expression”), e.g., by reducing the amount of mRNA encoding TR activator genes or by reducing translation of mRNA encoding TR activator genes. Said factor can be by way of nonlimiting example, RNAi targeting a sequence within the genes for TR activator described herein.

In some embodiments, TR activator gene expression is inhibited by RNA interference (RNAi). As known in the art, RNAi is a process in which the presence in a cell of double-stranded RNA that has sequence correspondence to a gene leads to sequence-specific inhibition of the expression of the gene, typically as a result of cleavage or translational repression of the mRNA transcribed from the gene. Compounds useful for causing inhibition of expression by RNAi (“RNAi agents”) include short interfering RNAs (siRNAs), short hairpin RNAs (shRNAs), microRNAs (miRNAs), and miRNA-like molecules.

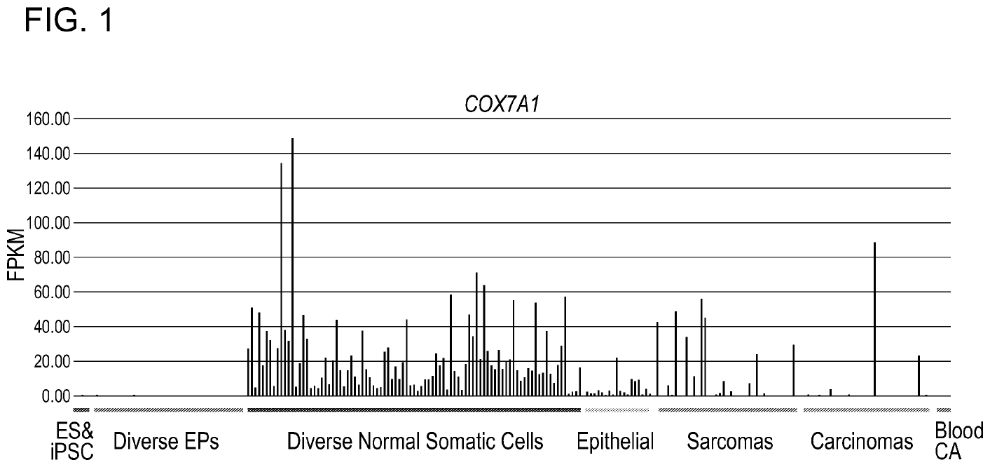

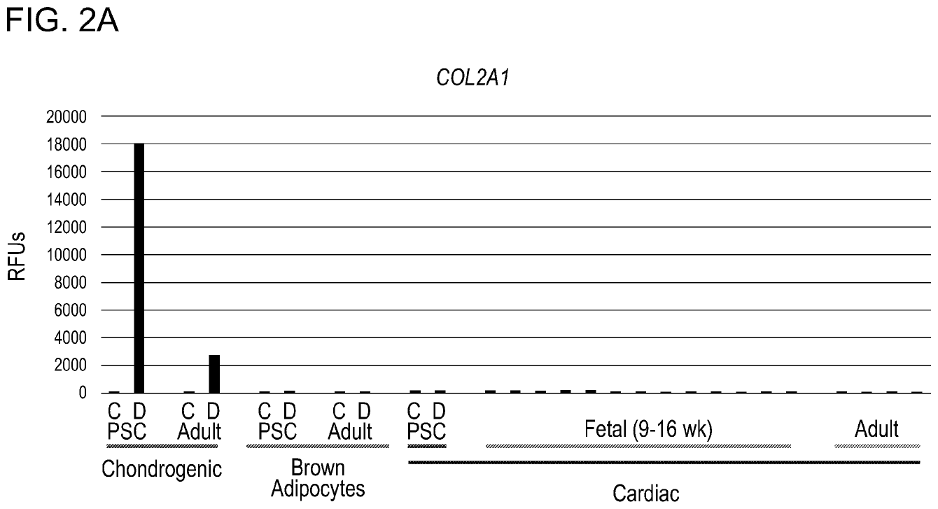

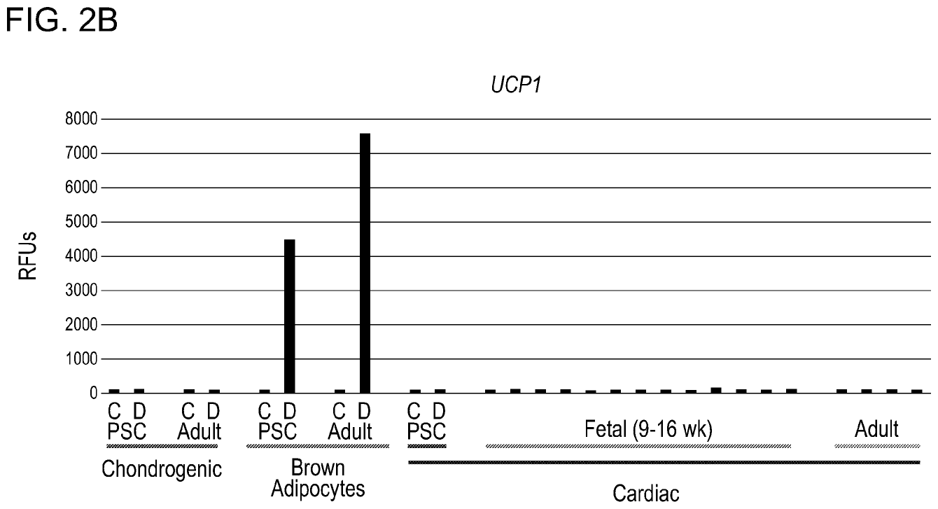

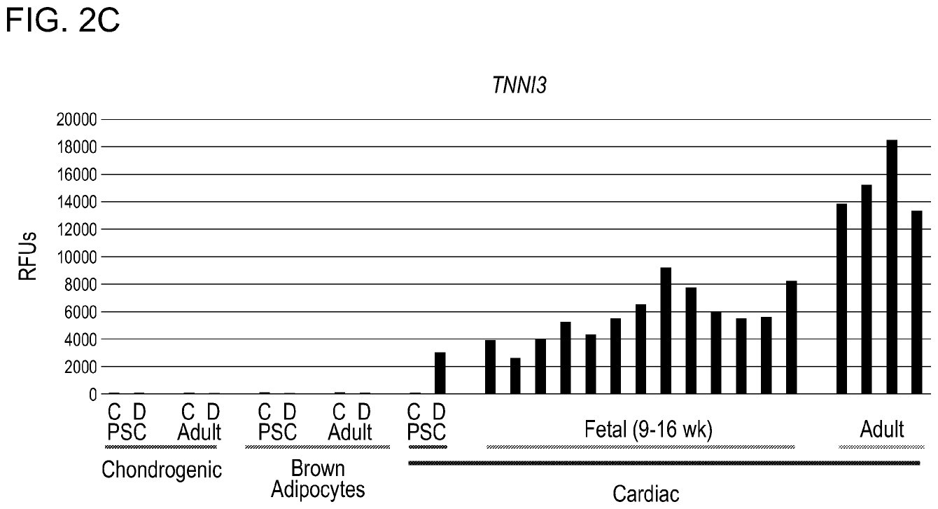

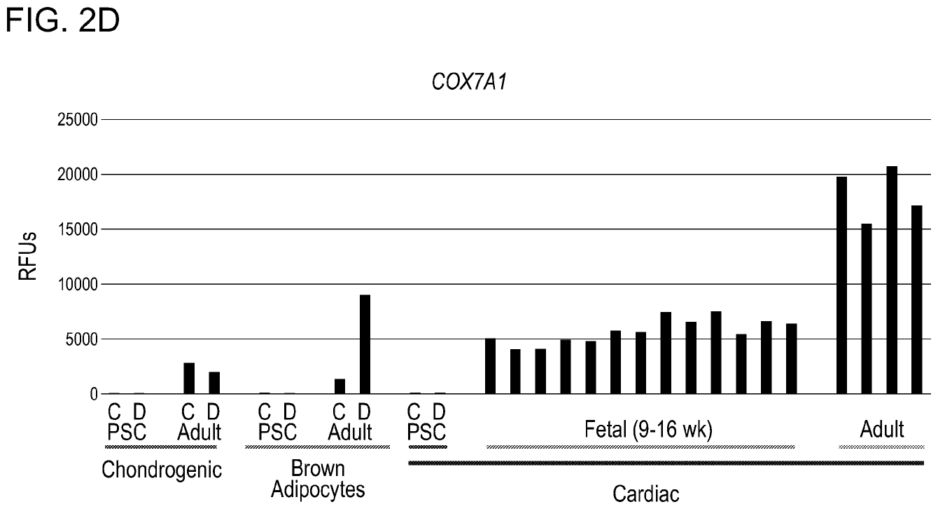

Model systems for use in uncovering the molecular mechanisms underlying the transition in mammals from a pre-EFT regenerative phenotype to a post-EFT nonregenerative phenotype were made possible through the use of previously-disclosed pluripotent stem cell-derived clonal embryonic progenitor cell lines that could in turn be compared to their adult counterparts to uncover molecular alterations during the transition from pre-EFT to post-EFT that are common among diverse somatic cell types. Therefore, in addition to the methods described below, methods that find use in the production and use of cells with an embryonic pattern of gene expression corresponding with scarless regenerative potential can be found in the following: PCT application Ser. No. PCT/US2006/013519 filed on Apr. 11, 2006 and titled “Novel Uses of Cells With Prenatal Patterns of Gene Expression”; U.S. patent application Ser. No. 11/604,047 filed on Nov. 21, 2006 and titled “Methods to Accelerate the Isolation of Novel Cell Strains from Pluripotent Stem Cells and Cells Obtained Thereby”; and U.S. patent application Ser. No. 12/504,630 filed on Jul. 16, 2009 and titled “Methods to Accelerate the Isolation of Novel Cell Strains from Pluripotent Stem Cells and Cells Obtained Thereby”, (See, e.g. U.S. provisional patent application No. 61/831,421, filed Jun. 5, 2013, PCT patent application PCT/US2014/040601, filed Jun. 3, 2014 and U.S. patent application Ser. No. 14/896,664, filed on Dec. 7, 2015, the disclosures of which are incorporated by reference in their entirety), each of which is incorporated by reference herein in its entirety. Genes that were discovered to be profoundly differentially expressed in diverse clonal embryonic progenitor cells compared to their adult counterparts were then analyzed for their potential role as iTR activators (or iCM and iTM inhibitors) when the genes were expressed in a wide array of somatic cell types in the pre-EFT stages but not the later fetal or adult stages of development, and iTR inhibitors (or iCM and iTM activators) when said genes were expressed in diverse somatic cell types only after the EFT, that is, in the fetal or adult stages of development. An example of said iTR inhibitor (or iCM and iTM activator) gene previously disclosed (see PCT International Patent Application No. PCT/US14/40601, titled “Compositions and Methods for Induced Tissue Regeneration in Mammalian Species,” filed Jun. 3, 2014; and PCT International Patent Application No. PCT/US2017/036452, titled “Improved Methods for Detecting and Modulating the Embryonic-Fetal Transition in Mammalian Cells,” filed Jun. 7, 2017, contents of each of which are incorporated herein by reference) is COX7A1 which encodes a protein that increases oxidative phosphorylation and its expression in PSCs, diverse clonal embryonic progenitor cell lines, diverse fetal and adult cell types, and diverse cancer cell lines is shown in FIG. 1. COX7A1 is expressed in fetal and adult diverse somatic cell types from all three germ layers, but not in the embryonic (pre-fetal) stages of development, despite the fact that said embryonic (pre-fetal) cells may be fully differentiated in the sense that they express markers of neurons, skeletal or cardiac muscle cells, brown fat, cartilage and so on. Therefore, maturation through the EFT and the consequent loss of regenerative potential is distinct from differentiation. A tissue may have profound regenerative potential despite the cells in that tissue being fully differentiated. The tissue will regenerate if the cells in said tissue have not matured past the EFT, which, in the case of humans is approximately eight weeks of gestation, however, the exact timing of maturation various with the tissue type and the timing of the assembly of Lamin A in the nuclear lamina. Surprisingly, cells derived in vitro from PSCs may terminally differentiate, but generally do not mature. By way of nonlimiting examples, pluripotent stem cell-derived clonal embryonic progenitor cell lines may be differentiated into cartilage (Sternberg H. et al, Seven diverse human embryonic stei cell-derived chondrogenic clonal embryonic progenitor cell lines display site-specific cell fates. Regen Med. 2013 March; 8(2):125-44. doi: 10.2217/rme,12.117. Epub 2012 Dec. 18. PMID: 23249126), brown adipocytes (West M D. et al, Clonal derivation of white and brown adipocyte progenitor cell lines from human pluripotent stem cells. Stein Cell Res Ther. 2019 Jan. 8; 10(1):7. doi: 10.1186/s13287-018-1087-7. PMID: 30616682; PMCID: PMC6323697), or cardiac cells (see PCT International Application No. PCT/US2020/012640, titled “Compositions and Methods for Detecting Cardiotoxicity,” filed Jan. 7, 2020, contents of each of which are incorporated herein by reference) while nevertheless not fully maturing into fetal or adult-like gene expression as evidenced, by way of nonlimiting example, a lack of COX7A1 expression (FIGS. 2A-2D). In the case of applications wherein PSC-derived cells are desired for applications in vitro such as in drug screening of cardiomyocytes, adult-like cells can be obtained by segmental iTM wherein COX7A1 is introduced exogenously to produce cardiomyocytes with adult-like metabolism (see PCT International Application No. PCT/US2020/012640, titled “Compositions and Methods for Detecting Cardiotoxicity,” filed Jan. 7, 2020, contents of each of which are incorporated herein by reference).

### Methods

### Global iTM and iCM Factors

Global iTM and iCM factors, e.g., endocrine factors, are those molecules that advance the maturation of diverse mammalian somatic cells from an embryonic (pre-fetal) pattern of gene expression to a pattern of gene expression corresponding to later fetal or adult cells. Global iTM and iCM factors (e.g., endocrine factors) differ from segmental iTM and iCM factors in that global iTM and iCM factors (e.g., endocrine factors) alter a plurality of genes toward that of a later fetal or adult-like state.

Global iTM and iCM factors (e.g., endocrine factors) include the following concentrations. According to some embodiments, the endocrine factor T3 is used at a concentration of about 0.2 to about 20 nM, preferably 2 nM of T3. According to some embodiments, T3 is used at a concentration of at least 0.2 nM, at least 0.5 nM, at least 0.7 nM, at least 1 nM, at least 1.2 nM, at least 1.5 nM, at least 1.7 nM, at least 2.0 nM, at least 2.2 nM, at least 2.5 nM, at least 3.0 nM, at least 3.2 nM, at least 3.5 nM, at least 3.7 nM, at least 4.0 nM, at least 4.2 nM, at least 4.5 nM, at least 5.0 nM, at least 5.2 nM, at least 5.5. nM, at least 5.7 nM, at least 6.0 nM, at least 6.2 nM, at least 6.5 nM, at least 6.7 nM, at least 7.0 nM, at least 7.2 nM, at least 7.5 nM, at least 7.7 nM, at least 8.0 nM, at least 8.2 nM, at least 8.5 nM, at least 8.7 nM, at least 9.0 nM, at least 9.2 nM, at least 9.5 nM, at least 9.7 nM, at least 10.0 nM, at last 10.5 nM, at least 11.0 nM, at least 11.5 nM, at least 12.0 nM, at least 12.5 nM, at least 13.0 nM, at least 13.5 nM, at least 14.0 nM, at least 14.5 nm, at least 15.0 nM, at least 15.5 nM, at least 16.0 nM, at least 16.5 nM, at least 17.0 nM, at least 17.5 nM, at least 18.0 nM, at least 18.5 nM, 19.0 nM, at least 19.5 nM, or at least 20.0 nM.

According to some embodiments, T4 is used at a concentration of about 1.0 to about 100 ng/ml, preferably 10 ng/ml of T4. According to some embodiments, T4 is used at a concentration of at least 1.0 ng/mL, at least 2.0 ng/mL, at least 3.0 ng/mL, at least 4.0 ng/mL, at least 4.0 ng/mL, at least 5.0 ng/mL, at least 6.0 ng/mL, at least 7.0 ng/mL, at least 8.0 ng/mL, a least 9.0 ng/mL, at least 10.0 ng/mL, at least 15.0 ng/mL, at least 16.0 ng/mL, at least 17.0 ng/mL, at least 18.0 ng/mL, at least 19.0 ng/mL, at least 20.0 ng/mL, at least 25.0 ng/mL, at least 30.0 ng/mL, at least 35.0 ng/mL, at least 40.0 ng/mL, at least 45.0 ng/mL, at least 50.0 ng/mL, at least 55.0 ng/mL, at least 60.0 ng/mL, at least 65.0 ng/mL, at least 70.0 ng/mL, at least 75.0 ng/mL, at least 80.0 ng/mL, at least 85.0 ng/mL, at least 90.0 ng/mL, at least 95.0 ng/mL, or at least 100.0 ng/mL.

According to some embodiments, dexamethasone is used at a concentration of about 0.01 to about 1.0 uM, preferably 0.1 uM. In some embodiments, dexamethasone is used at a concentration of at least 0.01 uM, at least 0.02 uM, at least 0.03 uM, at least 0.04 uM, at least 0.05 uM, at least 0.06 uM, at least 0.07 uM, at least 0.08 uM, at least 0.09 uM, at least 0.1 uM, at last 0.2 uM, at least 0.3 uM, at least 0.4 uM, at least 0.5 uM, at last 0.6 uM, at least 0.7 uM, at least 0.8 uM, at least 0.9 uM, or at least 1.0 uM.

According to some embodiments, cortisol is used at a concentration of about 1.0 to about 100 nM, preferably 10 nM. According to come embodiments, cortisol is used at a concentration of at least is used at a concentration of at least 1.0 nM, at least 2.0 nM, at least 3.0 nM, at least 4.0 nM, at least 4.0 nM, at least 5.0 nM, at least 6.0 nM, at least 7.0 nM, at least 8.0 nM, a least 9.0 nM, at least 10.0 nM, at least 15.0 nM, at least 16.0 nM, at least 17.0 nM, at least 18.0 nM, at least 19.0 nM, at least 20.0 nM, at least 25.0 nM, at least 30.0 nM, at least 35.0 nM, at least 40.0 nM, at least 45.0 nM, at least 50.0 nM, at least 55.0 nM, at least 60.0 nM, at least 65.0 nM, at least 70.0 nM, at least 75.0 nM, at least 80.0 nM, at least 85.0 nM, at least 90.0 nM, at least 95.0 nM, or at least 100.0 nM.

According to some embodiments, growth hormone (GH) is used at a concentration of about 0.1 to about 10 ng/ml, preferably 1.0 ng/ml of growth hormone (GH). According to some embodiments, growth hormone is used at a concentration of at least 0.01 ng/ml, at least 0.02 ng/ml, at least 0.03 ng/ml, at least 0.04 ng/ml, at least 0.05 ng/ml, at least 0.06 ng/ml, at least 0.07 ng/ml, at least 0.08 ng/ml, at least 0.09 ng/ml, at least 0.1 ng/ml, at last 0.2 ng/ml, at least 0.3 ng/ml, at least 0.4 ng/ml, at least 0.5 ng/ml, at last 0.6 ng/ml, at least 0.7 ng/ml, at least 0.8 ng/ml, at least 0.9 ng/ml, or at least 1.0 ng/ml.

According to some embodiments, insulin-like growth factor-2 (IGF2) is used at a concentration of about 1.0-100 ng/ml, preferably 10 ng/ml of insulin-like growth factor-2 (IGF2). According to come embodiments, insulin-like growth factor-2 (IGF2) is used at a concentration of at least is used at a concentration of at least 1.0 ng/ml, at least 2.0 ng/mL, at least 3.0 ng/ml, at least 4.0 ng/ml, at least 4.0 ng/ml, at least 5.0 ng/ml, at least 6.0 ng/ml, at least 7.0 ng/ml, at least 8.0 ng/ml, a least 9.0 ng/ml, at least 10.0 ng/ml, at least 15.0 ng/ml, at least 16.0 ng/ml, at least 17.0 ng/ml, at least 18.0 ng/ml, at least 19.0 ng/ml, at least 20.0 ng/ml, at least 25.0 ng/ml, at least 30.0 ng/ml, at least 35.0 ng/ml, at least 40.0 ng/ml, at least 45.0 ng/ml, at least 50.0 ng/ml, at least 55.0 ng/ml, at least 60.0 ng/ml, at least 65.0 ng/ml, at least 70.0 ng/ml, at least 75.0 ng/ml, at least 80.0 ng/ml, at least 85.0 ng/ml, at least 90.0 ng/ml, at least 95.0 ng/ml, or at least 100.0 ng/ml.

According to some embodiments, fibroblast growth factor-7 (FGF7) is used at a concentration of about 1.0 to about 100 ng/ml, preferably 10 ng/ml of fibroblast growth factor-7 (FGF7). According to come embodiments, FGF7 is used at a concentration of at least is used at a concentration of at least 1.0 ng/ml, at least 2.0 ng/mL, at least 3.0 ng/ml, at least 4.0 ng/ml, at least 4.0 ng/ml, at least 5.0 ng/ml, at least 6.0 ng/ml, at least 7.0 ng/ml, at least 8.0 ng/ml, a least 9.0 ng/ml, at least 10.0 ng/ml, at least 15.0 ng/ml, at least 16.0 ng/ml, at least 17.0 ng/ml, at least 18.0 ng/ml, at least 19.0 ng/ml, at least 20.0 ng/ml, at least 25.0 ng/ml, at least 30.0 ng/ml, at least 35.0 ng/ml, at least 40.0 ng/ml, at least 45.0 ng/ml, at least 50.0 ng/ml, at least 55.0 ng/ml, at least 60.0 ng/ml, at least 65.0 ng/ml, at least 70.0 ng/ml, at least 75.0 ng/ml, at least 80.0 ng/ml, at least 85.0 ng/ml, at least 90.0 ng/ml, at least 95.0 ng/ml, or at least 100.0 ng/ml.

According to some embodiments, Tri(1,3-dichloropropyl) phosphate (TDCPP) is used at a concentration of about 5.0 to about 500 mg/kg/day, preferably 50 ng/kg/day in vivo or 50 μg/ml in vitro of Tri(1,3-dichloropropyl) phosphate (TDCPP). According to some embodiments, TDCCP is used in vivo at a concentration of at least 5.0 mg/kg/day, at least 10.0 mg/kg/day, at least 15.0 mg/kg/day, at least 20.0 mg/kg/day, at least 25.0 mg/kg/day, at least 30.0 mg/kg/day, at least 35.0 mg/kg/day, at least 40.0 mg/kg/day, at least 45.0 mg/kg/day, at least 50.0 mg/kg/day, at least 60.0 mg/kg/day, at least 70.0 mg/kg/day, at least 80.0 mg/kg/day, at least 90.0 mg/kg/day, at least 100.0 mg/kg/day, at least 120.0 mg/kg/day, at least 140.0 mg/kg/day, at least 160.0 mg/kg/day, at least 180.0 mg/kg/day, at least 200.0 mg/kg/day, at least 220.0 mg/kg/day, at least 240.0 mg/kg/day, at least 260.0 mg/kg/day, at least 280.0 mg/kg/day, at least 300.0 mg/kg/day, at least 320.0 mg/kg/day, at least 340.0 mg/kg/day, at least 360.0 mg/kg/day, at least 380.0 mg/kg/day, at least 400.0 mg/kg/day, at least 420.0 mg/kg/day, at least 440.0 mg/kg/day, at least 460.0 mg/kg/day, at least 480.0 mg/kg/day, or at least 500.0 mg/kg/day. According to come embodiments, TDCCP is used in vitro at a concentration of at least 5.0 ug/ml, at least 10.0 ug/ml, at least 20.0 ug/ml, at least 30.0 ug/ml, at least 40.0 ug/ml, at least 50.0 ug/ml, at least 60.0 ug/ml, at least 70.0 ug/ml, at least 80.0 ug/ml, at least 90.0 ug/ml, or at least 100.0 ug/ml.

According to some embodiments, bis(1,3-dichloro-2-propyl) phosphate (IBDCPP) is used at a concentration of about 5.0 to about 500 mg/kg/day, preferably 50 ng/kg/day in vivo or 50 μg/ml in vitro of bis(1,3-dichloro-2-propyl) phosphate (BDCPP). According to some embodiments. BDCPP is used in vivo at a concentration of at least 5.0 mg/kg/day, at least 10.0 mg/kg/day, at least 15.0 mg/kg/day, at least 20.0 mg/kg/day, at least 25.0 mg/kg/day, at least 30.0 mg/kg/day, at least 35.0 mg/kg/day, at least 40.0 mg/kg/day, at least 45.0 mg/kg/day, at least 50.0 mg/kg/day, at least 60.0 mg/kg/day, at least 70.0 mg/kg/day, at least 80.0 mg/kg/day, at least 90.0 mg/kg/day, at least 100.0 mg/kg/day, at least 120.0 mg/kg/day, at least 140.0 mg/kg/day, at least 160.0 mg/kg/day, at least 180.0 mg/kg/day, at least 200.0 mg/kg/day, at least 220.0 mg/kg/day, at least 240.0 mg/kg/day, at least 260.0 mg/kg/day, at least 280.0 mg/kg/day, at least 300.0 mg/kg/day, at least 320.0 mg/kg/day, at least 340.0 mg/kg/day, at least 360.0 mg/kg/day, at least 380.0 mg/kg/day, at least 400.0 mg/kg/day, at least 420.0 mg/kg/day, at least 440.0 mg/kg/day, at least 460.0 mg/kg/day, at least 480.0 mg/kg/day, or at least 500.0 mg/kg/day. According to come embodiments, BDCPP is used in vitro at a concentration of at least 5.0 ug/ml, at least 10.0 ug/ml, at least 20.0 ug/ml, at least 30.0 ug/ml, at least 40.0 ug/ml, at least 50.0 ug/ml, at least 60.0 ug/ml, at least 70.0 ug/ml, at least 80.0 ug/ml, at least 90.0 ug/ml, or at least 100.0 ug/ml.

Since T3 hormone is inactivated in some cells by Deiodinase 3 (DI03), cells and tissues that express the DI03 gene such as some differentiated embryonic (pre-fetal) cells, show increased iTM and iCM when DIO3 is inhibited either at the transcriptional or protein enzymatic levels.

According to some embodiments, the method further comprising administering to the cells an inhibitor of the DIO3 gene or DIO3 protein. According to some embodiments, the DI03 gene is inhibited by RNAi. A

According to some embodiments, the iTM or iCM factors, e.g., endocrine factors, are administered for at least 1 day, at least 2 days, at least 3 days, at least 4 days, at least 5 days, at least 6 days, at least 7 days, at least 8 days, at least 9 days, at least 10 days, at least 11 days, at least 12 days, at least 13 days, at least 14 days, at least 15 days, at least 16 days, at least 17 days, at least 18 days, at least 19 days, at least 20 days, at least 21 days, at least 22 days, at least 23 days, at least 24 days, at least 25 days, at least 26 days, at least 27 days, at least 28 days, at least 29 days, at least 30 days, at least 31 days, at least 32 days, at least 33 days, at least 34 days, at least 35 days, at least 36 days, at least 37 days, at least 38 days, at least 39 days, or at least 40 days.

According to some embodiments, the iTM or iCM factors, e.g., endocrine factors, are administered for up to 1 day, up to 2 days, up to 3 days, up to 4 days, up to 5 days, up to 6 days, up to 7 days, up to 8 days, up to 9 days, up to 10 days, up to 11 days, up to 12 days, up to 13 days, up to 14 days, up to 15 days, up to 16 days, up to 17 days, up to 18 days, up to 19 days, up to 20 days, up to 21 days, up to 22 days, up to 23 days, up to 24 days, up to 25 days, up to 26 days, up to 27 days, up to 28 days, up to 29 days, up to 30 days, up to 31 days, up to 32 days, up to 33 days, up to 34 days, up to 35 days, up to 36 days, up to 37 days, up to 38 days, up to 39 days, or up to 40 days.

According to some embodiments, the iTM or iCM factors, e.g., endocrine factors, are administered for more than 1 day, more than 2 days, more than 3 days, more than 4 days, more than 5 days, more than 6 days, more than 7 days, more than 8 days, more than 9 days, more than 10 days, more than 11 days, more than 12 days, more than 13 days, more than 14 days, more than 15 days, more than 16 days, more than 17 days, more than 18 days, more than 19 days, more than 20 days, more than 21 days, more than 22 days, more than 23 days, more than 24 days, more than 25 days, more than 26 days, more than 27 days, more than 28 days, more than 29 days, more than 30 days, more than 31 days, more than 32 days, more than 33 days, more than 34 days, more than 35 days, more than 36 days, more than 37 days, more than 38 days, more than 39 days, or more than 40 days.

According to some embodiments, the iTM or iCM factors, e.g., endocrine factors, are administered between 1 day and 40 days, between 2 days and 40 days, between 3 days and 40 days, between 4 days and 40 days, between 5 days and 40 days, between 6 days and 40 days, between 7 days and 40 days, between 8 days and 40 days, between 9 days and 40 days, between 10 days and 40 days, between 11 days and 40 days, between 12 days and 40 days, between 13 days and 40 days, between 14 days and 40 days, between 15 days and 40 days, between 16 days and 40 days, between 17 days and 40 days, between 18 days and 40 days, between 19 days and 40 days, between 20 days and 40 days, between 21 days and 40 days, between 22 days and 40 days, between 23 days and 40 days, between 24 days and 40 days, between 25 days and 40 days, between 26 days and 40 days, between 27 days and 40 days, between 28 days and 40 days, between 29 days and 40 days, between 30 days and 40 days, between 31 days and 40 days, between 32 days and 40 days, between 33 days and 40 days, between 34 days and 40 days, between 35 days and 40 days, between 36 days and 40 days, between 37 days and 40 days, between 38 days and 40 days, 39 days and 40 days, between 1 day and 35 days, between 2 days and 35 days, between 3 days and 35 days, between 4 days and 35 days, between 5 days and 35 days, between 6 days and 35 days, between 7 days and 35 days, between 8 days and 35 days, between 9 days and 35 days, between 10 days and 35 days, between 11 days and 35 days, between 12 days and 35 days, between 13 days and 35 days, between 14 days and 35 days, between 15 days and 35 days, between 16 days and 35 days, between 17 days and 35 days, between 18 days and 35 days, between 19 days and 35 days, between 20 days and 35 days, between 21 days and 35 days, between 22 days and 35 days, between 23 days and 35 days, between 24 days and 35 days, between 25 days and 35 days, between 26 days and 35 days, between 27 days and 35 days, between 28 days and 35 days, between 29 days and 35 days, between 30 days and 35 days, between 31 days and 35 days, between 32 days and 35 days, between 33 days and 35 days, between 34 days and 35 days, between 1 day and 30 days, between 2 days and 30 days, between 3 days and 30 days, between 4 days and 30 days, between 5 days and 30 days, between 6 days and 30 days, between 7 days and 30 days, between 8 days and 30 days, between 9 days and 30 days, between 10 days and 30 days, between 11 days and 30 days, between 12 days and 30 days, between 13 days and 30 days, between 14 days and 30 days, between 15 days and 30 days, between 16 days and 30 days, between 17 days and 30 days, between 18 days and 30 days, between 19 days and 30 days, between 20 days and 30 days, between 21 days and 30 days, between 22 days and 30 days, between 23 days and 30 days, between 24 days and 30 days, between 25 days and 30 days, between 26 days and 30 days, between 27 days and 30 days, between 28 days and 30 days, between 29 days and 30 days, between 1 day and 25 days, between 2 days and 25 days, between 3 days and 25 days, between 4 days and 25 days, between 5 days and 25 days, between 6 days and 25 days, between 7 days and 25 days, between 8 days and 25 days, between 9 days and 25 days, between 10 days and 25 days, between 11 days and 25 days, between 12 days and 25 days, between 13 days and 25 days, between 14 days and 25 days, between 15 days and 25 days, between 16 days and 25 days, between 17 days and 25 days, between 18 days and 25 days, between 19 days and 25 days, between 20 days and 25 days, between 21 days and 25 days, between 22 days and 25 days, between 23 days and 25 days, between 24 days and 25 days, between 1 day and 20 days, between 2 days and 20 days, between 3 days and 20 days, between 4 days and 20 days, between 5 days and 20 days, between 6 days and 20 days, between 7 days and 20 days, between 8 days and 20 days, between 9 days and 20 days, between 10 days and 20 days, between 11 days and 20 days, between 12 days and 20 days, between 13 days and 20 days, between 14 days and 20 days, between 15 days and 20 days, between 16 days and 20 days, between 17 days and 20 days, between 18 days and 20 days, between 19 days and 20 days, between 1 day and 15 days, between 2 days and 15 days, between 3 days and 15 days, between 4 days and 15 days, between 5 days and 15 days, between 6 days and 15 days, between 7 days and 15 days, between 8 days and 15 days, between 9 days and 15 days, between 10 days and 15 days, between 11 days and 15 days, between 12 days and 15 days, between 13 days and 15 days, between 14 days and 15 days, between 1 day and 10 days, between 2 days and 10 days, between 3 days and 10 days, between 4 days and 10 days, between 5 days and 10 days, between 6 days and 10 days, between 7 days and 10 days, between 8 days and 10 days, between 9 days and 10 days, between 1 day and 5 days, between 2 days and 5 days, between 3 days and 5 days, between 4 days and 5 days, between 1 day and 2 days, between 1 days and 3 days, between 1 days and 4 days, between 2 days and 10 days, or between 3 days and 10 days.

### Segmental iTM and iCM Factors

Previously-disclosed segmental iTM and iCM factors, e.g., TR inhibitory genes or factors, or inhibitors of TR activator genes or factors, may be used singly or in combination with other segmental iTM and iCM factors or in combination with global iTM and iCM factors to effect cell maturation wherein a minority of gene expression markers are altered to mature embryonic (pre-fetal) cells to that of later fetal or adult-like cells. Segmental factors may be delivered as proteins, RNA, or genes by a variety of modalities such as with gene therapy vectors, naked DNA, RNA, as well as by other means as previously disclosed. Said previously-disclosed (see PCT International Patent Application No. PCT/US14/40601, titled “Compositions and Methods for Induced Tissue Regeneration in Mammalian Species,” filed Jun. 3, 2014; and PCT International Patent Application No. PCT/US2017/036452, titled “Improved Methods for Detecting and Modulating the Embryonic-Fetal Transition in Mammalian Cells,” filed Jun. 7, 2017, U.S. Provisional Application No. 63/274,734, titled “Methods and Compositions Used to Modify Chromatin Architecture to Regulate Phenotype in Aging and Cancer,” filed Nov. 2, 2021; and U.S. Provisional Application No. 63/256,286, titled “Methods for Modulating the Regenerative Phenotype in Mammalian Cells,” filed Oct. 15, 2021, contents of each of which are incorporated herein by reference). iTM and iCM factors include the genes: ACAT2, C18orf56, CAT, COMT, COX7A1, DYNLT3, ELOVL6, FDPS, IAH1, INSIG1, LOC205251, MAOA, NAALADL1, PSMD5, RPS7, SHMT1, TRIM4, and ZNF280D (see PCT International Patent Application No. PCT/US14/40601, titled “Compositions and Methods for Induced Tissue Regeneration in Mammalian Species,” filed Jun. 3, 2014, contents of each of which are incorporated herein by reference); ADIRF, C10orf11, CAAT, CCDC144B, COMT, COX7A1, KRBOX, LINCO0654, LINC01116, MEG3, MIR4458HG, NAALADL1, PCDHGA2, PCDHGA6, PCDHGA7, PCDHGA9, PCDHGA12, PCDHGB3, PCDHGB5, PLPP7 (PPAPDC3), POMC, PRR34, PRSS3, PTCHD3, PTCHD3P1, SPESP1, TRIM4, USP32P1, ZNF300P1, and ZNF572 (see PCT International Patent Application No. PCT/US2017/036452, titled “Improved Methods for Detecting and Modulating the Embryonic-Fetal Transition in Mammalian Cells,” filed Jun. 7, 2017, contents of which are incorporated herein by reference); LMNA (see U.S. Provisional Application No. 63/274,734, titled “Methods and Compositions Used to Modify Chromatin Architecture to Regulate Phenotype in Aging and Cancer,” filed Nov. 2, 2021, contents of which are incorporated herein by reference); ALS2CR11, ANKRD7, ANKRD65, BACE2, BHMT2, C22orf26, CADPS2, CALHM2, CCDC36, CCDC89, CCDC125, CCDC144B, CLDN11, DDX43, DNAJC15, EGFLAM, ESPNL, FAM24B, FGF7, FKBP9L, FLG-AAS1, FRG1B, GPAT2, GYPE, HENMT1, HIST2H2BA, IRAK4, LINCO2315, LOC283788, LOC100233156, LRRK2, MAP10, MEG8, MEG9, MIRLET7BHG, NKAPL, PAX8-AS1, PRPH2, PRR34-AS1, RP5-1043L13.1, RP11-134021.1, SVIL-AS1, TEKT4P2, ZNF578, ZNF585B, ZNF-736, and ZNF790-AS1 (see U.S. Provisional Application No. 63/256,286, titled “Methods for Modulating the Regenerative Phenotype in Mammalian Cells,” filed Oct. 15, 2021, contents of each of which are incorporated herein by reference.).

According to some embodiments, the iTM and iCM factors, e.g., TR inhibitory genes or factors, or inhibitors of TR activator genes or factors, are administered for at least 1 day, at least 2 days, at least 3 days, at least 4 days, at least 5 days, at least 6 days, at least 7 days, at least 8 days, at least 9 days, at least 10 days, at least 11 days, at least 12 days, at least 13 days, at least 14 days, at least 15 days, at least 16 days, at least 17 days, at least 18 days, at least 19 days, at least 20 days, at least 21 days, at least 22 days, at least 23 days, at least 24 days, at least 25 days, at least 26 days, at least 27 days, at least 28 days, at least 29 days, at least 30 days, at least 31 days, at least 32 days, at least 33 days, at least 34 days, at least 35 days, at least 36 days, at least 37 days, at least 38 days, at least 39 days, or at least 40 days.

According to some embodiments, the iTM and iCM factors, e.g., TR inhibitory genes or factors, or inhibitors of TR activator genes or factors, are administered for up to 1 day, up to 2 days, up to 3 days, up to 4 days, up to 5 days, up to 6 days, up to 7 days, up to 8 days, up to 9 days, up to 10 days, up to 11 days, up to 12 days, up to 13 days, up to 14 days, up to 15 days, up to 16 days, up to 17 days, up to 18 days, up to 19 days, up to 20 days, up to 21 days, up to 22 days, up to 23 days, up to 24 days, up to 25 days, up to 26 days, up to 27 days, up to 28 days, up to 29 days, up to 30 days, up to 31 days, up to 32 days, up to 33 days, up to 34 days, up to 35 days, up to 36 days, up to 37 days, up to 38 days, up to 39 days, or up to 40 days.

According to some embodiments, the iTM and iCM factors, e.g., TR inhibitory genes or factors, or inhibitors of TR activator genes or factors, are administered for more than 1 day, more than 2 days, more than 3 days, more than 4 days, more than 5 days, more than 6 days, more than 7 days, more than 8 days, more than 9 days, more than 10 days, more than 11 days, more than 12 days, more than 13 days, more than 14 days, more than 15 days, more than 16 days, more than 17 days, more than 18 days, more than 19 days, more than 20 days, more than 21 days, more than 22 days, more than 23 days, more than 24 days, more than 25 days, more than 26 days, more than 27 days, more than 28 days, more than 29 days, more than 30 days, more than 31 days, more than 32 days, more than 33 days, more than 34 days, more than 35 days, more than 36 days, more than 37 days, more than 38 days, more than 39 days, or more than 40 days.

RNAi

By way of nonlimiting example, dsRNA is prepared from in vitro transcription reactions (Promega) using PCR-generated templates with flanking T7 promoters, purified by phenol extraction and ethanol precipitation, and annealed after resuspension in water. Intact experimental animals are injected with 4×30 nL dsRNA on three consecutive days following induced tissue injury beginning with the first injection two hours after surgery.

According to some embodiments, nucleic acids encoding RNAi against TR activating factors are administered for up to 1 day, up to 2 days, up to 3 days, up to 4 days, up to 5 days, up to 6 days, up to 7 days, up to 8 days, up to 9 days, up to 10 days, up to 11 days, up to 12 days, up to 13 days, up to 14 days, up to 15 days, up to 16 days, up to 17 days, up to 18 days, up to 19 days, up to 20 days, up to 21 days, up to 22 days, up to 23 days, up to 24 days, up to 25 days, up to 26 days, up to 27 days, up to 28 days, up to 29 days, up to 30 days, up to 31 days, up to 32 days, up to 33 days, up to 34 days, up to 35 days, up to 36 days, up to 37 days, up to 38 days, up to 39 days, or up to 40 days.

According to some embodiments, nucleic acids encoding RNAi against TR activating factors are administered for more than 1 day, more than 2 days, more than 3 days, more than 4 days, more than 5 days, more than 6 days, more than 7 days, more than 8 days, more than 9 days, more than 10 days, more than 11 days, more than 12 days, more than 13 days, more than 14 days, more than 15 days, more than 16 days, more than 17 days, more than 18 days, more than 19 days, more than 20 days, more than 21 days, more than 22 days, more than 23 days, more than 24 days, more than 25 days, more than 26 days, more than 27 days, more than 28 days, more than 29 days, more than 30 days, more than 31 days, more than 32 days, more than 33 days, more than 34 days, more than 35 days, more than 36 days, more than 37 days, more than 38 days, more than 39 days, or more than 40 days.

Segmental iTM and iCM Factor Genes

The iTM or iCM factor genes may be expressed at increased levels in either PSCs or in the derivatives of PSCs using various methods known in the art. By way of nonlimiting examples, said vectors may be lentivirus constructs that constitutively or inducibly express the segmental iTM or iCM factor of interest in a cell line. One such method is to introduce the segmental iTM or iCM factor(s) cultured cells that express an embryonic (pre-fetal) pattern of gene expression using lentivirus. A nonlimiting example is the Genecopoeia vector LPP-C0223Lv205-200 with the transgene of interest to produce purified lentiviral particles containing CMV promotor and 1RES2-eGFP4RES-puroncin Tag. A concentration of 10⁸ TU/ml MOI=10 is obtained.

The method further utilizes the steps of: 1) A polybrene sensitivity (0, 4 and 8 μg/ml) test and puromycin dose response (0, 2, 4, 6, 8, 10 μg/ml) kill curve analysis on both lines is first performed in 96 well plate to determine optimal concentrations of exposure during transduction and clone selection respectively.
2) The cells are harvested in log phase growth and plated in 24 well plates at multiple cell densities in respective growth mediums (2 mL final volume) to determine optimal seeding number so that the wells are ˜80% confluent 24 hours later.
3) Then, once seeding density was determined for each cell line, 24 well plates are set up in triplicate (media control, Polybrene control and lentivirus of interest).
4) Cells are incubated at 37° C., 5% CO₂ for ˜24 hours.
5) Culture media is replaced with fresh polybrene containing media and viral particles added to appropriate wells. Cells ware gently swirled and let stand at room temperature for 30 min.
6) Plates are centrifuged at 800×g for 30 minutes at 32° C. with slow acceleration and slow deceleration. Then the plates are removed carefully without disturbing the cells.
7) The cells are incubated at 37° C., 5% CO₂ for 24-48 h in virus containing media.

Then the viral containing media is removed and cells allowed to incubate for an additional 2 days in fresh culture without selection agent. After 2 days, the cells incubate at 37° C., 5% CO₂ for an additional 4 days in fresh culture medium containing selection agent. The cells with GFP+(top 10%) are selected using a single cell sorter (FACS Melody Cell Sorter, BD) into 96-well plates. The selected clones are expanded into 6 well plates and 10 cm dishes in medium containing selection agent and incubated at 37° C., 5% CO₂ until confluent. The top expressing clones from transduced lines are cryopreserved for later use. To obtain RNA for transcriptomic analysis, cells are thawed and are placed in T-25 flasks and cultured and expanded in their respective growth medium for one week. Then 100,000 cells are seeded on 0.1% gelatin coated cultureware in 6 well plates in their respective medium They are placed in a humidified incubator with 10% CO₂ and 5%02 at 37° C. At confluence they are fasted in DMEM 0.5% FBS for 5 days (3 days then refed fast medium for 2 more days).

They are then lysed with 350 ul RLT lysis buffer (Qiagen) and after using a cell scraper the material is removed and placed in RNase DNase free microfuge tubes. RNA is prepared using Qiagen RNAeasy mini kits (Cat #74104) following manufacturer's directions

One of skill in the art can readily design sequences for RNAi agents, e.g., siRNAs, useful for inhibiting expression of mammalian TR activator genes. In some embodiments, such sequences are selected to minimize “off-target” effects. For example, a sequence that is complementary to a sequence present in TR activator gene mRNA and not present in other mRNAs expressed in a species of interest (or not present in the genome of the species of interest) may be used. Position-specific chemical modifications may be used to reduce potential off-target effects. In some embodiments, at least two different RNAi agents, e.g., siRNAs, targeted to TR activator gene mRNA are used in combination. In some embodiments, a microRNA (which may be an artificially designed microRNA) is used to inhibit TR activator gene expression.

In some embodiments of the invention, TR activator gene expression is inhibited using an antisense molecule comprising a single-stranded oligonucleotide that is perfectly or substantially complementary to mRNA encoding TR activator genes. The oligonucleotide hybridizes to TR activator gene mRNA leading, e.g., to degradation of the mRNA by RNase H or blocking of translation by steric hindrance. In other embodiments of the invention, TR activator gene expression is inhibited using a ribozyme or triplex nucleic acid.

In some embodiments, of the invention, a TR activator inhibits at least one activity of an TR activator protein. TR activator activity can be decreased by contacting the TR activator protein with a compound that physically interacts with the TR activator protein. Such a compound may, for example, alter the structure of the TR activator protein (e.g., by covalently modifying it) and/or block the interaction of the TR activator protein with one or more other molecule(s) such as cofactors or substrates. In some embodiments, inhibition or reduction may be a decrease of at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or at least about 99% of a reference level (e.g., a control level). A control level may be the level of the TR activator that occurs in the absence of the factor. For example, an TR factor may reduce the level of the TR activator protein to no more than 95%, no more than 90%, no more than 85%, no more than 80%, no more than 75%, no more than 70%, no more than 65%, no more than 60%, no more than 55%, no more than 50%, no more than 40%, no more than 30%, no more than 25%, no more than 20%, no more than 10%, or no more than 5% of the level that occurs in the absence of the factor under the conditions tested. In some embodiments, levels of the TR activator are reduced to about 75% or less of the level that occurs in the absence of the factor, under the conditions tested. In some embodiments, levels of the TR activator are reduced to about 50% or less of the level that occurs in the absence of the TR factor, under the conditions tested. In some embodiments, levels of the TR activator are reduced to about 25% or less of the level that occurs in the absence of the TR factor, under the conditions tested. In some embodiments, levels of the TR activator are reduced to about 10% or less of the level that occurs in the absence of the TR factor, under the conditions tested. In some cases the level of modulation (e.g., inhibition or reduction) as compared with a control level is statistically significant. As used herein, “statistically significant” refers to a p-value of less than 0.05, e.g., a p-value of less than 0.025 or a p-value of less than 0.01, using an appropriate statistical test (e.g, ANOVA, t-test, etc.).

In some embodiments of the invention, a compound directly inhibits TR activator proteins, i.e., the compound inhibits TR activator proteins by a mechanism that involves a physical interaction (binding) between the TR activator and the iCM or iTM factor. For example, binding of TR activator to an iCM or iTR factor can interfere with the TR activator's ability to catalyze a reaction and/or can occlude the TR activators active site. A variety of compounds can be used to directly inhibit TR activators. Exemplary compounds that directly inhibit TR activators can be, e.g., small molecules, antibodies, or aptamers.

In some embodiments of the invention, an iCM or iTM factor binds covalently to the TR activator. For example, the compound may modify amino acid residue(s) that are needed for enzymatic activity. In some embodiments, an iTM or iCM factor comprises one or more reactive functional groups such as an aldehyde, haloalkane, alkene, fluorophosphonate (e.g., alkyl fluorophosphonate), Michael acceptor, phenyl sulfonate, methylketone, e.g., a halogenated methylketone or diazomethylketone, fluorophosphonate, vinyl ester, vinyl sulfone, or vinyl sulfonamide, that reacts with an amino acid side chain of TR activators. In some embodiments, an iTM or iCM factor inhibitor comprises a compound that physically interacts with a TR activator, wherein the compound comprises a reactive functional group. In some embodiments, the structure of a compound that physically interacts with the TR activator is modified to incorporate a reactive functional group. In some embodiments, the compound comprises a TR activator substrate analog or transition state analog. In some embodiments, the compound interacts with the TR activator in or near the TR activator active site.

In other embodiments, an iCM or iTM factor binds non-covalently to a TR activator and/or to a complex containing the TR activator and a TR activator substrate. In some embodiments, an iTM or iCM factor binds non-covalently to the active site of a TR activator and/or competes with substrate(s) for access to the TR activator active site. In some embodiments, an iTM or iCM factor binds to the TR activator with a K_{d} of approximately 10⁻³ M or less, e.g., 10⁻⁴ M or less, e.g., 10⁻⁵ M or less, e.g., 10⁻⁶ M or less, 10⁻⁷ M or less, 10⁸ M or less, or 10⁻⁹ M or less under the conditions tested, e.g., in a physiologically acceptable solution such as phosphate buffered saline. Binding affinity can be measured, e.g., using surface plasmon resonance (e.g., with a Biacore system), isothermal titration calorimetry, or a competitive binding assay, as known in the art. In some embodiments, the inhibitor comprises a TR activator substrate analog or transition state analog.

In the case of increasing the activity of TR inhibitors, any one of combination of the TR inhibitor genes listed as iCM or iTM factors herein may be used. The levels of the products of these genes may be introduced using the vectors described herein. The levels of the products of these genes may be introduced using the vectors described herein.

Reporter-Based Screening Assays for iTM and iCM Factors

The invention provides methods for identifying segmental or global iTM and iCM factors using (a) a reporter molecule comprising a readily-detectable marker such as GFP or beta galactosidase whose expression is driven by the promoter of one of the TR inhibitor genes described herein such as that for COX7A1. The invention provides screening assays that involve determining whether a test compound affects the expression of TR inhibitor genes (i.e. induces the expression of fetal or adult-onset gene expression) and/or inhibits the expression of TR activator genes (i.e. inhibits the expression of embryonic (pre-fetal) patterns of gene expression. The invention further provides reporter molecules and compositions useful for practicing the methods. In general, compounds identified using the inventive methods can act by any of mechanism that results in increased or decreased TR activator or inhibitor genes to assay for segmental or global iCM and iTM factors. In the case of the TR inhibitory gene NKAPL, the NKAPL promoter, a promoter sequence flanking the 5′ end of the human gene has been characterized to the position of −756 bases to the ATG translation start codon (Yu, M., et al. Biochimica and Biophysica Acta 1574 (2002) 345-353). Transcription start site of the most cDNAs were observed to be at −55 bases of the translation start codon. Examples of promoter sequences useful in carrying out the present invention are promoters for the genes disclosed in PCT/US14/40601, filed Jun. 3, 2014 and titled “Compositions and Methods for Induced Tissue Regeneration in Mammalian Species”; and PCT/US2017/036452, filed Jun. 7, 2017 and titled “Improved Methods for Detecting and Modulating the Embryonic-Fetal Transition in Mammalian Species” and (see U.S. Provisional Application No. 63/274,736, titled “Methods for the Temporal Regulation of Reprogramming Factors in Mammalian Cells,” filed Nov. 2, 2021, contents of each of which are incorporated herein by reference).

The promoter, as well as the rest of the gene sequence, lays in a CpG island, similarly to the promoters of many housekeeping genes, although the expression of COX7A1 is tissue specific. CpG islands are characterized by the abundance of CG dinucleotides that surpasses that of the average, expected content for the genome, over the span of at least 200 bases. The promoter comprises several regulatory binding site sequences: MEF2 at position −524, as well as three E boxes (characterized as E1, E2, and E3), at, respectively—positions −58, −279 and −585; E box is a DNA binding site (CAACTG) that binds members of the myogenic family of regulatory proteins. Additionally, in the region approximately −95 to −68 bases, there are multiple CG rich segments similar to the one recognized by the transcription factor SpI.

The gene itself, as characterized in GRCh38.p7 primary assembly, occupies 1948 bases between positions 36150922 and 36152869 on Human chromosome 18, and comprises 4 exons interspersed by three introns. Gene sequence, with the promoter sequence is curated at NCBI under locus identifier AF037372.

### Reporter Molecules, Cells, and Membranes

In general, detectable moieties useful in the reporter molecules of the invention include light-emitting or light-absorbing compounds that generate or quench a detectable fluorescent, chemiluminescent, or bioluminescent signal. In some embodiments, activation of TR inhibitor genes or inhibition of TR activator genes causes release of the detectable moiety into a liquid medium, and the signal generated or quenched by the released detectable moiety present in the medium (or a sample thereof) is detected. In some embodiments, the resulting signal causes an alteration in a property of the detectable moiety, and such alteration can be detected, e.g., as an optical signal. For example, the signal may alter the emission or absorption of electromagnetic radiation (e.g., radiation having a wavelength within the infrared, visible or UV portion of the spectrum) by the detectable moiety. In some embodiments, a reporter molecule comprises a fluorescent or luminescent moiety, and a second molecule serves as quencher that quenches the fluorescent or luminescent moiety. Such alteration can be detected using apparatus and methods known in the art.

In many embodiments of the invention, the reporter molecule is a genetically encodable molecule that can be expressed by a cell, and the detectable moiety comprises, e.g., a detectable polypeptide. Thus in some embodiments, the reporter molecule is a polypeptide comprising a fluorescent polypeptides such as green, blue, sapphire, yellow, red, orange, and cyan fluorescent proteins and derivatives thereof (e.g., enhanced GFP); monomeric red fluorescent protein and derivatives such as those known as “mFruits”, e.g., mCherry, mStrawberry, mTomato, etc., and luminescent proteins such as aequorin. (It will be understood that in some embodiments, the fluorescence or luminescence occurs in the presence of one or more additional molecules, e.g., an ion such as a calcium ion and/or a prosthetic group such as coelenterazine.) In some embodiments, the detectable moiety comprises an enzyme that acts on a substrate to produce a fluorescent, luminescent, colored, or otherwise detectable product. Examples of enzymes that may serve as detectable moieties include luciferase; beta-galactosidase; horseradish peroxidase; alkaline phosphatase; etc. (It will be appreciated that the enzyme is detected by detecting the product of the reaction.) In some embodiments, the detectable moiety comprises a polypeptide tag that can be readily detected using a second agent such as a labeled (e.g., fluorescently labeled) antibody. For example, fluorescently labeled antibodies that bind to the HA, Myc, or a variety of other peptide tags are available. Thus the invention encompasses embodiments in which a detectable moiety can be detected directly (i.e., it generates a detectable signal without requiring interaction with a second agent) and embodiments in which a detectable moiety interacts (e.g., binds and/or reacts) with a second agent and such interaction renders the detectable moiety detectable, e.g., by resulting in generation of a detectable signal or because the second agent is directly detectable. In embodiments in which a detectable moiety interacts with a second agent to produce a detectable signal, the detectable moiety may react with the second agent is acted on by a second agent to produce a detectable signal. In many embodiments, the intensity of the signal provides an indication of the amount of detectable moiety present. e.g., in a sample being assessed or in area being imaged. In some embodiments, the amount of detectable moiety is optionally quantified, e.g., on a relative or absolute basis, based on the signal intensity.

The invention provides nucleic acids comprising a sequence that encodes a reporter polypeptide of the invention. In some embodiments, a nucleic acid encodes a precursor polypeptide of a reporter polypeptide of the invention. In some embodiments, the sequence encoding the polypeptide is operably linked to expression control elements (e.g., a promoter or promoter/enhancer sequence) appropriate to direct transcription of mRNA encoding the polypeptide. The invention further provides expression vectors comprising the nucleic acids. Selection of appropriate expression control elements may be based, e.g., on the cell type and species in which the nucleic acid is to be expressed. One of ordinary skill in the art can readily select appropriate expression control elements and/or expression vectors. In some embodiments, expression control element(s) are regulatable, e.g., inducible or repressible. Exemplary promoters suitable for use in bacterial cells include, e.g., Lac, Trp, Tac, araBAD (e.g., in a pBAD vectors), phage promoters such as T7 or T3. Exemplary expression control sequences useful for directing expression in mammalian cells include, e.g., the early and late promoters of SV40, adenovirus or cytomegalovirus immediate early promoter, or viral promoter/enhancer sequences, retroviral LTRs, promoters or promoter/enhancers from mammalian genes, e.g., actin, EF-1 alpha, phosphoglycerate kinase, etc. Regulatable (e.g., inducible or repressible) expression systems such as the Tet-On and Tet-Off systems (regulatable by tetracycline and analogs such as doxycycline) and others that can be regulated by small molecules such as hormones receptor ligands (e.g., steroid receptor ligands, which may or may not be steroids), metal-regulated systems (e.g., metallothionein promoter), etc.

The invention further provides cells and cell lines that comprise such nucleic acids and/or vectors. In some embodiments, the cells are eukaryotic cells, e.g., fungal, plant, or animal cells. In some embodiments, the cell is a vertebrate cell, e.g., a mammalian cell, e.g., a human cell, non-human primate cell, or rodent cell. Often a cell is a member of a cell line, e.g., an established or immortalised cell line that has acquired the ability to proliferate indefinitely in culture (e.g., as a result of mutation or genetic manipulation such as the constitutive expression of the catalytic component of telomerase). Numerous cell lines are known in the art and can be used in the instant invention. Mammalian cell lines include, e.g., HEK-293 (e.g., HEK-293T), CHO, NIH-3T3, COS, HT1080, and HeLa cell lines. In some embodiments, a cell line is a tumor cell line. In other embodiments, a cell is non-tumorigenic and/or is not derived from a tumor. In some embodiments, the cells are adherent cells. In some embodiments, non-adherent cells are used. In some embodiments, a cell is of a cell type or cell line is used that has been shown to naturally have a subset of TR activator genes expressed or TR inhibitor genes not expressed. If a cell lacks one or more TR activator or inhibitor genes, the cell can be genetically engineered to express such protein(s). In some embodiments, a cell line of the invention is descended from a single cell. For example, a population of cells can be transfected with a nucleic acid encoding the reporter polypeptide and a colony derived from a single cell can be selected and expanded in culture. In some embodiments, cells are transiently transfected with an expression vector that encodes the reporter molecule. Cells can be co-transfected with a control plasmid, optionally expressing a different detectable polypeptide, to control for transfection efficiency (e.g., across multiple runs of an assay).

### iCM and iTM Activator and TR Inhibitor Polypeptides and Nucleic Acids

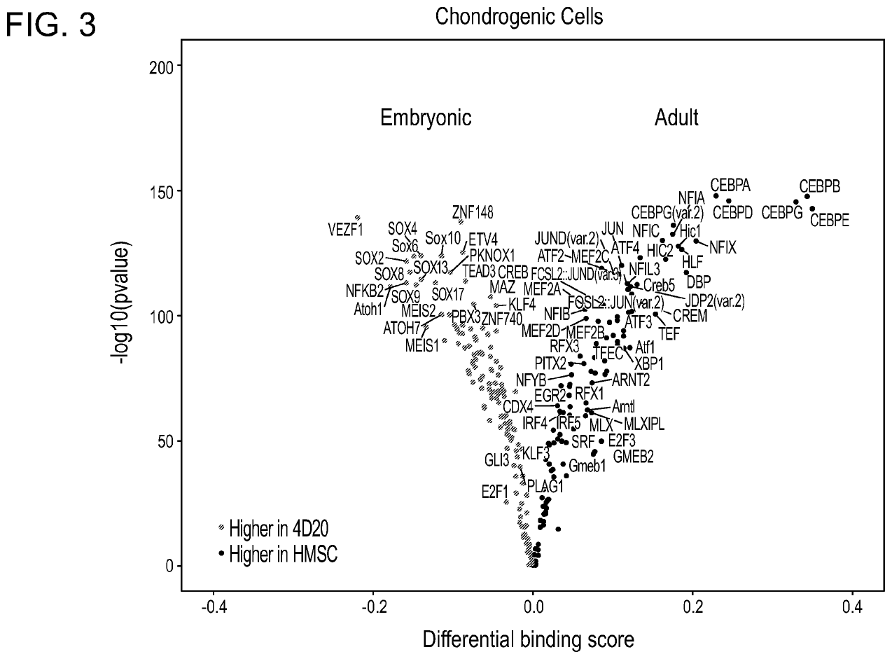

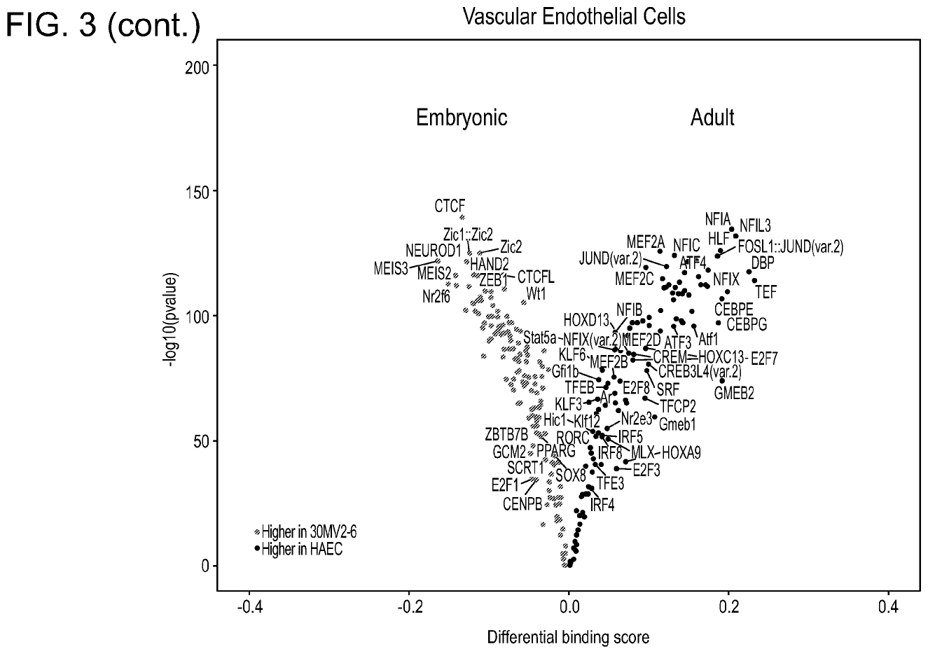

Segmental iCM or iTM factors include the TR inhibitory genes including combinations of: ACAT2, C18orf56, CAT, COMT, COX7A1, DYNLT3, ELOVL6, FDPS, IAH1, INSIG1, LOC205251, MAOA, RPS7, SHMT1, TRIM4, TSPYL5, or ZNF280D (see previously disclosed in PCT/US14/40601, filed Jun. 3, 2014 and titled “Compositions and Methods for Induced Tissue Regeneration in Mammalian Species,”) or ADIRF, C10orf11, CAT, CCDC144B, COMT, COX7A1, KRBOX1, LINC00654, LINC00839, LINC01116, MEG3, MIR4458HG, PCDHGA12, PCDHGB3, PCDHGB5, PLPP7 (PPAPDC3), POMC, PRR34, PRSS3, PTCHD3, PTCHD3P1, SPESP1, TRIM4, USP32P1, ZNF300P1, or ZNF572 (see previously disclosed in PCT/US2017/036452, filed Jun. 7, 2017 and titled “Improved Methods for Detecting and Modulating the Embryonic-Fetal Transition in Mammalian Species,”) or ALS2CR11, C2CD6, ANKRD7, ANKRD65, BACE2, BHMT2, C22orf26, CADPS2, CALHM2, CCDC36, CCDC89, CCDC125, CCDC144B, CLDN11, CTSF, DDX43, DNAJC15, EGFLAM, ESPNL, FAM24B, FGF7, FKBP9L, FLG-AS1, FRG1B, GPAT2, GYPE, HENMT1, HIST2H2BA, IRAK4, LINC00865, LOC283788, LOC100233156, LRRK2, MAP10, MEG8, MEG9, MIRLET7HG, NKAPL, PAX8-AS1, PRPH2, PRR34-AS1, RP5-1043L13.1, RP11-134021.1, SVIL-AS1, TEKT4P2, ZNF578, ZNF585B, ZNF736, or ZNF790-AS1 previously disclosed in U.S. Provisional Application No. 63/256,286, titled “Methods for Modulating the Regenerative Phenotype in Mammalian Cells,” filed Oct. 15, 2021 Additionally, transcriptional regulators disclosed herein capable of inducing iTM and iCM include members of the AP-1 transcription factor complex such as FOS, JUN, and JUND or other transcriptional regulators up-regulated in the majority of adult cells compared to embryonic (pre-fetal) cells such as those encoded by the genes: ATF2, ATF4, CEBPB, CEBPE, CEBPG, FOSL1, FOSL2, HIC1, HIC2, MEF2B, NFIA, NFIC, and NFIX (FIG. 3).

Segmental iTM or segmental iCM may also be accomplished by means of the down-regulation of the expression or otherwise reduction of the transcripts for TR activating genes. By way of non-limiting example, said down-regulation of TR activating genes may be achieved by the of RNAi. Examples of such TR activator genes include: AFF3, CBCAQH03 5, DLX1, DRD11P, F2RL2, FOXD1, LOC728755, LOC791120, MN1, OXTR, PCDHB2, PCDHB17, RAB3IP, SIX1, and WSB1 (see PCT/US14/40601, filed Jun. 3, 2014 and titled “Compositions and Methods for Induced Tissue Regeneration in Mammalian Species”); the genes: ADGRV1, AFF3, ALDH5A1, ALX1, AMH, B4GALNT4, C14orf39, CHKB-CPT1B, CPT1B, DOC2GP, DPY19L2, DSG2, FAM157A, FAM157B, FOXD4L4, FSIP2, GDF1, GRIN3B, H2BFXP, L3MBTL1, LIN28B, LINC00649, LINC01021, LINC01116, NAALAD2, PAQR6, members of the alpha clustered protocadherin locus A2-11, members of the beta clustered protocadherin locus B2-17, PCDHGB4, PCDHGB6, PLPPR3, PRR5L, RGPD1, SLCO1A2, TSPAN11, TUBB2B, ZCCHC18, ZNF497, and ZNF853 (see PCT/US2017/036452, filed Jun. 7, 2017 and titled “Improved Methods for Detecting and Modulating the Embryonic-Fetal Transition in Mammalian Species,”); and the gene LMNB1 (see U.S. Provisional Application No. 63/274,734, titled “Methods and Compositions Used to Modify Chromatin Architecture to Regulate Phenotype in Aging and Cancer,” filed Nov. 2, 2021, contents of each of which are incorporated herein by reference); and the genes: AC108142.1, AGA, AQP7P1, AQP7P3, BAHD1, BBOX1, C11orf35 (LMNTD2), CASC9, CBX2, CCDC144NL, CHRM3, CPAMD8, FAR2P1, FAR2P2, FAR2P3, FIRRE, IGF2BP1, LINC00649, LINC02315, LOC644919, MED15P9, PCAT7, PKP3, POTEE, POTEF, PURPL, RGPD2, WDR72, or WRN (see U.S. Provisional Application No. 63/256,286, titled “Methods for Modulating the Regenerative Phenotype in Mammalian Cells,” filed Oct. 15, 2021 contents of each of which are incorporated herein by reference).

Polypeptides may also be utilized in the manufacture of iCM and iTM factors. TR inhibitor polypeptides useful in producing a segmental iCM or iTM outcome may be obtained by a variety of methods. In some embodiments, the polypeptides are produced using recombinant DNA techniques. Standard methods for recombinant protein expression can be used. A nucleic acid encoding a TR inhibitor gene can readily be obtained, e.g., from cells that express the genes (e.g., by PCR or other amplification methods or by cloning) or by chemical synthesis or in vitro transcription based on the cDNA sequence polypeptide sequence. One of ordinary skill in the art would know that due to the degeneracy of the genetic code, the genes can be encoded by many different nucleic acid sequences. Optionally, a sequence is codon-optimized for expression in a host cell of choice. The genes could be expressed in bacterial, fungal, animal, or plant cells or organisms. The genes could be isolated from cells that naturally express it or from cells into which a nucleic acid encoding the protein has been transiently or stably introduced, e.g., cells that contain an expression vector encoding the genes. In some embodiments, the gene is secreted by cells in culture and isolated from the culture medium.

In some embodiments of the invention, the sequence of a TR inhibitor polypeptide is used in the inventive screening methods. A naturally occurring TR inhibitor polypeptide can be from any species whose genome encodes a TR inhibitor polypeptide, e.g., human, non-human primate, rodent, etc. A polypeptide whose sequence is identical to naturally occurring TR inhibitor is sometimes referred to herein as “native TR inhibitor”. A TR inhibitor polypeptide of use in the invention may or may not comprise a secretion signal sequence or a portion thereof. For example, mature TR inhibitor comprising or consisting of amino acids 20-496 of human TR inhibitor (or corresponding amino acids of TR inhibitor of a different species) may be used.

In some embodiments, a polypeptide comprising or consisting of a variant or fragment of TR inhibitor is used. TR inhibitor variants include polypeptides that differ by one or more amino acid substitutions, additions, or deletions, relative to TR inhibitor. In some embodiments, a TR inhibitor variant comprises a polypeptide at least 70%, 75%, 80%, 85%, 90%, 95%, 96%, 97%, 98%, 99%, or more identical to at least amino acids full length TR inhibitor (e.g., from human or mouse), or over at least 50%, 60%, 70%, 80%, 85%, 90%, 95%, 96%, 97%, 98%, 99%, or 100% of full length human TR inhibitor or of full length mouse TR inhibitor. In some embodiments, a TR inhibitor variant comprises a polypeptide at least 70%, 75%, 80%, 85%, 90%, 95%, 96%, 97%, 98%, 99%, or more identical to at least amino acids 20-496 of TR inhibitor (e.g., from human or mouse) over at least 50%, 60%, 70%, 80%, 85%, 90%, 95%, 96%, 97%, 98%, 99%, or 100% of at least amino acids 20-496 of human TR inhibitor or amino acids 20-503 of mouse TR inhibitor. In some embodiments, a TR inhibitor variant comprises a polypeptide at least 80%, 85%, 90%, 95%, 96%, 97%, 98%, 99%, or more identical to at least amino acids 20-496 of human TR inhibitor or amino acids 20-503 of mouse TR inhibitor. In some embodiments, a TR inhibitor polypeptide comprises a polypeptide at least 80%, 85%, 90%, 95%, 96%, 97%, 98%, 99%, or more identical to at least amino acids 20-496 of human TR inhibitor or amino acids 20-503 of mouse TR inhibitor. A nucleic acid that encodes a TR inhibitor variant or fragment can readily be generated, e.g., by modifying the DNA that encodes native TR inhibitor using, e.g., site-directed mutagenesis, or by other standard methods, and used to produce the TR inhibitor variant or fragment. For example, a fusion protein can be produced by cloning sequences that encode TR inhibitor into a vector that provides the sequence encoding the heterologous portion. In some embodiments a tagged TR inhibitor is used. For example, in some embodiments a or TR inhibitor polypeptide comprising a His tag, e.g., at its C terminus, is used.

### Test Compounds

A wide variety of test compounds can be used in the inventive methods for identifying iTM or iCM factors and global modulators of iTM or iCM. For example, a test compound can be a small molecule, polypeptide, peptide, nucleic acid, oligonucleotide, lipid, carbohydrate, antibody, or hybrid molecule including but not limited to those described herein, including mRNA for the genes COX7A1 and LAMNA alone and in diverse combinations, and in diverse combinations with endocrine factors including T3, T4, dexamethasone, FGF7, cortisol, and growth hormone.

In vitro assays for iTM or iCM patterns of expression of the genes COX7A1, PCDHGA12, COMT, or TRIM4 or in the case of iCM, the expression of CSC markers or mesenchymal markers such as (CD44, ALDH1A1, ALCAM, CD133) or (COL1A1, SPARC, VIM, FN1), respectively, are performed to optimize a formulation for global patterns of iTM and iCM gene expression. Assays may be performed at various time points following exposure to iCM or iTM factors such as assays performed at 0 day, 1 day, 2 days, 3 days, 4 days, 5 days, 6 days, 7 days, 8 days, 9 days, 10 days, 11 days, 12 days, 13 days, 14 days, 15 days, 16, days, 17 days, 18 days, 19 days, and 20 days for markers of global modulation of iTM or iCM gene expression. Assays may be performed at various time points following exposure to iCM or iTM factors such as assays performed for at least 1 days, at least 2 days, at least 3 days, at least 4 days, at least 5 days, at least 6 days, at least 7 days, at least 8 days, at least 9 days, at least 10 days, at least 11 days, at least 12 days, at least 13 days, at least 14 days, at least 15 days, at least 17 days, at least 18 days, at least 19 days, and at least 20 days for markers of global modulation of iTM or iCM gene expression. Assays may be performed at various time points following exposure to iCM or iTM factors such as assays performed for up to 1 day, up to 2 days, up to 3 days, up to 4 days, up to 5 days, up to 6 days, up to 7 days, up to 8 days, up to 9 days, up to 10 days, up to 11 days, up to 12 days, up to 13 days, up to 14 days, up to 15 days, up to 16 days, up to 17 days, up to 18 days, up to 19 days, and up to 20 days for markers of global modulation of iTM or iCM gene expression.

### Improved iTR Factor Screen

The screen for compounds or combinations of compounds useful in producing either a global or segmental iTM or iCM effect in cells with an embryonic pattern of gene expression whether as a result of being derived from PSCs or cancer cell lines may be implemented in vitro or in vivo (such as animal models). Preferably said screen is performed in vitro in low- or high-throughput. The screen assays for increase or decrease of embryonic or fetal/adult markers respectively. Said screen may employ the use of antibodies, the assay of RNA, metabolic markers, or other markers described herein or described in “Compositions and Methods for Induced Tissue Regeneration in Mammalian Species” (International Patent Application publication number WO 2014/197421), incorporated herein by reference in its entirety and “Improved Methods for Detecting and Modulating the Embryonic-Fetal Transition in Mammalian Species” (International Patent Application publication number WO 2017/214342, incorporated herein by reference in its entirety). Preferably, said screens assay the levels of mRNA markers, most preferably an increase in the expression of COX7A1 as an indicator of iTM or iCM. Most preferably said screen is performed in multi-well format and the expression is assays through the use of a reporter gene such as eGFP knocked-in to the COX7A1 locus in the genome.

Compounds can be obtained from natural sources or produced synthetically. Compounds can be at least partially pure or may be present in extracts or other types of mixtures whose components are at least in part unknown or uncharacterized. Extracts or fractions thereof can be produced from, e.g., plants, animals, microorganisms, marine organisms, fermentation broths (e.g., soil, bacterial or fungal fermentation broths), etc. In some embodiments, a compound collection (“library”) is tested. The library may comprise, e.g., between 100 and 500,000 compounds, or more. Compounds are often arrayed in multiwell plates (e.g., 384 well plates, 1596 well plates, etc.). They can be dissolved in a solvent (e.g., DMSO) or provided in dry form, e.g., as a powder or solid. Collections of synthetic, semi-synthetic, and/or naturally occurring compounds can be tested. Compound libraries can comprise structurally related, structurally diverse, or structurally unrelated compounds. Compounds may be artificial (having a structure invented by man and not found in nature) or naturally occurring. In some embodiments, a library comprises at least some compounds that have been identified as “hits” or “leads” in other drug discovery programs and/or derivatives thereof. A compound library can comprise natural products and/or compounds generated using non-directed or directed synthetic organic chemistry. Often a compound library is a small molecule library. Other libraries of interest include peptide or peptoid libraries, cDNA libraries, antibody libraries, and oligonucleotide libraries. A library can be focused (e.g., composed primarily of compounds having the same core structure, derived from the same precursor, or having at least one biochemical activity in common).

Compounds chosen for screening may be chosen from a library of synthetic or natural product-derived small molecules with a variety of chemical structures known to provide potential bioactive motifs. Preferably said compounds are known activators of the MEK/ERK pathway, or are chemically-related to said compounds.

Compound libraries are available from a number of commercial vendors such as Tocris BioScience, Nanosyn, BioFocus, and from government entities. For example, the Molecular Libraries Small Molecule Repository (MLSMR), a component of the U.S. National Institutes of Health (NIH) Molecular Libraries Program is designed to identify, acquire, maintain, and distribute a collection of >300,000 chemically diverse compounds with known and unknown biological activities for use, e.g., in high-throughput screening (HTS) assays (see www.mli.nih.gov/mli/). The NIH Clinical Collection (NCC) is a plated array of approximately 450 small molecules that have a history of use in human clinical trials. These compounds are highly drug-like with known safety profiles. In some embodiments, a collection of compounds comprising “approved human drugs” is tested. An “approved human drug” is a compound that has been approved for use in treating humans by a government regulatory agency such as the US Food and Drug Administration, European Medicines Evaluation Agency, or a similar agency responsible for evaluating at least the safety of therapeutic agents prior to allowing them to be marketed. The test compound may be, e.g., an antineoplastic, antibacterial, antiviral, antifungal, antiprotozoal, antiparasitic, antidepressant, antipsychotic, anesthetic, antianginal, antihypertensive, antiarrhythmic, antiinflammatory, analgesic, antithrombotic, antiemetic, immunomodulator, antidiabetic, lipid- or cholesterol-lowering (e.g., statin), anticonvulsant, anticoagulant, antianxiety, hypnotic (sleep-inducing), hormonal, or anti-hormonal drug, etc. In some embodiments, a compound is one that has undergone at least some preclinical or clinical development or has been determined or predicted to have “drug-like” properties. For example, the test compound may have completed a Phase I trial or at least a preclinical study in non-human animals and shown evidence of safety and tolerability.

In some embodiments, a test compound is substantially non-toxic to cells of an organism to which the compound may be administered and/or to cells with which the compound may be tested, at the concentration to be used or, in some embodiments, at concentrations up to 10-fold, 100-fold, or 1,000-fold higher than the concentration to be used. For example, there may be no statistically significant effect on cell viability and/or proliferation, or the reduction in viability or proliferation can be no more than 1%, 5%, or 10% in various embodiments. Cytotoxicity and/or effect on cell proliferation can be assessed using any of a variety of assays. For example, a cellular metabolism assay such as AlamarBlue, MTT, MTS, XTT, and CellTitre Glo assays, a cell membrane integrity assay, a cellular ATP-based viability assay, a mitochondrial reductase activity assay, a BrdU, EdU, or H3-Thymidine incorporation assay could be used. In some embodiments, a test compound is not a compound that is found in a cell culture medium known or used in the art, e.g., culture medium suitable for culturing vertebrate, e.g., mammalian cells or, if the test compound is a compound that is found in a cell culture medium known or used in the art, the test compound is used at a different, e.g., higher, concentration when used in a method of the present invention.

Assays for Global modulators of iTR

### Aspects of Assay Implementation and Controls

Various inventive screening assays described above involve determining whether a test compound inhibits the levels of active TR activators or increases the levels of active TR inhibitors. Suitable cells for expression of a reporter molecule are described above.

In performing an inventive assay, assay components (e.g., cells, iTM or iCM activator or TR inhibitor polypeptide, global iTM or global iCM activators, or other test compounds) are typically dispensed into multiple vessels or other containers. Any type of vessel or article capable of containing cells can be used. In many embodiments of the invention, the vessels are wells of a multi-well plate (also called a “microwell plate”, “microtiter plate”, etc. For purposes of description, the term “well” will be used to refer to any type of vessel or article that can be used to perform an inventive screen, e.g., any vessel or article that can contain the assay components. It should be understood that the invention is not limited to use of wells or to use of multi-well plates. In some embodiments, any article of manufacture in which multiple physically separated cavities (or other confining features) are present in or on a substrate can be used. For example, assay components can be confined in fluid droplets, which may optionally be arrayed on a surface and, optionally, separated by a water-resistant substance that confines the droplets to discrete locations, in channels of a microfluidic device, etc.

In general, assay components can be added to wells in any order. For example, cells can be added first and maintained in culture for a selected time period (e.g., between 2 and 48 hours) prior to addition of a test compound and target TR activator or TR inhibitor polypeptides or cells with express constructs to a well. In some embodiments, compounds are added to wells prior to addition of polypeptides or cells. In some embodiments, expression of a reporter polypeptide is induced after plating the cells, optionally after addition of a test compound to a well. In some embodiments, expression of the reporter molecule is achieved by transfecting the cells with an expression vector that encodes the reporter polypeptide. In some embodiments, the cells have previously been genetically engineered to express the reporter polypeptide. In some embodiments, expression of the reporter molecule is under control of regulatable expression control elements, and induction of expression of the reporter molecule is achieved by contacting the cells with an agent that induces (or derepresses) expression.

The assay composition comprising cells, test compound, or polypeptide is maintained for a suitable time period during which test compound may (in the absence of a test compound that modulates its activity) cause an increase of the level or activity of the target TR inhibitor. The number of cells, amount of polypeptide, and amount of test compound to be added will depend, e.g., on factors such as the size of the vessel, cell type, and can be determined by one of ordinary skill in the art. In some embodiments, the ratio of the molar concentration of TR inhibitor polypeptide to test compound is between 1:10 and 10:1. In some embodiments, the number of cells, amount of test compound, and length of time for which the composition is maintained can be selected so that a readily detectable level signal after a selected time period in the absence of a test compound. In some embodiments, cells are at a confluence of about 25%-75%, e.g., about 50%, at the time of addition of compounds. In some embodiments, between 1,000 and 10,000 cells/well (e.g., about 5,000 cells/well) are plated in about 100 μl medium per well in 96-well plates. In other exemplary embodiments, cells are seeded in about 30 μl-50 μl of medium at between 500 and 2,000 (e.g., about 1000) cells per well into 384-well plates. In some embodiments, compounds are tested at multiple concentrations (e.g., 2-10 different concentrations) and/or in multiple replicates (e.g., 2-10 replicates). Multiple replicates of some or all different concentrations can be performed. In some embodiments, candidate iCM or iTM factors are used at a concentration between 0.1 ug/ml and 100 μg/ml, e.g., 1 μg/ml and 10 μg/ml. In some embodiments, candidate iCM or iTM factors are used at multiple concentrations. In some embodiments, compounds are added to cells between 6 hours and one day (24 hr) after seeding.

In some aspects of any of the inventive compound screening and/or characterization methods, a test compound is added to an assay composition in an amount sufficient to achieve a predetermined concentration. In some embodiments the concentration is up to about 1 nM. In some embodiments the concentration is between about 1 nM and about 100 nM. In some embodiments the concentration is between about 100 nM and about 10 M. In some embodiments the concentration is at least 10 M, e.g., between 10 M and 100 M. The assay composition can be maintained for various periods of time following addition of the last component thereof. In certain embodiments the assay composition is maintained for between about 10 minutes and about 4 days, e.g., between 1 hour and 3 days, e.g., between 2 hours and 2 days, or any intervening range or particular value, e.g., about 4-8 hours, after addition of all components. In certain embodiments, the assay composition is maintained for between about 10 minutes and about 20 days, e.g., between 1 hour and 12 days, e.g., between 2 hours and 5 days, or any intervening range or particular value, e.g., about 10-24 hours, after addition of all components. Multiple different time points can be tested. Additional aliquots of test compound can be added to the assay composition within such time period. In some embodiments, cells are maintained in cell culture medium appropriate for culturing cells of that type. In some embodiments, a serum-free medium is used. In some embodiments, the assay composition comprises a physiologically acceptable liquid that is compatible with maintaining integrity of the cell membrane and, optionally, cell viability, instead of cell culture medium. Any suitable liquid could be used provided it has the proper osmolarity and is otherwise compatible with maintaining reasonable integrity of the cell membrane and, optionally, cell viability, for at least a sufficient period of time to perform an assay. One or more measurements indicative of an increase in the level of active TR inhibitor can be made during or following the incubation period.

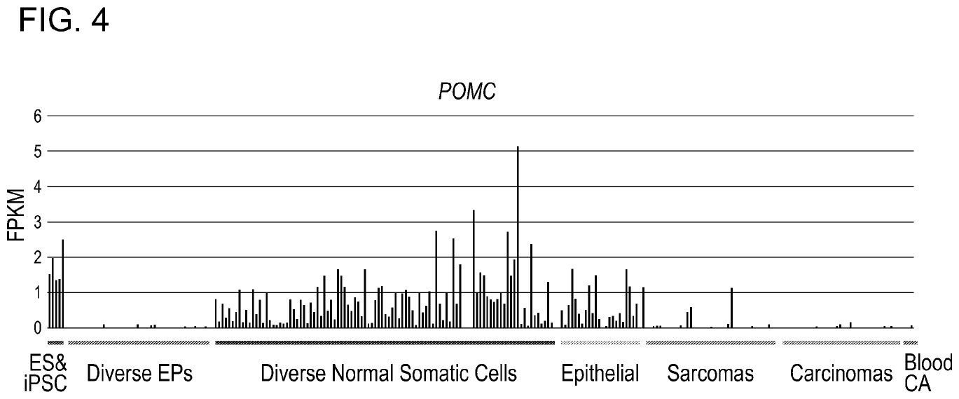

In some embodiments, the compounds screened for potential to be global modulators of iTM or iCM are chosen from agents capable of activating the ERK1/2 signaling pathway resulting in increased levels or activity, such as through altered post-translational modification, by way of nonlimiting example, phosphorylation) of components of the AP-1 complex, (FOS, JUN, JUND) or other transcriptional regulators up-regulated in the majority of adult cells compared to embryonic (pre-fetal) cells such as those encoded by the genes: ATF2, ATF4, CEBPB, CEBPE, CEBPG, FOSL1, FOSL2, HIC1, HIC2, MEF2B, NFIA, NFIC, and NFIX, whose relative abundance positioned on chromatin in embryonic vs adult cells is shown in FIG. 3. Said compounds are, by way of nonlimiting example, thyroid hormones T3 and T4, preferably T3, adrenal steroids such as cortisol or the synthetic analog dexamethasone, FGF7, growth hormone (GH), or proopiomelanocortin (POMC) or and POMC derivative peptides (e.g., N-terminal peptide of proopiomelanocortin (NPP), alpha melanotropin ((αMSH), beta-melanotropin (βMSH), delta-melanocyte-stimulating hormone (δMSH), epsilson-melanocyte-stimulating hormone (εMSH), corticotropin, beta-lipotropin, gamma lipotropin, beta-endorphin, and met-enkephalin) thyroid stimulating hormone and corticotropin. POMC RNA is markedly up-regulated in adult cells compared to embryonic (pre-fetal) cells and markedly down-regulated in the majority of cancer cell lines (FIG. 4).

In some embodiments, individual compounds, each typically of known identity (e.g., structure and/or sequence), are added to each of a multiplicity of wells. In some embodiments, two or more compounds may be added to one or more wells. In some embodiments, one or more compounds of unknown identity may be tested. The identity may be determined subsequently using methods known in the art.

In various embodiments, foregoing assay methods of the invention are amenable to high-throughput screening (HTS) implementations. In some embodiments, the screening assays of the invention are high throughput or ultra high throughput (see, e.g., Fernandes, P. B., Curr Opin Chem. Biol. 1998, 2:597; Sundberg, S A, Curr Opin Biotechnol. 2000, 11:47). High throughput screens (HTS) often involve testing large numbers of compounds with high efficiency, e.g., in parallel. For example, tens or hundreds of thousands of compounds can be routinely screened in short periods of time, e.g, hours to days. In some embodiments, HTS refers to testing of between 1,000 and 100,000 compounds per day. In some embodiments, ultra high throughput refers to screening in excess of 100,000 compounds per day, e.g., up to 1 million or more compounds per day. The screening assays of the invention may be carried out in a multi-well format, for example, a 96-well, 384-well format, 1,536-well format, or 3,456-well format and are suitable for automation. In some embodiments, each well of a microwell plate can be used to run a separate assay against a different test compound or, if concentration or incubation time effects are to be observed, a plurality of wells can contain test samples of a single compound, with at least some wells optionally being left empty or used as controls or replicates. Typically, HTS implementations of the assays disclosed herein involve the use of automation. In some embodiments, an integrated robot system including one or more robots transports assay microwell plates between multiple assay stations for compound, cell and/or reagent addition, mixing, incubation, and readout or detection. In some aspects, an HTS system of the invention may prepare, incubate, and analyze many plates simultaneously. Suitable data processing and control software may be employed. High throughput screening implementations are well known in the art. Without limiting the invention in any way, certain general principles and techniques that may be applied in embodiments of a HTS of the present invention are described in Macarron R & Hertzberg R P. Design and implementation of high-throughput screening assays. Methods Mol Biol., 565:1-32, 2009 and/or An W F & Tolliday N J., Introduction: cell-based assays for high-throughput screening. Methods Mol Biol. 486:1-12, 2009, and/or references in either of these. Exemplary methods are also disclosed in High Throughput Screening: Methods and Protocols (Methods in Molecular Biology) by William P. Janzen (2002) and High-Throughput Screening in Drug Discovery (Methods and Principles in Medicinal Chemistry) (2006).

An additional compound may, for example, have one or more improved pharmacokinetic and/or pharmacodynamic properties as compared with an initial hit or may simply have a different structure. An “improved property” may, for example, render a compound more effective or more suitable for one or more purposes described herein. In some embodiments, for example, a compound may have higher affinity for the molecular target of interest (e.g., TR inhibitor gene products), lower affinity for a non-target molecule, greater solubility (e.g., increased aqueous solubility), increased stability (e.g., in blood, plasma, and/or in the gastrointestinal tract), increased half-life in the body, increased bioavailability, and/or reduced side effect(s), etc. Optimization can be accomplished through empirical modification of the hit structure (e.g., synthesizing compounds with related structures and testing them in cell-free or cell-based assays or in non-human animals) and/or using computational approaches. Such modification can in some embodiments make use of established principles of medicinal chemistry to predictably alter one or more properties. In some embodiments, one or more compounds that are “hit” are identified and subjected to systematic structural alteration to create a second library of compounds (e.g., refined lead compounds) structurally related to the hit. The second library can then be screened using any of the methods described herein.

In some embodiments, an iTM or iCM factor is modified or incorporates a moiety that enhances stability (e.g., in serum), increases half-life, reduces toxicity or immunogenicity, or otherwise confers a desirable property on the compound.

### Uses of and iTM Factors

### Pharmaceutical Compositions

Since the majority of mammalian somatic cells undergo maturation at or around the EFT and since said maturation is associated with fetal and adult marker expression as disclosed herein regardless of the differentiated cell type, iCM, and iTM factors have a variety of different uses. Non-limiting examples of such uses are discussed herein. In some embodiments, an iTM factor is used to mature PSC-derived cells with an embryonic (pre-fetal) pattern of gene expression. Said immature cells with an embryonic pattern of gene expression are any somatic cell type, even cell types that when mature express relatively high levels of COX7A1 RNA, for instance PSC-derived muscle cells, or brown adipocytes (FIGS. 2A-2D). The relatively high expression of CPT1B in embryonic cells and the relatively high expression of COX7A1 in adult cells results in profound differences in the metabolic state of cells for example. In the case of drug screening, to obtain cells with normal adult physiology and metabolism, iTM provides a useful means of producing large quantities of mature cell types for in vitro assays.

The iTM applications for non-cancerous cell types include the maturation of PSC-derived cells such that the cells, by way of nonlimiting example, express increased levels of the adult markers COX7A1 or PCDHGA12. Such relatively developmentally-mature cells may then be used in transplantation wherein mature cells have advantages in treating disease. Examples may include the maturation of pancreatic beta cells such that the cells regulate blood glucose at adult physiological levels. Other applications include the maturation and then subsequent transplantation of the cells to enhance regeneration of a limb, digit, cartilage, heart, blood vessel, bone, esophagus, stomach, liver, gallbladder, pancreas, intestines, rectum, anus, endocrine gland (e.g., thyroid, parathyroid, adrenal, endocrine portion of pancreas), skin, hair follicle, thymus, spleen, skeletal muscle, focal damaged cardiac muscle, smooth muscle, brain, spinal cord, peripheral nerve, ovary, fallopian tube, uterus, vagina, mammary gland, testes, vas deferens, seminal vesicle, prostate, penis, pharynx, larynx, trachea, bronchi, lungs, kidney, ureter, bladder, urethra, eye (e.g., retina, cornea), or ear (e.g., organ of Corti). In some embodiments, said matured cells are used to enhance regeneration of a stromal layer, e.g., a connective tissue supporting the parenchyma of a tissue.

Enhancing regeneration using developmentally-mature cells can include any one or more of the following, in various embodiments: (a) increasing the extant of engraftment in a mammalian tissue; (b) increasing the extent of proliferation of the cells; (c) promoting connective tissue and associated extracellular matrix of the engrafted cells for use in repairing tissues with a large component of extracellular matrix such as tendon.

Numerous aspects of aging and age-related disease are taught in the present invention to addressable with iTM therapy. These manifestations of aging include age-related vascular dysfunction including peripheral vascular, coronary, and cerebrovascular disease; musculoskeletal disorders including osteoarthritis, intervertebral disc degeneration, bone fractures, tendon and ligament tears, and limb regeneration; neurological disorders including stroke and spinal cord injuries; muscular disorders including muscular dystrophy, sarcopenia, myocardial infarction, and heart failure; endocrine disorders including Type I diabetes, Addison's disease, hypothyroidism, and pituitary insufficiency; digestive disorders including pancreatic exocrine insufficiency; ocular disorders including macular degeneration, retinitis pigmentosa, and neural retinal degeneration disorders; dermatological conditions including skin burns, lacerations, surgical incisions, alopecia, graying of hair, and skin aging; pulmonary disorders including emphysema and interstitial fibrosis of the lung; auditory disorders including hearing loss; and hematological disorders such as aplastic anemia and failed hematopoietic stem cell grafts.

### Uses of and iCM Factors

### Pharmaceutical Compositions

Since the gene expression changes associated with the EFT are often pan-cell and tissue type, and since cancer cells commonly display an embryonic (pre-fetal) pattern of gene expression, and as previously disclosed (see PCT International Application No. PCT/US2020/012640, titled “Compositions and Methods for Detecting Cardiotoxicity,” filed Jan. 7, 2020, contents of which are incorporated herein by reference) both segmental and global iCM factors have a widespread use in diverse cancer types. Non-limiting examples of such uses are discussed herein. In some embodiments, an iCM factor is used to mature cancer cells with an embryonic (pre-fetal) pattern of gene expression. Said cancer cells with an embryonic pattern of gene expression are found to be in all cancer types (i.e. are pan-cancer phenotypic alterations) and therefore, as previously disclosed (see PCT International Patent Application No. PCT/US2020/025512, titled “Induced tissue regeneration using extracellular vesicles,” filed Mar. 27, 2020; U.S. Provisional Application No. 63/274,731, titled “Methods for the Ex Vivo Induction of Tissue Regeneration in Microbiopsies,” filed Jan. 12, 2021; U.S. Provisional Application No. 63/274,731, titled “Use of Protocadherins in Methods of Diagnosing and Treating Cancer,” filed Nov. 2, 2021; U.S. Provisional Application No. 63/274,734, titled “Methods and Compositions Used to Modify Chromatin Architecture to Regulate Phenotype in Aging and Cancer,” filed Nov. 2, 2021; and U.S. Provisional Application No. 63/274,736, titled “Methods for the Temporal Regulation of Reprogramming Factors in Mammalian Cells,” filed Nov. 2, 2021, contents of each of which are incorporated herein by reference) include diverse carcinomas, adenocarcinomas, and sarcomas including but not limited to: acanthoma, acinar adenocarcinoma, acinic cell carcinoma, acrospiroma, acute eosinophilic leukemia, acute erythroid leukemia, acute lymphoblastic leukemia (ALL), acute megakaryoblastic leukemia, acute monocytic leukemia, acute myeloid leukemia (AML), acute promyelocytic leukemia, adamantinoma, adenoid cystic carcinoma, adenomatoid odontogenic tumor, adenosquamous carcinoma, adenosquamous lung carcinoma, adipose tissue neoplasm, adrenocortical carcinoma, adrenocortical carcinoma childhood, aggressive NK-cell leukemia, AIDS-related cancers, alveolar rhabdomyosarcoma, alveolar soft part sarcoma, ameloblastic fibroma, anal cancer, anaplastic carcinoma, anaplastic large-cell lymphoma, anaplastic thyroid cancer, angioimmunoblastic T-cell lymphoma, angiosarcoma, appendix cancer, attenuated familial adenomatous polyposis, atypical teratoid/rhabdoid tumor central nervous system childhood, B-cell chronic lymphocytic leukemia, B-cell lymphoma, bellini duct carcinoma, bile duct cancer, bile duct cancer—cholangiocarcinoma, bladder cancer, bladder cancer—small cell carcinoma, bladder cancer—transitional cell carcinoma, bladder cancer childhood, blastoma, bone cancer, bone cancer—osteosarcoma, brain stem glioma, brain tumors—other, brain tumor—glioblastoma multiforme, brain tumor—oligodendroglioma anaplastic, brain tumor—cerebellar astrocytoma (childhood & adult), brain tumor—cerebral astrocytoma/malignant glioma (childhood & adult), brain tumor—ependymoma, brain tumor—medulloblastoma, brain tumor—supratentorial primitive neuroectodermal tumors, brain tumor—visual pathway and hypothalamic glioma, brain and spinal cord tumors childhood, breast cancer, breast cancer ductal adenocarcinoma, breast cancer childhood, Brenner tumour, bronchial adenomas/carcinoids, bronchial tumors, bronchial tumors childhood, bronchioloalveolar carcinoma, Brown tumor, Burkitt lymphoma, carcinoid tumor, carcinoid tumor childhood, carcinoid tumor gastrointestinal, carcinoma of the penis, carcinosarcoma, cementoma, central nervous system cancer, cervical cancer—adenocarcinoma, cervical cancer—squamous cell, cervical cancer—neuroendocrine, carcinoma of the cervix, cervical cancer childhood, childhood cancers, childhood leukemia, cholangiocarcinoma, cholangiosarcoma, chondromyxoid fibroma, chondrosarcoma, chordoma, chorioadenoma destruens, chorioblastoma, choriocarcinoma, choroid plexus tumor, chorioepithelioma, clear cell adenocarcinoma, clear cell adenocarcinoma of the vagina, clear-cell ovarian carcinoma, clear-cell sarcoma of the kidney, colon cancer, colon cancer—adenocarcinoma, colorectal cancer, colorectal cancer childhood, comedocarcinoma, craniopharyngioma, craniopharyngioma childhood, cutaneous lymphoma, cystadenocarcinoma, degos disease, dermatofibrosarcoma protuberans, desmoplastic small round cell tumor, diffuse large B-cell lymphoma, digestive system neoplasm, diktyoma, ductal carcinoma in situ (DCIS), “ductal, lobular, and medullary neoplasms”, duodenal cancer, dysembryoplastic neuroepithelial tumour, dysgerminoma, ELM4-ALK positive lung cancer, embryoma, embryonal carcinoma, embryonal rhabdomyosarcoma, embryonal tumors central nervous system childhood, endocrine gland neoplasm, endodermal sinus tumor, endometrial cancer, endometrial-stromal sarcoma, endometrial-adenocarcinoma, endometrioid tumor, enteropathy-associated t-cell lymphoma, ependymoblastoma childhood, ependymoma childhood, epithelial-myoepithelial carcinoma of the lung, epithelioid sarcoma, epithelioma, esophageal cancer, esophageal cancer childhood, esthesioneuroblastoma childhood, ewing family of tumors, Ewing's sarcoma in the Ewing family of tumors, exocrine cancer, extracranial germ cell tumor childhood, extragonadal germ cell tumor, extrahepatic bile duct cancer, extramammary Paget's disease, eye cancer, “eye cancer, intraocular melanoma”, “eye cancer, retinoblastoma”, fallopian tube cancer, familial adenomatous polyposis, fetal adenocarcinoma, fibroepithelial neoplasms, fibrolamellar hepatocellular carcinoma, fibrosarcoma, fibrous tissue neoplasm, follicular lymphoma, follicular thyroid cancer, gcb diffuse large B-cell lymphoma (DLBCL), gallbladder cancer, ganglioglioma, ganglioneuroma, gardner's syndrome, gastric carcinoid, gastric (stomach) cancer, gastric (stomach) cancer—adenocarcinoma, gastric (stomach) cancer—adenocarcinoma of gastroesophageal junction, gastric (stomach) cancer childhood, gastric lymphoma, gastrinoma, gastrointestinal carcinoid tumor, gastrointestinal stromal tumors (GIST), germ cell tumor, extragonadal germ cell tumor, ovarian germ cell tumor, germinoma, gestational choriocarcinoma, gestational trophoblastic tumor, giant-cell fibroblastoma, giant-cell glioblastoma, giant-cell tumor of bone, gigantiform cementoma, glial tumor, gliomatosis cerebri, glioblastoma multiforme, glioma, glioma childhood visual pathway and hypothalamic, gliosarcoma, glucagonoma, goblet cell carcinoid, gonadoblastoma, granulosa cell tumour, gynandroblastoma, head and neck cancer, head and neck cancer childhood, heart cancer, hemangioblastoma, hemangiopericytoma, hemangiosarcoma, hematological malignancy, hepatic cancer—cholangiocarcinoma, hepatoblastoma, hepatocellular (liver) cancer, hepatosplenic t-cell lymphoma, hereditary breast-ovarian cancer syndrome, hereditary nonpolyposis colorectal cancer, histiocytic sarcoma, histiocytoma, hypopharyngeal cancer, inflammatory breast cancer, inflammatory myeloblastic tumor, intraductal carcinoma, intraductal papillary mucinous neoplasm, intraocular melanoma, intratubular germ cell neoplasia, invasive lobular carcinoma, islet cell carcinoma, islet cell tumors (endocrine pancreas), juvenile granulosa cell tumor, juvenile myelomonocytic leukemia, juxtaglomerular cell tumor, Kaposi sarcoma, kidney cancer childhood, Klatskin tumor, Krukenberg tumor, Langerhans cell histiocytosis, large-cell lung carcinoma with rhabdoid phenotype, laryngeal cancer, laryngeal cancer—squamous cell carcinoma, laryngeal cancer childhood, leiomyosarcoma, lentigo malignant melanoma, leptomeningeal cancer, leukemias, leydig cell tumour, chronic lymphocytic leukemia (CLL), chronic myelogenous leukemia (CML), hairy cell leukemia, linitis plastica, lip and oral cavity cancer, liposarcoma, liver cancer (primary), lobular carcinoma, lobular carcinoma in situ (LCIS), giant-cell carcinoma of the lung, large-cell lung carcinoma, large-cell lung carcinoma with rhabdoid phenotype, non-small cell lung cancer, lung—adenocarcinoma, lung—large cell_carcinoma, lung—small cell_carcinoma, lung—squamous cell_carcinoma, luteoma, lymphangioma, lymphangiosarcoma, lymphoepithelioma, lymphomas, lymphoma—extranodal marginal zone b-cell of lymphoid tissue, lymphoma—follicular cancer of lymphoid tissue, aids-related_lymphoma, cutaneous t-cell lymphoma, Hodgkin_lymphoma, non-Hodgkin lymphoma, primary central nervous system lymphoma (CNS), macroglobulinemia Waldenstrim, male breast cancer, malignant fibrous histiocytoma of bone and osteosarcoma, malignant peripheral nerve sheath tumor, malignant triton tumor, malt lymphoma, mammary ductal carcinoma, mantle cell lymphoma, marginal zone B-cell lymphoma, “Marcus Whittle, deadly disease”, mast cell leukemia, mediastinal germ cell tumor, mediastinal tumor, medullary carcinoma, medullary carcinoma of the breast, medullary thyroid cancer, medulloblastoma, medulloblastoma childhood, medulloepithelioma, medulloepithelioma childhood, melanoma, melanoma childhood, meningioma, Merkel cell carcinoma, mesenchymal chondrosarcoma, mesothelioma adult malignant, mesothelioma adult malignant—pleural mixed, mesothelioma childhood, metastatic breast cancer, metastatic squamous neck cancer with occult primary, metastatic tumor of jaws, metastatic urothelial carcinoma, mixed mullerian tumor, mouth cancer, mucinous cystadenocarcinoma of the lung, mucinous tumor, multiple endocrine neoplasia syndromes childhood, multiple endocrine neoplasia type 2b, multiple myeloma/plasma cell neoplasm, muscle tissue neoplasm, mycosis fungoides, myelodysplastic/myeloproliferative neoplasms, myelodysplastic syndromes, myeloid leukemia adult acute, myeloid leukemia childhood acute, myeloid sarcoma, chronic myeloproliferative disorders, myosarcoma, myxoid chondrosarcoma, myxoid liposarcoma, myxoma, myxosarcoma, nasal cavity and paranasal sinus cancer, nasopharyngeal angiofibroma, nasopharyngeal cancer, nasopharyngeal cancer childhood, nerve sheath tumor, nervous system neoplasm, neuroblastoma, neurocytoma, neurofibroma, neuroma, nipple adenoma, nodular lymphocyte predominant Hodgkin's lymphoma, nodular melanoma, odontogenic tumor, oncocytoma, optic nerve sheath meningioma, optic nerve tumor, oral cancer, oral cancer childhood, oropharyngeal cancer, oropharyngeal squamous cell carcinomas, osteolipochondroma, osteoma, osteosarcoma, ovarian cancer, ovarian cancer—adenocarcinoma of ovary serous, ovarian cancer childhood, ovarian cancer epithelial, ovarian cancer germ cell tumor, paget's disease of the breast, pancoast tumor, pancreatic cancer, pancreatic cancer childhood, pancreatic cancer—neuroendocrine, pancreatic cancer islet cell tumors, pancreatic—adenocarcinoma of pancreas ductal, pancreatic serous cystadenoma, papillary adenocarcinoma, papillary serous cystadenocarcinoma, papillary thyroid cancer, papillomatosis childhood, paraganglioma, parathyroid adenoma, parathyroid cancer, parathyroid neoplasm, PEComa, periampullary cancer, peritoneal mesothelioma, pharyngeal cancer, pheochromocytoma, pineal astrocytoma, pineal germinoma, pineal parenchymal tumors of intermediate differentiation childhood, pinealoblastoma, pineoblastoma and supratentorial primitive neuroectodermal tumors childhood, pineocytoma, pituicytoma, pituitary adenoma, pituitary tumor, plasma cell dyscrasia, plasma cell leukemia, plasma cell neoplasm/multiple myeloma, plasmacytoma, pleomorphic undifferentiated sarcoma, pleomorphic xanthoastrocytoma, pleuropulmonary blastoma, pleuropulmonary blastoma childhood, polyembryoma, posterior urethral cancer, precursor T-lymphoblastic lymphoma, primary peritoneal carcinoma, primitive neuroectodermal tumor, prostate cancer, prostate cancer—adenocarcinoma, pseudomyxoma peritonei, rectal cancer, rectal cancer—adenocarcinoma, renal cell carcinoma (kidney cancer), renal medullary carcinoma, renal pelvis and ureter transitional cell cancer, reninoma, respiratory tract neoplasm, retinoblastoma, rhabdomycin, rhabdomyosarcoma childhood, richter's transformation, salivary gland cancer, salivary gland cancer childhood, salivary gland-like carcinoma of the lung, salivary gland neoplasm, sacrococcygeal teratoma, sarcoma, sarcoma botryoides, sarcoma soft tissue, sarcomatoid carcinoma, schwannomatosis, sclerosing rhabdomyosarcoma, secondary neoplasm, seminoma, serous carcinoma, serous cystadenocarcinoma, serous tumour, sertoli cell tumour, sertoli-Leydig cell tumour, sex cord-gonadal stromal tumour, sgzary syndrome, signet ring cell carcinoma, skin cancer, skin cancer childhood, skin cancer—basal cell carcinoma, skin cancer—basal-like carcinoma, skin cancer—melanoma, small-cell carcinoma, small intestine cancer, “small-, round-, blue-cell tumour”, somatostatinoma, soot wart, spermatocytic seminoma, spinal tumor, spindle cell cancer, spindle cell rhabdomyosarcoma, splenic lymphoma with villous lymphocytes, splenic marginal zone lymphoma, squamous cell carcinoma, squamous neck cancer with occult primary metastatic, stewart_treves syndrome, stromal tumor, supratentorial primitive neuroectodermal tumors childhood, surface epithelial-stromal tumor, synovial sarcoma, t-cell lymphoma, t-lymphoblastic lymphoma, teratocarcinoma, testicular cancer, testicular cancer—seminoma, testicular cancer childhood, thecoma, throat cancer, “thymoma, childhood”, thymoma and thymic carcinoma, thymoma and thymic carcinoma childhood, thyroid cancer, thyroid cancer—follicular, thyroid cancer—papillary, thyroid cancer childhood, tonsil—carcinoma of tonsil squamous cell, trabecular cancer, tracheal tumor, transitional cell carcinoma, trophoblastic tumor gestational, tubulovillous adenoma, urachal cancer, ureteral cancer, ureteral neoplasm, urethral cancer, urogenital neoplasm, urothelial carcinoma, urothelial cell carcinoma, uterine cancer, uterine cancer endometrial, uterine clear cell carcinoma, uterine sarcoma, uterine serous carcinoma, uveal melanoma, vaginal cancer, vaginal cancer childhood, verrucous carcinoma, vestibular schwannoma, vipoma, visual pathway glioma, Von Hippel_Lindau disease, vulvar cancer, “Wilms tumor (kidney cancer), childhood.”

Other inventive methods comprise use of an iTM factor in the ex vivo production of living, functional tissues, organs, or cell-containing compositions with an adult-like phenotype to repair or replace a tissue or organ lost due to damage. While iTM reduces the regenerative potential of cells and tissues, in some applications the subject receiving such a transplant of cells and tissues is in need of immediate adult-like functionality. Alternatively, the cells or tissues may be partially matured by limiting the period of time said cells or tissues, such as microbiopsies, are exposed to said iTM factors. For example, cells or tissues removed from an individual (either the future recipient, an individual of the same species, or an individual of a different species) may be cultured in vitro, optionally with an matrix, scaffold (e.g., a three dimensional scaffold) or mold (e.g., comprising a biocompatible, optionally biodegradable, material, e.g., a polymer such as HyStem-C), and their development into a functional tissue or organ can be promoted by contacting an iTM factor. The scaffold, matrix, or mold may be composed at least in part of naturally occurring proteins such as collagen, hyaluronic acid, or alginate (or chemically modified derivatives of any of these), or synthetic polymers or copolymers of lactic acid, caprolactone, glycolic acid, etc., or self-assembling peptides, or decellularized matrices derived from tissues such as heart valves, intestinal mucosa, blood vessels, and trachea. In some embodiments, the scaffold comprises a hydrogel. The scaffold may, in certain embodiments, be coated or impregnated with an iTM factor, which may diffuse out from the scaffold over time. After production ex vivo, the tissue or organ is grafted into or onto a subject. For example, the tissue or organ can be implanted or, in the case of certain tissues such as skin, placed on a body surface. The tissue or organ may continue to develop in vivo. In some embodiments, the tissue or organ to be produced at least in part ex vivo is a bladder, blood vessel, bone, fascia, liver, muscle, skin patch, etc. Suitable scaffolds may, for example, mimic the extracellular matrix (ECM). Optionally, an iTM factor is administered to the subject prior to, during, and/or following grafting of the ex vivo generated tissue or organ. In some aspects, a biocompatible material is a material that is substantially non-toxic to cells in vitro at the concentration used or, in the case of a material that is administered to a living subject, is substantially nontoxic to the subject's cells in the quantities and at the location used and does not elicit or cause a significant deleterious or untoward effect on the subject, e.g., an immunological or inflammatory reaction, unacceptable scar tissue formation, etc. It will be understood that certain biocompatible materials may elicit such adverse reactions in a small percentage of subjects, typically less than about 5%, 1%, 0.5%, or 0.1%.

In some embodiments, a matrix or scaffold coated or impregnated with an iTM factor or combinations of factors including those capable of causing a global pattern of adult gene expression is implanted, optionally in combination with cells, into a subject in need of regeneration. The matrix or scaffold may be in the shape of a tissue or organ whose regeneration is desired. The cells may be stem cells of one or more type(s) that gives rise to such tissue or organ and/or of type(s) found in such tissue or organ.

In some embodiments, an iTR factor or combination of factors is administered directly to or near a site of tissue damage. “Directly to a site of tissue damage” encompasses injecting a compound or composition into a site of tissue damage or spreading, pouring, or otherwise directly contacting the site of tissue damage with the compound or composition. In some embodiments, administration is considered “near a site of tissue damage” if administration occurs within up to about 10 cm away from a visible or otherwise evident edge of a site of tissue damage or to a blood vessel (e.g., an artery) that is located at least in part within the damaged tissue or organ. Administration “near a site of tissue damage” is sometimes administration within a damaged organ, but at a location where damage is not evident. In some embodiments, an iTM factor is administered to enhance engraftment or of transplanted cells or tissues, since said iTM-treated cells have increased extracellular matrix production.

iTM and iCM factors such as exosomes derived from fetal or adult cells can be administered in physiological solutions such as saline, or slow-released in carboxymethyl hyaluronate crosslinked by PEGDA with carboxymethyl-modified gelatin (HyStem-C) to induce iTM or iCM.

In some embodiments a compound or composition is administered to a subject at least once within approximately 2, 4, 8, 12, 24, 48, 72, or 96 hours after a subject has suffered tissue damage (e.g., an injury or an acute disease-related event such as a myocardial infarction or stroke) and, optionally, at least once thereafter. In some embodiments a compound or composition is administered to a subject at least once within approximately 1-2 weeks, 2-6 weeks, or 6-12 weeks, after a subject has suffered tissue damage and, optionally, at least once thereafter.

In some aspects, an iTM factor is used to enhance regeneration of connective tissue such as tendon and ligament tissue.

iTM and iCM factors may be tested in a variety of animal models of regeneration. In one aspect, a modulator of iTM or iCM is tested in murine species. For example, human cancer cell xenografts in mice, rats, or other mammalian model systems may be used to test the applications of iCM for a variety of cancer cell types for propensity for tumor growth, survival over time, resistance to radio- and chemotherapy, and cancer cell metastasis.

The compounds and compositions disclosed herein and/or identified using a method and/or assay system described herein may be administered by any suitable means such as orally, intranasally, subcutaneously, intramuscularly, intravenously, intra-arterially, parenterally, intraperitoneally, intrathecally, intratracheally, ocularly, sublingually, vaginally, rectally, dermally, or by inhalation, e.g., as an aerosol. The particular mode selected will depend, of course, upon the particular compound selected, the particular condition being treated and the dosage required for therapeutic efficacy. The methods of this invention, generally speaking, may be practiced using any mode of administration that is medically or veterinarily acceptable, meaning any mode that produces acceptable levels of efficacy without causing clinically unacceptable (e.g., medically or veterinarily unacceptable) adverse effects. Suitable preparations, e.g., substantially pure preparations, of one or more compound(s) may be combined with one or more pharmaceutically acceptable carriers or excipients, etc., to produce an appropriate pharmaceutical composition suitable for administration to a subject. Such pharmaceutically acceptable compositions are an aspect of the invention. The term “pharmaceutically acceptable carrier or excipient” refers to a carrier (which term encompasses carriers, media, diluents, solvents, vehicles, etc.) or excipient which does not significantly interfere with the biological activity or effectiveness of the active ingredient(s) of a composition and which is not excessively toxic to the host at the concentrations at which it is used or administered. Other pharmaceutically acceptable ingredients can be present in the composition as well. Suitable substances and their use for the formulation of pharmaceutically active compounds are well-known in the art (see, for example, “Remington's Pharmaceutical Sciences”, E. W. Martin, 19th Ed., 1995, Mack Publishing Co.: Easton, Pa., and more recent editions or versions thereof, such as Remington: The Science and Practice of Pharmacy. 21st Edition. Philadelphia, Pa. Lippincott Williams & Wilkins, 2005, for additional discussion of pharmaceutically acceptable substances and methods of preparing pharmaceutical compositions of various types). Furthermore, compounds and compositions of the invention may be used in combination with any compound or composition used in the art for treatment of a particular disease or condition of interest.

A pharmaceutical composition is typically formulated to be compatible with its intended route of administration. For example, preparations for parenteral administration include sterile aqueous or non-aqueous solutions, suspensions, and emulsions. Aqueous carriers include water, alcoholic/aqueous solutions, emulsions or suspensions, including saline and buffered media, e.g., sodium chloride solution, Ringer's dextrose, dextrose and sodium chloride, lactated Ringer's. Examples of non-aqueous solvents are propylene glycol, polyethylene glycol, vegetable oils such as olive oil, and injectable organic esters such as ethyl oleate. fixed oils, polyethylene glycols, glycerine, propylene glycol or other synthetic solvents; preservatives, e.g., antibacterial agents such as benzyl alcohol or methyl parabens; antioxidants such as ascorbic acid or sodium bisulfite; chelating agents such as ethylenediaminetetraacetic acid; buffers such as acetates, citrates or phosphates, and agents for the adjustment of tonicity such as sodium chloride or dextrose. pH can be adjusted with acids or bases, such as hydrochloric acid or sodium hydroxide. Such parenteral preparations can be enclosed in ampoules, disposable syringes or multiple dose vials made of glass or plastic.

For oral administration, compounds can be formulated readily by combining the active compounds with pharmaceutically acceptable carriers well known in the art. Such carriers enable the compounds of the invention to be formulated as tablets, pills, dragees, capsules, liquids, gels, syrups, slurries, suspensions and the like. Suitable excipients for oral dosage forms are, e.g., fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl cellulose, sodium carboxymethylcellulose, and/or polyvinylpyrrolidone (PVP).

For administration by inhalation, inventive compositions may be delivered in the form of an aerosol spray from a pressured container or dispenser which contains a suitable propellant, e.g., a gas such as carbon dioxide, a fluorocarbon, or a nebulizer. Liquid or dry aerosol (e.g., dry powders, large porous particles, etc.) can be used. The present invention also contemplates delivery of compositions using a nasal spray or other forms of nasal administration.

For topical applications, pharmaceutical compositions may be formulated in a suitable ointment, lotion, gel, or cream containing the active components suspended or dissolved in one or more pharmaceutically acceptable carriers suitable for use in such composition.

For local delivery to the eye, the pharmaceutically acceptable compositions may be formulated as solutions or micronized suspensions in isotonic, pH adjusted sterile saline, e.g., for use in eye drops, or in an ointment, or for intra-ocularly administration, e.g., by injection.

Pharmaceutical compositions may be formulated for transmucosal or transdermal delivery. For transmucosal or transdermal administration, penetrants appropriate to the barrier to be permeated may be used in the formulation. Such penetrants are generally known in the art. Inventive pharmaceutical compositions may be formulated as suppositories (e.g., with conventional suppository bases such as cocoa butter and other glycerides) or as retention enemas for rectal delivery.

In some embodiments, a composition includes one or more agents intended to protect the active agent(s) against rapid elimination from the body, such as a controlled release formulation, implants, microencapsulated delivery system, etc. Compositions may incorporate agents to improve stability (e.g., in the gastrointestinal tract or bloodstream) and/or to enhance absorption. Compounds may be encapsulated or incorporated into particles, e.g., microparticles or nanoparticles. Biodegradable, biocompatible polymers can be used, such as ethylene vinyl acetate, polyanhydrides, polyglycolic acid, PLGA, collagen, polyorthoesters, polyethers, and polylactic acid. Methods for preparation of such formulations will be apparent to those skilled in the art. For example, and without limitation, a number of particle, lipid, and/or polymer-based delivery systems are known in the art for delivery of siRNA. The invention contemplates use of such compositions. Liposomes or other lipid-based particles can also be used as pharmaceutically acceptable carriers.

Pharmaceutical compositions and compounds for use in such compositions may be manufactured under conditions that meet standards, criteria, or guidelines prescribed by a regulatory agency. For example, such compositions and compounds may be manufactured according to Good Manufacturing Practices (GMP) and/or subjected to quality control procedures appropriate for pharmaceutical agents to be administered to humans and can be provided with a label approved by a government regulatory agency responsible for regulating pharmaceutical, surgical, or other therapeutically useful products.

Pharmaceutical compositions of the invention, when administered to a subject for treatment purposes, are preferably administered for a time and in an amount sufficient to treat the disease or condition for which they are administered. Therapeutic efficacy and toxicity of active agents can be assessed by standard pharmaceutical procedures in cell cultures or experimental animals. The data obtained from cell culture assays and animal studies can be used in formulating a range of dosages suitable for use in humans or other subjects. Different doses for human administration can be further tested in clinical trials in humans as known in the art. The dose used may be the maximum tolerated dose or a lower dose. A therapeutically effective dose of an active agent in a pharmaceutical composition may be within a range of about 0.001 mg/kg to about 100 mg/kg body weight, about 0.01 to about 25 mg/kg body weight, about 0.1 to about 20 mg/kg body weight, about 1 to about 10 mg/kg. Other exemplary doses include, for example, about 1 g/kg to about 500 mg/kg, about 100 g/kg to about 5 mg/kg. In some embodiments a single dose is administered while in other embodiments multiple doses are administered. Those of ordinary skill in the art will appreciate that appropriate doses in any particular circumstance depend upon the potency of the agent(s) utilized, and may optionally be tailored to the particular recipient. The specific dose level for a subject may depend upon a variety of factors including the activity of the specific agent(s) employed, the particular disease or condition and its severity, the age, body weight, general health of the subject, etc. It may be desirable to formulate pharmaceutical compositions, particularly those for oral or parenteral compositions, in unit dosage form for ease of administration and uniformity of dosage. Unit dosage form, as that term is used herein, refers to physically discrete units suited as unitary dosages for the subject to be treated; each unit containing a predetermined quantity of active agent(s) calculated to produce the desired therapeutic effect in association with an appropriate pharmaceutically acceptable carrier. It will be understood that a therapeutic regimen may include administration of multiple doses, e.g., unit dosage forms, over a period of time, which can extend over days, weeks, months, or years. A subject may receive one or more doses a day, or may receive doses every other day or less frequently, within a treatment period. For example, administration may be biweekly, weekly, etc. Administration may continue, for example, until appropriate structure and/or function of a tissue or organ has been at least partially restored and/or until continued administration of the compound does not appear to promote further regeneration or improvement. In some embodiments, a subject administers one or more doses of a composition of the invention to him or herself.

In some embodiments, two or more compounds or compositions are administered in combination, e.g., for purposes of enhancing regeneration. Compounds or compositions administered in combination may be administered together in the same composition, or separately. In some embodiments, administration “in combination” means, with respect to administration of first and second compounds or compositions, administration performed such that (i) a dose of the second compound is administered before more than 90% of the most recently administered dose of the first agent has been metabolized to an inactive form or excreted from the body; or (ii) doses of the first and second compound are administered within 48, 72, 96, 120, or 168 hours of each other, or (iii) the agents are administered during overlapping time periods (e.g., by continuous or intermittent infusion); or (iv) any combination of the foregoing. In some embodiments, two or more iTR factors, or vectors expressing the catalytic component of telomerase and an iTR factor, are administered. In some embodiments an iTR factor is administered in combination with a combination with one or more growth factors, growth factor receptor ligands (e.g., agonists), hormones (e.g., steroid or peptide hormones), or signaling molecules, useful to promote regeneration and polarity. Of particular utility are organizing center molecules useful in organizing regeneration competent cells such as those produced using the methods of the present invention. In some embodiments, a growth factor is an epidermal growth factor family member (e.g., EGF, a neuregulin), a fibroblast growth factor (e.g., any of FGF1-FGF23), a hepatocyte growth factor (HGF), a nerve growth factor, a bone morphogenetic protein (e.g., any of BMP1-BMP7), a vascular endothelial growth factor (VEGF), a wnt ligand, a wnt antagonist, retinoic acid, NOTUM, follistatin, sonic hedgehog, or other organizing center factors.

Those skilled in the art will recognize, or be able to ascertain using no more than routine experimentation, many equivalents to the specific embodiments of the invention described herein. The scope of the present invention is not intended to be limited to the Description or the details set forth therein. Articles such as “a”, “an” and “the” may mean one or more than one unless indicated to the contrary or otherwise evident from the context. Certain of the inventive methods are often practiced using populations of cells, e.g., in vitro or in vivo. Thus references to “a cell” should be understood as including embodiments in which the cell is a member of a population of cells, e.g., a population comprising or consisting of cells that are substantially genetically identical. However, the invention encompasses embodiments in which inventive methods is/are applied to an individual cell. Thus, references to “cells” should be understood as including embodiments applicable to individual cells within a population of cells and embodiments applicable to individual isolated cells.

Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention also includes embodiments in which more than one, or all of the group members are present in, employed in, or otherwise relevant to a given product or process. It is contemplated that all embodiments described herein are applicable to all different aspects of the invention. It is also contemplated that any of the embodiments can be freely combined with one or more other such embodiments whenever appropriate. Furthermore, it is to be understood that the invention encompasses all variations, combinations, and permutations in which one or more limitations, elements, clauses, descriptive terms, etc., from one or more of the claims (whether original or subsequently added claims) is introduced into another claim (whether original or subsequently added). For example, any claim that is dependent on another claim can be modified to include one or more elements or limitations found in any other claim that is dependent on the same base claim, and any claim that refers to an element present in a different claim can be modified to include one or more elements or limitations found in any other claim that is dependent on the same base claim as such claim. Furthermore, where the claims recite a composition, the invention provides methods of making the composition, e.g., according to methods disclosed herein, and methods of using the composition, e.g., for purposes disclosed herein. Where the claims recite a method, the invention provides compositions suitable for performing the method, and methods of making the composition. Also, where the claims recite a method of making a composition, the invention provides compositions made according to the inventive methods and methods of using the composition, unless otherwise indicated or unless one of ordinary skill in the art would recognize that a contradiction or inconsistency would arise. Where elements are presented as lists, e.g., in Markush group format, each subgroup of the elements is also disclosed, and any element(s) can be removed from the group. For purposes of conciseness only some of these embodiments have been specifically recited herein, but the invention includes all such embodiments. It should also be understood that, in general, where the invention, or aspects of the invention, is/are referred to as comprising particular elements, features, etc., certain embodiments of the invention or aspects of the invention consist, or consist essentially of, such elements, features, etc.

Where numerical ranges are mentioned herein, the invention includes embodiments in which the endpoints are included, embodiments in which both endpoints are excluded, and embodiments in which one endpoint is included and the other is excluded. It should be assumed that both endpoints are included unless indicated otherwise. Furthermore, unless otherwise indicated or otherwise evident from the context and understanding of one of ordinary skill in the art, values that are expressed as ranges can assume any specific value or subrange within the stated ranges in different embodiments of the invention, to the tenth of the unit of the lower limit of the range, unless the context clearly dictates otherwise. Where phrases such as “less than X”, “greater than X”, or “at least X” is used (where X is a number or percentage), it should be understood that any reasonable value can be selected as the lower or upper limit of the range. It is also understood that where a list of numerical values is stated herein (whether or not prefaced by “at least”), the invention includes embodiments that relate to any intervening value or range defined by any two values in the list, and that the lowest value may be taken as a minimum and the greatest value may be taken as a maximum. Furthermore, where a list of numbers, e.g., percentages, is prefaced by “at least”, the term applies to each number in the list. For any embodiment of the invention in which a numerical value is prefaced by “about” or “approximately”, the invention includes an embodiment in which the exact value is recited. For any embodiment of the invention in which a numerical value is not prefaced by “about” or “approximately”, the invention includes an embodiment in which the value is prefaced by “about” or “approximately”. “Approximately” or “about” generally includes numbers that fall within a range of 1% or in some embodiments 5% or in some embodiments 10% of a number in either direction (greater than or less than the number) unless otherwise stated or otherwise evident from the context (e.g., where such number would impermissibly exceed 100% of a possible value). A “composition” as used herein, can include one or more than one component unless otherwise indicated. For example, a “composition comprising an activator or a TR activator” can consist or consist essentially of an activator of a TR activator or can contain one or more additional components. It should be understood that, unless otherwise indicated, an inhibitor or a TR inhibitor (or other compound referred to herein) in any embodiment of the invention may be used or administered in a composition that comprises one or more additional components including the presence of an activator of a TR activator.

### EXAMPLES

### Example 1 COX7A1 as a Segmental Regulator of Both iTM and iCM

As previously disclosed (see PCT International Patent Application No. PCT/US14/40601, titled “Compositions and Methods for Induced Tissue Regeneration in Mammalian Species,” filed Jun. 3, 2014; and PCT International Patent Application No. PCT/US2017/036452, titled “Improved Methods for Detecting and Modulating the Embryonic-Fetal Transition in Mammalian Cells,” filed Jun. 7, 2017, contents of each of which are incorporated herein by reference) COX7A1 is not expressed in PSCs or in PSC-derived embryonic progenitor cell types of some 200-fold diversity including differentiated progeny of said PSC-derived progenitor cell lines. The applicants teach that this lack of maturation reflects the lack of normal endocrine signaling cascades that can be introduced artificially to induce iTM. The applicants furthermore teach that similarly diverse carcinoma, adenocarcinoma, and sarcoma cell types display an embryonic (pre-fetal) pattern of gene expression reflecting an escape from the normal tumor suppression afforded by fetal and adult patterns of gene expression. Furthermore, thee applicants teach that contrary to the teachings in the art, cancer stem cells (CSCs) are not more developmentally immature than other cancer cells, but are, in reality, largely adult-like in gene expression patterns. Exceptions are the expression of telomerase (TERT) in the majority of cancer types that is normally characteristic of PSCs, but not PSC-derived cells. The applicants further teach that CSCs express a mesenchymal phenotype as a result of a fetal or adult-like pattern of gene expression that promotes thee mesenchymal phenotype (EMT).

As an example of segmental iTM and segmental iCM, COX7A1 and eGFP was inserted into lentivirus constructs and non-cancerous pluripotent stem cell-derived cells and cancer cells with an embryonic pattern of gene expression were infected with each construct. The embryonic (pre-fetal) pluripotent cell-derived clonal embryonic progenitor cells capable of chondrogenic differentiation used are designated 4D20.8, and they display an embryonic (pre-fetal) pattern of gene expression (for example, not expressing the post EFT gene expression markers COX7A1 or PCDHGA12) even when differentiated into cartilage (FIGS. 2A-2D). To determine the efficacy of COX7A1 as a segmental factor for iCM, the fibrosarcoma cancer cell line HT1080 was utilized that expresses an embryonic pattern of gene expression (for example, not expressing the post EFT gene expression markers COX7A1 or PCDHGA12) was used to test iCM. The HT1080 fibrosarcoma and 4D20.8 hES-derived clonal progenitor line was transduced using 1) Genecopoeia LPP-C0223Lv205-200 COX7A1 (NM_001864.3) purified lentiviral particles containing CMV promotor and IRES2-eGFP-IRESpuromycin Tag and 2) Gencopoeia LPP-EGFP-Lv105-100-C eGFP lentifect purified lentiviral particles containing CMV promotor and IR ES2-eGFP-IRES-puromycin Tag, Conc 10⁸ TU/ml MOI=10.

The protocol included the steps of: 1) A polybrene sensitivity (0, 4 and 8 μg/ml) test and puromycin dose response (0, 2, 4, 6, 8, 10 g/mil) kill curve analysis on both lines was first performed in 96 well plate to determine optimal concentrations of exposure during transduction and clone selection respectively. 2) The cells were harvested in log phase growth and plated in 24 well plates at multiple cell densities in respective growth mediums (2.0 mL final volume) to determine optimal seeding number so that the wells are ˜80% confluent 24 hours later. 3) Then, once seeding density was determined for each cell line, 24 well plates were set up in triplicate (media control, Polybrene control and lentivirus of interest). 4) Cells were incubated at 37° C., 5% CO₂ for −24 hours. 5) Culture media was replaced with fresh polybrene containing media and viral particles added to appropriate wells. Cells were gently swirled and let stand at room temperature for 30 min. 6) Plates were centrifuged at 800×g for 30 minutes at 32° C. with slow acceleration and slow deceleration. Then the plates are removed carefully without disturbing the cells. 7) The cells were incubated at 37° C., 5% CO₂ for 24-48 hr in virus containing media. Then the viral containing media was removed and cells allowed to incubate for an additional two days in fresh culture without selection agent.

### Clonal Selection and Vector Expression Determination

After two days, the cells were incubated at 37° C., 5% CO₂ for an additional 4 days in fresh culture medium containing selection agent. The cells with GFP+ (top 10%) were selected using a single cell sorter (FACS Melody Cell Sorter, BD) into 96-well plates. The selected clones were expanded into 6 well plates and 10 cm dishes in medium containing selection agent and incubated at 37° C., 5% CO₂ until confluent. The top expressing clones from transduced lines were cryopreserved for later use.

### Prep for RNA for RNAseq

Following thawing the four lines (HT1080 with eGFP or COX7A1, and 4D20.8 with eGFP or COX7A1), they were placed in T-25 flasks and cultured and expanded in their respective growth medium for one week. Then 100,000 cells were seeded on 0.1% gelatin coated cultureware in 6 well plates in their respective medium (4D20.8, DMEM 20% FBS), and HT1080, DMEM 10% FBS). They were placed in a humidified incubator with 10% CO₂ and 5% 02 at 37° C. At confluence were fasted in DMEM 0.5% FBS for five days (three days then refed fast medium for 2 more days). They were then lysed with 350 ul RLT lysis buffer (Qiagen) and after using a cell scraper the material is removed and placed in RNase DNase free microfuge tubes. RNA was prepared using Qiagen RNAeasy mini kits (Cat #74104) following manufacturer's directions.

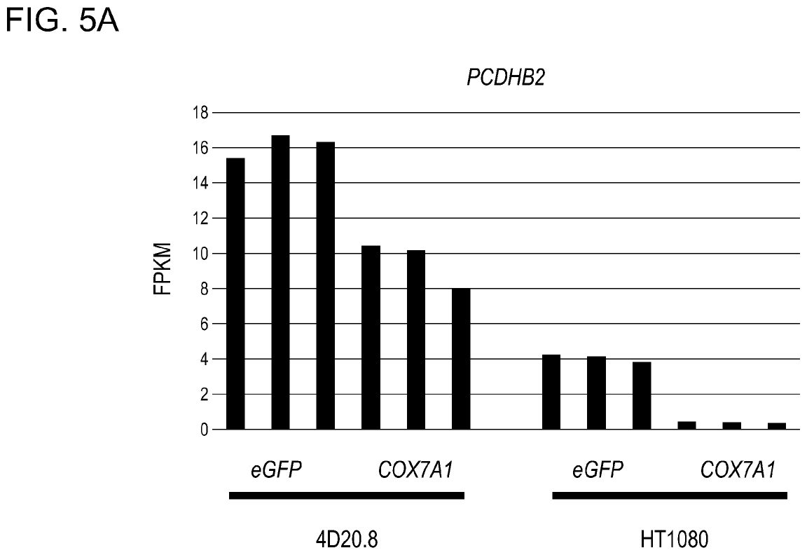

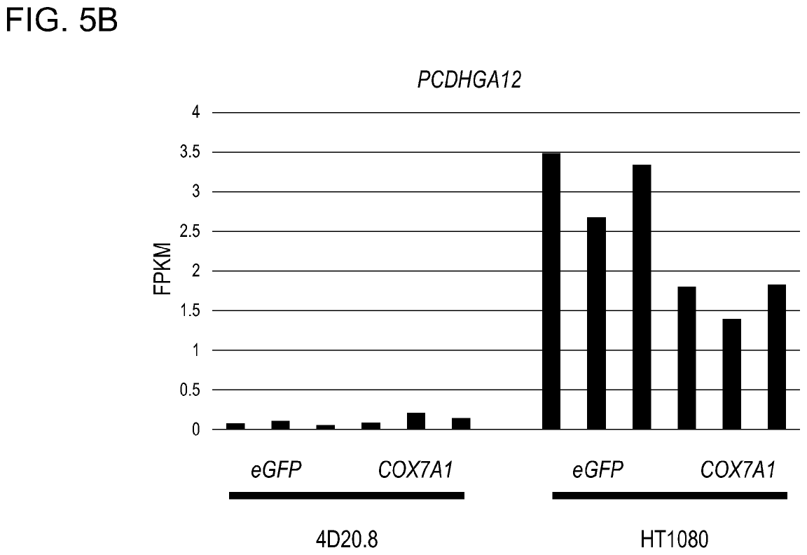

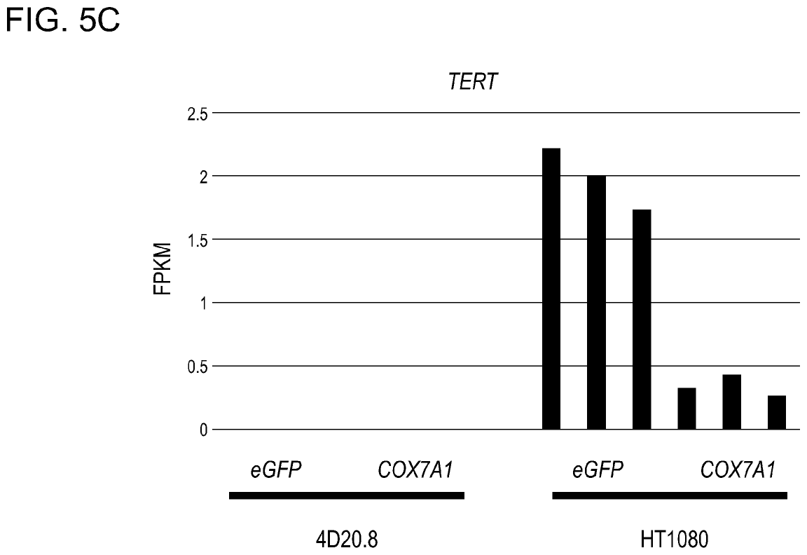

As shown in FIG. 5A, the exogenous expression of COX7A1 (but not the eGFP control) reduced the expression of the embryonic marker PCDHB2 in the embryonic progenitor cell line 4D20.8 as well as the HT1080 cells in three separate experiments. However, as shown in FIG. 5B, the adult-onset gene PCDHGA12 was not affected. Therefore, COX7A1 is a “segmental” regulator of both iTM and iCM. Surprisingly, however, COX7A1 reproducibly inhibited transcript levels of TERT (FIG. 5C). Since TERT is responsible for the immortal proliferation of ˜90% of human cancer cells, this unanticipated effect of COX7A1 as a segmental regulator of iCM may have additional benefits in limiting the replicative lifespan of cancer cells.

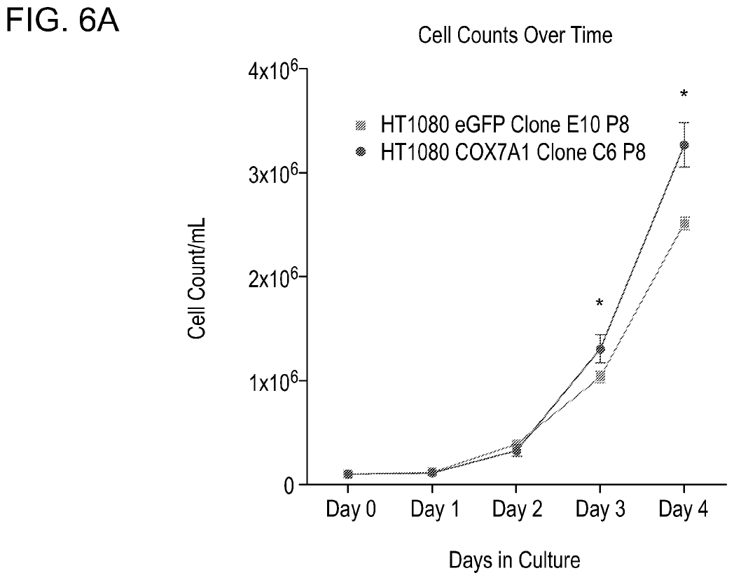

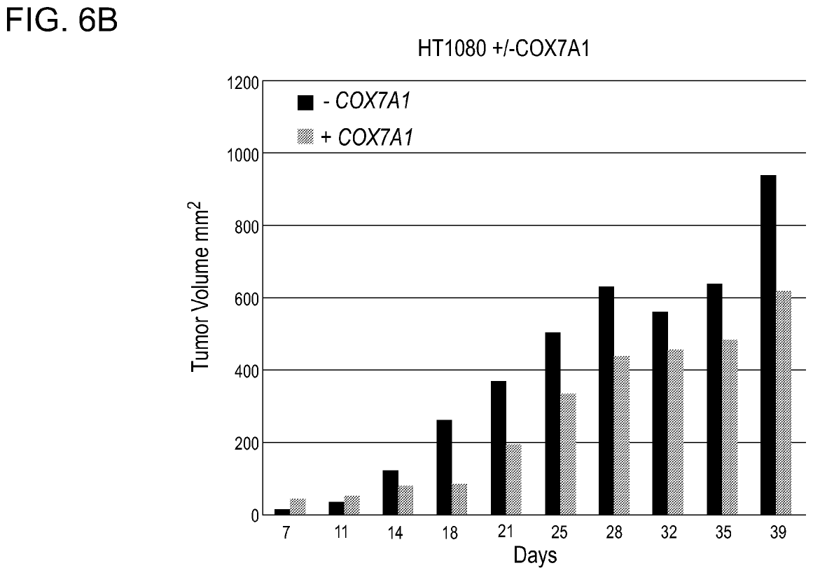

As shown in FIG. 6A, the exogenous administration of COX7A1 also significantly slowed the growth of HT1080 cells both in vitro and in a NOD/SCID xenograft tumor growth model in vivo in mice (FIG. 6B).

Briefly, CR female NCr nu/nu mice were injected with 1×10⁷ HT1080-AXI tumor cells in 0% Matrigel sc in the flank. Cell Injection Volume (e.g., HT1080-AXI tumor cells or HT1080-COX-AXI cells) was 0.1 mL/mouse. Cell lines were MAP tested. Mice were 6 to 9 weeks old at the start of the assay. Weight measurements and caliper measurements of the tumor size started at day 7. Any individual animal with a single observation of >than 30% body weight loss or three consecutive measurements of >25% body weight loss was euthanized. Dosing was stopped for any group with a mean body weight loss of >20% or >10% mortality. The group was not euthanized and recovery was allowed. Within a group with >20% weight loss, individuals hitting the individual body weight loss endpoint will be euthanized. If the group treatment related body weight loss was recovered to within 10% of the original weights, dosing resumed at a lower dose or less frequent dosing schedule. Exceptions to non-treatment body weight % recovery were allowed on a case-by-case basis.

The endpoint of the experiment was a tumor volume of 1000 mm³ or 60 days, whichever came first. Responders were followed longer. Animals were randomized into treatment groups based on Day 1 bodyweight. The study started at the day of implant (Day 1).

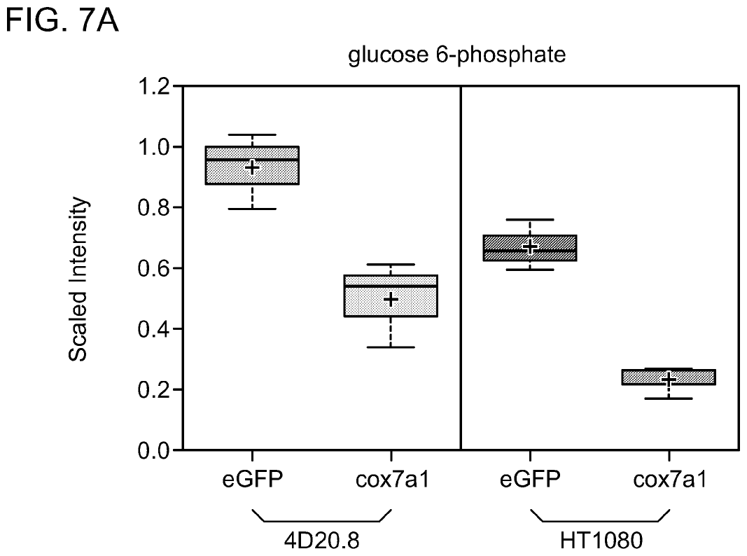

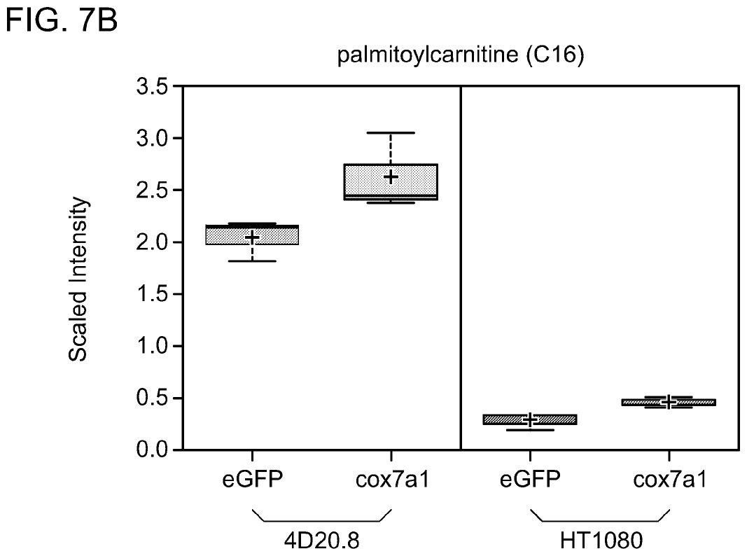

To further examine the segmental iTM and iCM effects on metabolism, COX7A1 expressing and eGFP controls for both 4D20.8 and the cancer cell line HT1080 were cultured in vitro and then extracted for metabolic analysis by mass spectrometry. As shown in FIG. 7A, COX7A1 expression broadly altered metabolic pathways in both normal embryonic 4D20.8 cells as well as embryonic-like HT1080 cells compared to eGFP controls. COX7A1 diminished glucose oxidation in both cell types such as glucose 6-phosphate as well as other glycolytic intermediates, and elevated fatty acid oxidation with significantly elevated long-chain fatty acids (FIG. 7B), consistent with elevated Randle cycle as a homeostatic mechanism that mediates reciprocal control of glucose and fatty acid oxidation (PMID: 13990765).

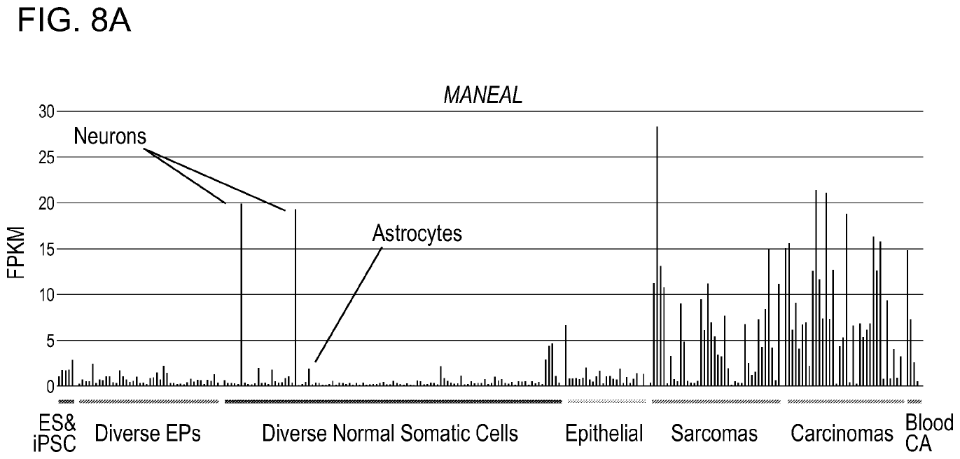

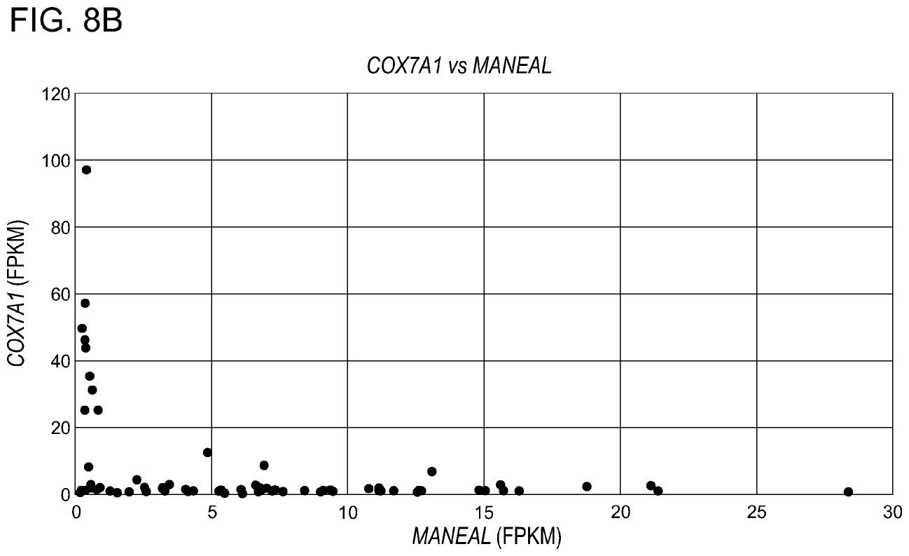

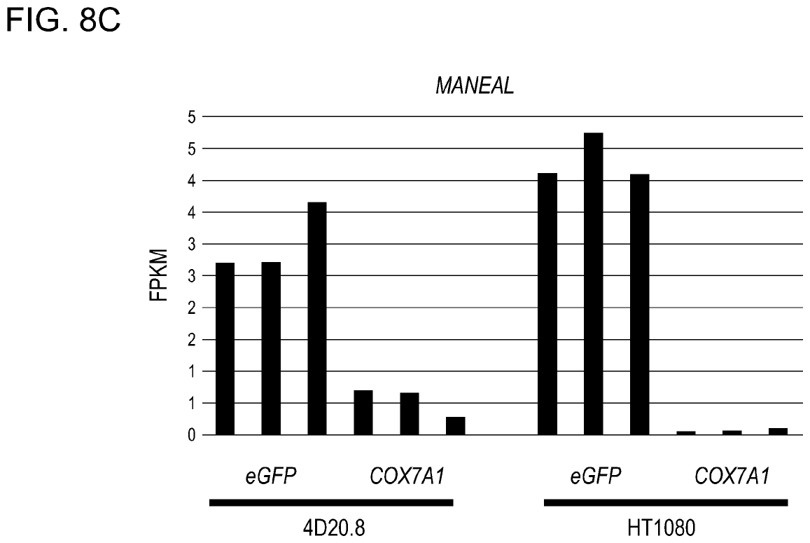

Surprisingly, the gene encoding Mannosidase, Endo-Alpha-Like (MANEAL) than is down-regulated in diverse adult cell types but markedly up-regulated in cancer cells (FIG. 8A), was markedly down-regulated in both normal embryonic progenitors (4D20.8) and in HT1080 when COX7A1 was expressed compared to eGFP controls (FIG. 8B). Further supporting the role of COX7A1 in regulating MANEAL expression, there was a marked inverse correlation of COX7A1 expression with MANEAL in diverse cancer cell lines (FIG. 8C). This reflects an increase in protein folding repair during the embryonic stages of development and cancer, and evidence for the benefit of iTR in the treatment of protein folding disorders such as Alzheimer s disease.

### Example 2. T3 as a Global iTM Modulator

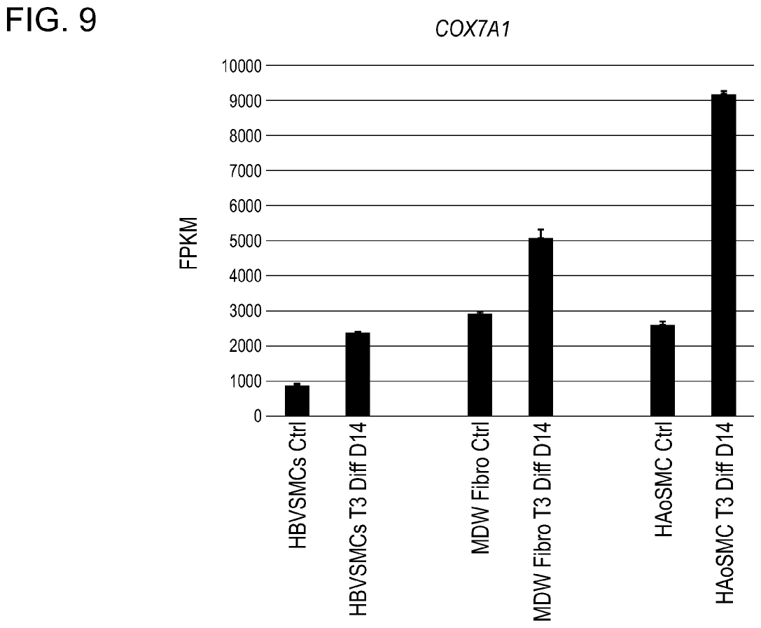

Diverse fetal or adult derived human somatic cells (Human Brain Vascular Smooth Muscle Cells (HBVSC), dermal fibroblasts (MDW), and Human Aortic Smooth Muscle Cells (HAoSMCs) and a PSC-derived clonal embryonic progenitor cell line NP1-2-26 were treated for 14 days with 2.0 nM thyroid hormone (T3) and the transcriptomic expression of the fetal and adult marker COX7A1 that progressively increases during development through adulthood as well as other adult markers were determined to determine whether the hormone T3 (which is the most active form of thyroid hormone) functioned as a global iTM factor. As shown in FIG. 9, the cells showed a marked increase in COX7A1 expression consistent with T3 being an iTM factor. In the embryonic progenitor line NP1-2-26, in addition to an induction of COX7A1, other adult onset genes were up-regulated including NKAPL, LMNA, COMT, as well as markers of CSCs such as ALCAM and EMT markers such as COL1A1. Therefore, T3 functions as a global iTM and iCM modulator.

What is claimed is:

1. A method of maturing a mammalian cell that expresses an embryonic (pre-fetal) pattern of gene expression into a cell that expresses markers of fetal or adult cells, said method comprising administering one or more endocrine factors to said cells.

2. The method of claim 1, wherein the endocrine factors are selected from the group consisting of T3, T4, cortisol, dexamethasone, proopiomelanocortin (POMC) and POMC derivative peptides, a MAP kinase activator, FGF7, IGF2, and Growth hormone (GH), wherein the POMC derivatives are N-terminal peptide of proopiomelanocortin (NPP), alpha melanotropin (αMSH), beta-melanotropin (βMSH), delta-melanocyte-stimulating hormone (δMSH), epsilson-melanocyte-stimulating hormone (εMSH), corticotropin, beta-lipotropin, gamma lipotropin, beta-endorphin, and met-enkephalin, wherein the MAP kinase is an ERK1/2 activator, e.g., baicalein or baicalin, and lamin A.

3. The method of claim 1, wherein the endocrine factors are 1) T3 or T4; and 2) one or more of cortisol, dexamethasone, proopiomelanocortin (POMC) and POMC derivative peptides, a MAP kinase activator, FGF7, IGF2, and Growth hormone (GH), wherein the POMC derivatives are N-terminal peptide of proopiomelanocortin (NPP), alpha melanotropin (αMSH), beta-melanotropin (βMSH), delta-melanocyte-stimulating hormone (δMSH), epsilson-melanocyte-stimulating hormone (εMSH), corticotropin, beta-lipotropin, gamma lipotropin, beta-endorphin, and met-enkephalin, wherein the MAP kinase is an ERK1/2 activator, e.g., baicalein or baicalin, and lamin A.

4. The method of claim 2 or claim 3, wherein the endocrine factors are 1) T3 or T4; and 2) one or more of cortisol, dexamethasone, FGF7, IGF2, and Growth hormone (GH).

5. The method of claim 2 or claim 3, wherein the endocrine factors are 1) T3 or T4; and 2) ERK1/2 activator.

6. The method of claim 2 or claim 3, wherein the endocrine factors are 1) T3 or T4; and 2) FGF7.

7. The method of claim 2 or claim 3, wherein the endocrine factors are 1) T3 or T4; and 2) one or more of proopiomelanocortin (POMC) and POMC derivative peptides, wherein the POMC derivatives are N-terminal peptide of proopiomelanocortin (NPP), alpha melanotropin (αMSH), beta-melanotropin (βMSH), delta-melanocyte-stimulating hormone (δMSH), epsilson-melanocyte-stimulating hormone (εMSH), corticotropin, beta-lipotropin, gamma lipotropin, beta-endorphin, and met-enkephalin.

8. The method of any one of claims 2-7, further comprising administering one or more induced tissue maturation (iTM) factors.

9. The method of claim 8, wherein the one or more iTM factors are one or more tissue regeneration (TR) inhibitory genes, one or more inhibitors of one or more tissue regeneration (TR) activator genes, or a combination thereof.

10. The method of claim 9, wherein the one or more TR inhibitory genes are selected from the group consisting of ACAT2, C18orf56, CAT, COMT, COX7A1, DYNLT3, ELOVL6, FDPS, IAH1, INSIG1, LOC205251, MAOA, RPS7, SHMT1, TRIM4, TSPYL5, or ZNF280D.

11. The method of claim 9, wherein the one or more TR inhibitory genes are selected from the group consisting of ADIRF, C10orf11, CAT, CCDC144B, COMT, COX7A1, KRBOX1, LINC00654, LINC00839, LINC01116, MEG3, MIR4458HG, PCDHGA12, PCDHGB3, PCDHGB5, PLPP7 (PPAPDC3), POMC, PRR34, PRSS3, PTCHD3, PTCHD3P1, SPESP1, TRIM4, USP32P1, ZNF300P1, or ZNF572.

12. The method of claim 9, wherein the one or more TR inhibitory genes are selected from the group consisting of ALS2CR11, C2CD6, ANKRD7, ANKRD65, BACE2, BHMT2, C22orf26, CADPS2, CALHM2, CCDC36, CCDC89, CCDC125, CCDC144B, CLDN11, CTSF, DDX43, DNAJC15, EGFLAM, ESPNL, FAM24B, FGF7, FKBP9L, FLG-AS1, FRG1B, GPAT2, GYPE, HENMT1, HIST2H2BA, IRAK4, LINC00865, LOC283788, LOC100233156, LRRK2, MAP10, MEG8, MEG9, MIRLET7HG, NKAPL, PAX8-AS1, PRPH2, PRR34-AS1, RP5-1043L13.1, RP11-134021.1, SVIL-AS1, TEKT4P2, ZNF578, ZNF585B, ZNF736, or ZNF790-AS1.

13. The method of claim 9, wherein the TR inhibitory gene is FGF7.

14. The method of any one of claims 9-13, wherein the one of more TR activator genes are selected from the group consisting of AFF3, CBCAQH03 5, DLX1, DRD1IP, F2RL2, FOXD1, LOC728755, LOC791120, MN1, OXTR, PCDHB2, PCDHB17, RAB3IP, SIX1, and WSB1.

15. The method of any one of claims 9-13, wherein the one of more TR activator genes are selected from the group consisting of ADGRV1, AFF3, ALDH5A1, ALX1, AMH, B4GALNT4, C14orf39, CHKB-CPT1B, CPT1B, DOC2GP, DPY19L2, DSG2, FAM157A, FAM157B, FOXD4L4, FSIP2, GDF1, GRIN3B, H2BFXP, L3MBTL1, LIN28B, LINC00649, LINC01021, LINC01116, NAALAD2, PAQR6, members of the alpha clustered protocadherin locus A2-11, members of the beta clustered protocadherin locus B2-17, PCDHGB4, PCDHGB6, PLPPR3, PRR5L, RGPD1, SLCO1A2, TSPAN11, TUBB2B, ZCCHC18, ZNF497, and ZNF853.

16. The method of any one of claims 9-13, wherein the one of more TR activator genes are selected from the group consisting of AC108142.1, AGA, AQP7P1, AQP7P3, BAHD1, BBOX1, C11orf35 (LMNTD2), CASC9, CBX2, CCDC144NL, CHRM3, CPAMD8, FAR2P1, FAR2P2, FAR2P3, FIRRE, IGF2BP1, LINC00649, LINC02315, LOC644919, MED15P9, PCAT7, PKP3, POTEE, POTEF, PURPL, RGPD2, WDR72, WRN, and LMNB1.

17. The method of claim 16, wherein the TR activator gene is PURPL.

18. The method of any one of claims 9-17, wherein the one or more inhibitors of one or more tissue regeneration (TR) activator genes is inhibitory RNA (RNAi).

19. The method of any one of claims 8-18, wherein the one or more iTM factors are administered by viral vector.

20. The method of claim 19, wherein the viral vector is an adeno-associated virus.

21. The method of any one of claims 1-20, wherein the cells are human.

22. The method of any one of claims 1-20, wherein the cells are canine.

23. The method of any one of claims 1-20, wherein the cells are feline.

24. The method of any one of claims 1-23, wherein the one or more endocrine factors and/or one or more iTM factors are administered to the cell for at least 1 day, at least 2 days, at least 3 days, at least 4 days, at least 5 days, at least 6 days, at least 7 days, at least 8 days, at least 9 days, at least 10 days, at least 11 days, at least 12 days, at least 12 days, at least 13 days, at least 14 days, at least 15 days, at least 16 days, at least 17 days, at least 18 days, at least 19 days, or at least 20 days.

25. The method of any one of claims 1-23, wherein the one or more endocrine factors and/or one or more iTM factors are administered to the cell for up to 1 day, up to 2 days, up to 3 days, up to 4 days, up to 5 days, up to 6 days, up to 7 days, up to 8 days, up to 9 days, up to 10 days, up to 11 days, up to 12 days, up to 12 days, up to 13 days, up to 14 days, up to 15 days, up to 16 days, up to 17 days, up to 18 days, up to 19 days, or up to 20 days.

26. The method of any one of claims 1-25, wherein the cell is a pluripotent stem cell.

27. The method of claim 26, wherein the pluripotent stem cell is derived from a somatic cell.

28. The method of claim 27, wherein the somatic cell is selected from the group consisting of cardiac cells, stomach cells, neural cells, lung cells, cells of the ear, cells of the olfactory system, reproductive cells, pancreatic cells, gastrointestinal cells, thyroid cells, epithelial cells, bladder cells, blood cells, respiratory tract cells, salivary gland cells, adipocytes, cells of the eye, liver cells, muscle cells, kidney cells, and immune system cells.

29. The method of any one of claims 8-28, wherein the one or more iTM factors are administered in vitro.

30. The method of any one of claims 8-29, wherein the one of more iTM factors are administered in vivo.

31. The method of any one of claims 1-30, wherein the cell has reduced transcription of the TERT gene compared to the level of transcription of the TERT gene in the cell prior to administration of the one or more endocrine factors and/or the one or more iTM factors.

32. The method of any one of claims 1-31, wherein the cell has reduced transcription of the MANEAL gene compared the level of transcription of the MANEAL gene in the cell prior to administration of the one or more endocrine factors and/or the one or more iTM factors.

33. The method of any one of claims 1-32, wherein the cells has increased COX7A1 expression compared the level of transcription of the COX7A1 gene in the cell prior to administration of the one or more endocrine factors and/or the one or more iTM factors.

34. The method of any one of claims 1-33, wherein the cells has increased COL1A1 expression compared the level of transcription of the COL1A1 gene in the cancer cell prior to administration of the one or more endocrine factors and/or the one or more iTM factors.

35. A method of maturing a cancer cell with an embryonic pattern of gene expression and a glycolytic phenotype, said method comprising administering one or more endocrine factors to said cancer cell.

36. The method of claim 35, wherein the endocrine factors are selected from the group consisting of T3, T4, cortisol, dexamethasone, IGF2, growth hormone, proopiomelanocortin (POMC) and POMC derivative peptides, wherein POMC derivative peptides are N-terminal peptide of proopiomelanocortin (NPP), alpha melanotropin (αMSH), beta-melanotropin (βMSH), delta-melanocyte-stimulating hormone (δMSH), epsilson-melanocyte-stimulating hormone (εMSH), corticotropin, beta-lipotropin, gamma lipotropin, beta-endorphin, and met-enkephalin.

37. The method of claim 36, wherein the endocrine factors are 1) T3 or T4; and 2) one or more of cortisol, dexamethasone, proopiomelanocortin (POMC) and POMC derivative peptides, a MAP kinase activator, FGF7, IGF2, and Growth hormone (GH), wherein the POMC derivatives are N-terminal peptide of proopiomelanocortin (NPP), alpha melanotropin (αMSH), beta-melanotropin (βMSH), delta-melanocyte-stimulating hormone (δMSH), epsilson-melanocyte-stimulating hormone (εMSH), corticotropin, beta-lipotropin, gamma lipotropin, beta-endorphin, and met-enkephalin, wherein the MAP kinase is an ERK1/2 activator, e.g., baicalein or baicalin, and lamin A.

38. The method of claim 36 or claim 37, wherein the endocrine factors are 1) T3 or T4; and 2) one or more of cortisol, dexamethasone, FGF7, IGF2, and Growth hormone (GH).

39. The method of claim 36 or claim 37, wherein the endocrine factors are 1) T3 or T4; and 2) ERK1/2 activator.

40. The method of claim 37 or claim 38, wherein the endocrine factors are 1) T3 or T4; and 2) FGF7.

41. The method of claim 36 or claim 37, wherein the endocrine factors are 1) T3 or T4; and 2) one or more of proopiomelanocortin (POMC) and POMC derivative peptides, wherein the POMC derivatives are N-terminal peptide of proopiomelanocortin (NPP), alpha melanotropin (αMSH), beta-melanotropin (βMSH), delta-melanocyte-stimulating hormone (δMSH), epsilson-melanocyte-stimulating hormone (εMSH), corticotropin, beta-lipotropin, gamma lipotropin, beta-endorphin, and met-enkephalin.

42. The method of any one of claims 35-41, further comprising administering one or more induced tissue maturation (iTM) factors.

43. The method of claim 42, wherein the one or more iTM factors are one or more tissue regeneration (TR) inhibitory genes, one or more inhibitors of one or more tissue regeneration (TR) activator genes, or a combination thereof.

44. The method of claim 43, wherein the one of more TR inhibitory genes are selected from the group consisting of ACAT2, C18orf56, CAT, COMT, COX7A1, DYNLT3, ELOVL6, FDPS, IAH1, INSIG1, LOC205251, MAOA, RPS7, SHMT1, TRIM4, TSPYL5, or ZNF280D.

45. The method of claim 43, wherein the one of more TR inhibitory genes are selected from the group consisting of ADIRF, C10orf11, CAT, CCDC144B, COMT, COX7A1, KRBOX1, LINC00654, LINC00839, LINC01116, MEG3, MIR4458HG, PCDHGA12, PCDHGB3, PCDHGB5, PLPP7 (PPAPDC3), POMC, PRR34, PRSS3, PTCHD3, PTCHD3P1, SPESP1, TRIM4, USP32P1, ZNF300P1, or ZNF572.

46. The method of claim 43, wherein the one of more TR inhibitory genes are selected from the group consisting of ALS2CR11, C2CD6, ANKRD7, ANKRD65, BACE2, BHMT2, C22orf26, CADPS2, CALHM2, CCDC36, CCDC89, CCDC125, CCDC144B, CLDN11, CTSF, DDX43, DNAJC15, EGFLAM, ESPNL, FAM24B, FGF7, FKBP9L, FLG-AS1, FRG1B, GPAT2, GYPE, HENMT1, HIST2H2BA, IRAK4, LINC00865, LOC283788, LOC100233156, LRRK2, MAP10, MEG8, MEG9, MIRLET7HG, NKAPL, PAX8-AS1, PRPH2, PRR34-AS1, RP5-1043L13.1, RP11-134021.1, SVIL-AS1, TEKT4P2, ZNF578, ZNF585B, ZNF736, or ZNF790-AS1.

47. The method of claim 46, wherein the TR inhibitory gene is FGF7.

48. The method of any one of claims 43-47, wherein the one of more TR activator genes are selected from the group consisting of AFF3, CBCAQH03 5, DLX1, DRD1IP, F2RL2, FOXD1, LOC728755, LOC791120, MN1, OXTR, PCDHB2, PCDHB17, RAB3IP, SIX1, and WSB1.

49. The method of any one of claims 43-47, wherein the one of more TR activator genes are selected from the group consisting of ADGRV1, AFF3, ALDH5A1, ALX1, AMH, B4GALNT4, C14orf39, CHKB-CPT1B, CPT1B, DOC2GP, DPY19L2, DSG2, FAM157A, FAM157B, FOXD4L4, FSIP2, GDF1, GRIN3B, H2BFXP, L3MBTL1, LIN28B, LINC00649, LINC01021, LINC01116, NAALAD2, PAQR6, members of the alpha clustered protocadherin locus A2-11, members of the beta clustered protocadherin locus B2-17, PCDHGB4, PCDHGB6, PLPPR3, PRR5L, RGPD1, SLCO1A2, TSPAN11, TUBB2B, ZCCHC18, ZNF497, and ZNF853.

50. The method of any one of claims 43-47, wherein the one of more TR activator genes are selected from the group consisting of AC108142.1, AGA, AQP7P1, AQP7P3, BAHD1, BBOX1, C11orf35 (LMNTD2), CASC9, CBX2, CCDC144NL, CHRM3, CPAMD8, FAR2P1, FAR2P2, FAR2P3, FIRRE, IGF2BP1, LINC00649, LINC02315, LOC644919, MED15P9, PCAT7, PKP3, POTEE, POTEF, PURPL, RGPD2, WDR72, WRN, and LMNB1.

51. The method of claim 50, wherein the TR activator gene is PURPL.

52. The method of any one of claims 43-51, wherein the one or more inhibitors of one or more tissue regeneration (TR) activator genes is inhibitory RNA (RNAi).

53. The method of any one of claims 43-52, wherein the one or more iTM factors are administered by viral vector.

54. The method of claim 53, wherein the viral vector is an adeno-associated virus.

55. The method of any one of claims 35-54, wherein the cancer cell is human.

56. The method of any one of claims 35-55, wherein the cancer cells are canine.

57. The method of any one of claims 35-56, wherein the cells are feline.

58. The method of any one of claims 35-57, wherein the one or more endocrine factors and/or one or more iTM factors are administered to the cell for at least 1 day, at least 2 days, at least 3 days, at least 4 days, at least 5 days, at least 6 days, at least 7 days, at least 8 days, at least 9 days, at least 10 days, at least 11 days, at least 12 days, at least 12 days, at least 13 days, at least 14 days, at least 15 days, at least 16 days, at least 17 days, at least 18 days, at least 19 days, or at least 20 days.

59. The method of any one of claims 35-57, wherein the one or more endocrine factors and/or one or more iTM factors are administered to the cell for up to 1 day, up to 2 days, up to 3 days, up to 4 days, up to 5 days, up to 6 days, up to 7 days, up to 8 days, up to 9 days, up to 10 days, up to 11 days, up to 12 days, up to 12 days, up to 13 days, up to 14 days, up to 15 days, up to 16 days, up to 17 days, up to 18 days, up to 19 days, or up to 20 days.

60. The method of any one of claims 35-59, wherein the cancer cell is a carcinoma cell.

61. The method of any one of claims 35-60, wherein the cancer cell is an adenocarcinoma cell.

62. The method of any one of claims 35-61, wherein the cancer cell is a sarcoma cell.

63. The method of any one of claims 35-62, wherein the cancer cell has reduced transcription of the TERT gene compared to the level of transcription of the TERT gene in the cancer cell prior to administration of the one or more endocrine factors and/or the one or more iTM factors.

64. The method of any one of claims 35-63, wherein the cells has reduced transcription of the MANEAL gene compared the level of transcription of the MANEAL gene in the cancer cell prior to administration of the one or more endocrine factors and/or the one or more iTM factors.

65. The method of any one of claims 35-64, wherein the cells has increased COX7A1 expression compared the level of transcription of the COX7A1 gene in the cell prior to administration of the one or more endocrine factors and/or the one or more iTM factors.

66. The method of any one of claims 35-65, wherein the cells has increased COL1A1 expression compared the level of transcription of the COL1A1 gene in the cancer cell prior to administration of the one or more endocrine factors and/or the one or more iTM factors.
